(19) United States
(12) Patent Application Publication
(10) Pub. No.: US 2024/0207046 A1
(43) Pub. Date: Jun. 27, 2024

(54) PROSTHETIC HEART VALVE

(71) Applicant: EDWARDS LIFESCIENCES CORPORATION, Irvine, CA (US)

(72) Inventors: Nikolai Gurovich, Hadera (IL); Michael Bukin, Pardes Hanna (IL); Anatoly Dvorsky, Haifa (IL); Eran Grosu, Kfar-Saba (IL); Noam Nir, Pardes-Hanna (IL); Joseph Mordechai Leichner, Netanya (IL); Elena Sherman, Pardes Hana (IL); Guillermo W. Moratorio, Laguna Beach, CA (US)

(21) Appl. No.: 18/522,551

(22) Filed: Nov. 29, 2023

Related U.S. Application Data
(63) Continuation of application No. PCT/US2022/032983, filed on Jun. 10, 2022.
(60) Provisional application No. 63/347,384, filed on May 31, 2022.
(60) Provisional application No. 63/298,130, filed on Jan. 10, 2022.
(60) Provisional application No. 63/278,636, filed on Nov. 12, 2021.
(60) Provisional application No. 63/209,904, filed on Jun. 11, 2021.

(51) Int. Cl. A61F 2/24

(52) U.S. Cl. CPC A61F 2/2418; A61F 2220/0008; A61F 2220/0025

(57) ABSTRACT

A prosthetic heart valve comprises a radially expandable frame comprising an outflow end and an inflow end, and a plurality of valve leaflets disposed within and coupled to the frame. Each leaflet comprises a main body having an outflow edge portion and an inflow edge portion, wherein the leaflets are configured to move between an open state and a closed state in which the outflow edge portions coapt with each other and block the flow of blood through the frame from the outlet end to the inlet end, wherein the inflow edge portion of each leaflet includes a movable portion that can move radially inwardly when the leaflets move to the closed state to assist with the coaptation of the outflow edge portions of the leaflets and radially outwardly when the leaflets move to the open state.

### CROSS REFERENCE TO RELATED APPLICATIONS

This application is a continuation of PCT Application No. PCT/US2022/032983, filed Jun. 10, 2022, which claims the benefit of U.S. Provisional Application No. 63/347,384, filed May 31, 2022, U.S. Provisional Application No. 63/298,130, filed Jan. 10, 2022, U.S. Provisional Application No. 63/278,636, filed Nov. 12, 2021, and U.S. Provisional Application No. 63/209,904, filed Jun. 11, 2021. The prior applications are incorporated by reference herein in their entireties.

### FIELD

The present disclosure relates to prosthetic heart valves and delivery assemblies for such prosthetic valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic valves that rely on a mechanical actuator for expansion can be referred to as “mechanically expandable” prosthetic heart valves. The actuator typically takes the form of pull cables, sutures, wires, and/or shafts that are configured to transmit expansion forces from a handle of the delivery apparatus to the prosthetic valve.

The majority of expandable, transcatheter heart valves comprise a cylindrical metal frame or stent and prosthetic leaflets mounted inside the frame. The leaflets may be attached to the frame along their cusp edges (the attachment of the cusp edges can be referred to as a “scallop line”) and at commissure tabs (also referred to as leaflet tabs) of the leaflets. There are certain trade-offs when designing or selecting a leaflet for a prosthetic valve. For example, relatively long leaflets can ensure proper coaptation under the back flow of blood but can result in undesirable pressure gradients across the valve. On the other hand, relatively short leaflets can achieve lower pressure gradients and more desirable hemodynamics, but may affect the ability of the leaflets to fully coapt under the backflow of blood. If the prosthetic valve is intended for use at a single working diameter, the leaflets typically are just long enough to permit proper and full coaptation along the free edges of the leaflets at the intended working diameter. However, if the prosthetic valve is intended for use in a range of working diameters (for example, 26 mm to 29 mm), it is difficult to strike the proper balance between low pressure gradients and full leaflet coaptation. Increasing the length of the leaflets can ensure proper coaptation under a wide range of working diameters but can result in undesirable pressure gradients, while decreasing the length of the leaflets may not permit full coaptation, especially at the upper end of the range of working diameters.

Accordingly, a need exists for improved prosthetic heart valves which desirably reduce pressure gradients across the valve and allow for proper coaptation of the leaflets, especially for prosthetic valves having a range of working diameters.

### SUMMARY

In a representative example, a prosthetic heart valve comprises a radially expandable frame comprising an outflow end and an inflow end; and a plurality of valve leaflets disposed within and coupled to the frame, each leaflet comprising a main body having an outflow edge portion and an inflow edge portion. The leaflets are configured to move between an open state allowing blood to flow through the frame from the inflow end to the outflow end and a closed state in which the outflow edge portions coapt with each other and block the flow of blood through the frame from the outlet end to the inlet end, wherein the inflow edge portion of each leaflet includes a movable portion that can move radially inwardly when the leaflets move to the closed state to assist with the coaptation of the outflow edge portions of the leaflets and radially outwardly when the leaflets move to the open state.

In another representative example, a prosthetic heart valve comprises a radially expandable frame comprising an outflow end portion, an inflow end portion, a central longitudinal axis extending from the inflow end portion to the outflow end portion, a plurality of outflow and inflow apices, and a plurality of cantilevered axial extensions, each axial extension being disposed between a pair of adjacent inflow apices; and a plurality of valve leaflets disposed within and coupled to the frame. Each leaflet comprising a main body having an outflow edge portion and an inflow edge portion extending between a pair of adjacent inflow apices and having a movable portion coupled to a respective axial extension; wherein the movable portions of the leaflet inflow edge portions and the axial extensions are configured to move toward the longitudinal axis when the leaflets close under the back flow of blood and away from the longitudinal axis when the leaflets open under the forward flow of blood.

In another representative example, a prosthetic heart valve delivery assembly comprises a delivery apparatus comprising a handle and a shaft having a proximal end portion coupled to the handle and distal end portion; and an expandable prosthetic heart valve coupled to the distal end portion of the shaft. The prosthetic heart valve comprises a radially expandable frame comprising an outflow end, an inflow end, and a plurality of valve leaflets disposed within and coupled to the frame, each leaflet comprising a main body having an outflow edge portion and an inflow edge portion, wherein the leaflets are configured to move between an open state allowing blood to flow through the frame from the inflow end to the outflow end and a closed state in which the outflow edge portions coapt with each other and block the flow of blood through the frame from the outlet end to the inlet end. The inflow edge portion of each leaflet includes a movable portion that can move radially inwardly when the leaflets move to the closed state to assist with the coaptation of the outflow edge portions of the leaflets and radially outwardly when the leaflets move to the open state.

In another representative example, a prosthetic heart valve comprises a radially expandable frame comprising an inflow end, and outflow end, a circumferentially extending row of cells, a plurality of axially extending first posts having first ends within the cells, a plurality of axially extending second posts having second ends within the cells, wherein each of the first posts is aligned within one of the second posts along a length of the frame to form a pair of first and second posts, and a plurality of actuator members configured to radially expand the frame from a radially compressed state to a radially expanded state. When the frame is in the radially compressed state, the first and second ends are axially spaced from each other and when the frame is in the radially expanded state, the first and second ends contact each other to prevent overexpansion of the frame. A plurality of leaflets disposed inside the frame are configured to regulate the flow of blood in one direction through the frame.

In another representative example, a prosthetic heart valve comprises a radially expandable frame comprising an outflow end portion, an inflow end portion, a plurality of outflow and inflow apices, and a plurality of cantilevered axial extensions, each axial extension being disposed between a pair of adjacent inflow apices; and a plurality of valve leaflets disposed within and coupled to the frame. Each leaflet comprising a main body having an outflow edge portion and an inflow edge portion coupled to a respective axial extension. The leaflet inflow edge portions and the axial extensions are configured to move laterally toward adjacent inflow apices when a force is applied to the axial extensions.

In another representative example, a prosthetic heart valve comprises a radially expandable frame comprising an inflow end, and outflow end, and a plurality of struts arranged to form a circumferentially extending row of struts forming the inflow end, wherein one or more selected struts have at least one opening extending therethrough; and a plurality of leaflets disposed inside the frame and configured to regulate the flow of blood in one direction through the frame. Each leaflet comprising an outflow edge portion and an inflow edge portion. The inflow edge portions of the leaflets are coupled to the selected struts of the frame with sutures extending through the openings.

In another representative example, a prosthetic heart valve comprises a radially expandable frame comprising an outflow end, an inflow end, and a central longitudinal axis extending from the inflow end portion to the outflow end portion; and a plurality of valve leaflets disposed within and coupled to the frame, each leaflet comprising a main body having an outflow edge and an inflow edge, and two commissure tabs on opposite sides of the main body, wherein the leaflets are configured to move between an opened state allowing blood to flow through the frame from the inflow end to the outflow end and a closed state in which the outflow edges coapt with each other and block the flow of blood through the frame from the outlet end to the inlet end; wherein each commissure tab is paired with a commissure tab of an adjacent leaflet to form a plurality of commissures coupled to respective commissure support portions of the frame, wherein the leaflets define an outflow channel that is tapered toward the outflow edges of the leaflets when the leaflets are in the opened state.

In another representative example, a prosthetic heart valve comprises a radially expandable frame comprising an outflow end and an inflow end; and a plurality of valve leaflets disposed within and coupled to the frame, each leaflet comprising a main body having an outflow edge and an inflow edge, and two commissure tabs on opposite sides of the main body, wherein the leaflets are configured to move between an opened state allowing blood to flow through the frame from the inflow end to the outflow end and a closed state in which the outflow edges coapt with each other and block the flow of blood through the frame from the outlet end to the inlet end; wherein each commissure tab is paired with a commissure tab of an adjacent leaflet to form a plurality of commissures coupled to respective commissure support portions of the frame and having inflow ends and outflow ends, wherein the leaflets are tensioned across the outflow edges of the leaflets when the leaflets are in the opened state.

In another representative example, a leaflet for a prosthetic heart valve comprises a main body comprising an inflow edge, an outflow edge, a longitudinal axis, and a pair of opposing commissure tabs, each commissure tab having an inflow end and an outflow end, and extending from a respective side of the main body at an angle greater than zero relative to the longitudinal axis of the main body.

In another representative example, a method for assembling a prosthetic heart valve comprises positioning within a radially expandable frame a leaflet assembly comprising a plurality of leaflets, each leaflet having an inflow edge, an outflow edge, and a pair of opposing commissure tabs, each commissure tab being paired with a commissure tab of an adjacent leaflet to form a plurality of leaflet commissures having inflow ends and outflow ends, wherein the frame comprises a plurality of commissure support portions; stretching each leaflet between its respective commissure tabs and along the outflow edge to position each leaflet commissure adjacent a commissure support portion of the frame; and coupling each commissure to its respective commissure support portion of the frame, wherein the leaflets of the leaflet assembly are configured to move between an opened state allowing blood to flow through the frame from the inflow end to the outflow end and a closed state in which the outflow edges coapt with each other and block the flow of blood through the frame from the outlet end to the inlet end.

In another representative example, a prosthetic heart valve comprises a radially expandable and compressible frame comprising an outflow end portion, an inflow end portion having a plurality of inflow apices, and a plurality of cantilevered axial extensions, each axial extension having a fixed end and free end disposed between a pair of adjacent inflow apices; and a plurality of valve leaflets disposed within and coupled to the frame, each leaflet comprising a main body having an outflow edge portion and an inflow edge portion coupled to a respective axial extension; wherein the leaflet inflow edge portions are secured to the free ends of the axial extensions.

In another representative example, a prosthetic heart valve comprises a radially expandable frame comprising an inflow end, an outflow end, a plurality of axially extending first posts, and a plurality of axially extending second posts, wherein selected pairs of axially aligned first and second posts form a first set of selected posts and other selected pairs of axially aligned first and second posts form a second set of selected posts. The frame further comprises a first set of nuts coupled to the second posts of the first set of selected posts and a second set of nuts coupled to the second posts of the second set of selected posts, wherein the first set of nuts differ in at least one dimension from the second set of nuts; a plurality of first actuator members extending through the first set of selected posts and the first set of nuts, and a plurality of second actuator members extending through the second set of selected posts and the second set of nuts, wherein the first actuator members are configured to rotate in a first direction and the second actuator members are configured to rotate in a second direction, the first and second actuator members being configured to radially expand the frame from a radially compressed state to a radially expanded state; and a plurality of leaflets disposed inside the frame and configured to regulate the flow of blood in one direction through the frame.

In another representative example, a prosthetic heart valve comprising a radially expandable frame comprising an inflow end, an outflow end, and a plurality of axially extending posts, at least one post including an inner bore extending therethrough and an aperture extending from an external surface of the frame to the inner bore of the post; and a plurality of leaflets disposed inside the frame and configured to regulate the flow of blood in one direction through the frame.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of an exemplary example of a prosthetic heart valve and a frame thereof.

FIG. 2 is a perspective view of the prosthetic valve and frame of FIG. 1.

FIG. 3 is a side view the frame of FIGS. 1-2.

FIG. 4 is a plan view of one of the leaflets of the prosthetic valve of FIGS. 1-2 in a flat configuration.

FIGS. 5-6 are top plans view of the prosthetic valve of FIGS. 1-2 shown with the leaflets in an open position and a closed position, respectively.

FIG. 7 is a bottom plan view of a prosthetic heart valve with an outer skirt and/or one or more leaflets extending into the inflow end of the valve.

FIG. 8 is a plan view of an alternative example of a frame for a prosthetic heart valve in a flattened configuration.

FIG. 9 is a perspective view a prosthetic heart valve including the frame of FIG. 8.

FIG. 10 is an enlarged view of a section of the prosthetic valve of FIG. 9 with an outer skirt mounted thereto.

FIG. 11 is a plan view of another example of a frame for a prosthetic heart valve in a flattened configuration.

FIGS. 12-13 are side views of a single set of cells of the frame of FIG. 11 shown in a radially expanded state.

FIGS. 14A and 14B are elevation and cross-sectional views, respectively, of a commissure support member of the frame of FIG. 11 and a commissure formed by two leaflets mounted to the commissure support member.

FIG. 15 is a side view of another example of a frame for a prosthetic valve in a radially expanded state.

FIGS. 16A-16B are enlarged side views of an axial extension of the frame of FIG. 15.

FIGS. 17A-17B are an enlarged side views of an alternative example of an axial extension of the frame of FIG. 15.

FIG. 18 is a side view of the frame of FIG. 15 with an outer skirt mounted thereto.

FIG. 19 is a plan view of another example of a frame for a prosthetic heart valve shown in a flattened configuration.

FIG. 20 is a perspective view of the frame of FIG. 19.

FIG. 21 is a perspective view of a prosthetic heart valve including the frame of FIGS. 19-20.

FIG. 22 is a side view of a single set of cells of another example of a frame for a prosthetic valve in a radially compressed state.

FIG. 23 is an enlarged view of an inflow end of the frame of FIG. 22.

FIG. 24 is an enlarged side view of a single set of cells of an alternative example of a frame for a prosthetic valve in a radially compressed state.

FIG. 25 is an enlarged view of a portion of one of the struts of the single set of cells of the frame of FIG. 24.

FIG. 26 is a perspective view of a prosthetic heart valve including the frame of FIGS. 19-21.

FIG. 27 is a plan view of one of the leaflets of the prosthetic valve of FIG. 26 shown in a flattened configuration.

FIGS. 28A-28C are schematic illustrations of a process of forming commissures of a valvular structure of the prosthetic valve of FIG. 26.

FIG. 29 is a plan view of a leaflet with longitudinally oriented tabs shown in a flattened configuration.

FIG. 30 is a top plan view of a prosthetic heart valve including a plurality of the leaflet of FIG. 29, shown with the leaflets in an open position.

FIG. 31 is top plan view of the prosthetic valve of FIG. 26 shown with the leaflets in an open position.

FIG. 32 is a side elevation view of a delivery apparatus for the prosthetic heart valves of the present disclosure, according to one example.

FIG. 33 is a plan view of another example of a frame for a prosthetic heart valve shown in a flattened configuration.

FIG. 34 is a plan view of another example of a frame for a prosthetic heart valve shown in a flattened configuration.

FIG. 35 is a plan view of a section of another example of a frame for a prosthetic heart valve, shown in a flattened configuration.

FIG. 36 is a side view of an alternative set of cells for the frame of FIGS. 19-21.

FIGS. 37-44 are enlarged side views of axial extensions according to various examples.

FIG. 45A is bottom plan view of a frame having a plurality of axial extensions, according to another example.

FIG. 45B is an enlarged view of one of the axial extensions of FIG. 45A.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” and “connected” generally mean physically, mechanically, chemically, magnetically, and/or electrically coupled or linked and does not excluded the presence of intermediate elements between the coupled or associated items absent specific contrary language. As used herein, “and/or” means “and” or “or”, as well as “and” and “or”.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while the distal motion of the device is motion of the device away from the user and toward the implantation site (for example, into the patient's body). The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such has the longitudinal axis of the prosthetic heart valve).

It should be understood that the disclosed examples can be adapted for delivering and implanting prosthetic heart valves in any of the native annuluses of the heart (for example, the aortic, pulmonary, mitral, and tricuspid annuluses), and can be used with any of the various delivery devices for delivering the prosthetic heart valve using any of a number of delivery approaches (for example, retrograde, antegrade, transseptal, transseptal, transventricular, transatrial, etc.). Although the examples of delivery apparatuses disclosed herein are described in the context of being to implant a prosthetic heart valve, the delivery apparatuses can be used to deliver and implant any of various medical implants within the body, including, but not limited to, venous valves, stents, grafts, heart valve repair devices, etc.

### Examples of the Disclosed Technology

Described herein are examples of prosthetic implants, such as prosthetic heart valves, that can be implanted within any of the native valves of the heart (for example, the aortic, mitral, tricuspid, and pulmonary valves). The present disclosure also provides frames for use with such prosthetic implants. The frames can include struts having differing shapes and/or sizes to minimize the overall compressed or crimped profile of the implant and provide sufficient structural strength and rigidity to areas where needed. The spacing between adjacent apices of such frames can be significantly larger than, and in some instances double that of, conventional frames.

Prosthetic valves including the frames described herein can have a plurality of leaflets mounted within the frame in such a way that a portion of the scallop line of the leaflets extends between the apices of the frame and is free to deflect radially inwardly and outwardly relative to the inside of the frame during the working cycle of the prosthetic valve. In some instances, the frames of the present disclosure can include axial extensions disposed between the apices to which those portions of the scallop line of the leaflets extending between the apices can be coupled. These axial extensions can be configured such that both the axial extension and the portions of the leaflets coupled thereto can deflect radially inwardly and outwardly relative to inside of the frame. Advantageously, shorter designed leaflets can be utilized in the disclosed prosthetic valves due to the radially inwardly motion of the leaflets and/or axial extensions of the frame. The inwardly movement of these features allow for proper coaptation of the shorter leaflets and improve the pressure gradients across the prosthetic valve by reducing the obstruction to blood flow, over conventional prosthetic valves. In addition, the shorter leaflets can decrease the risk of curtaining (blocking) the coronary ostia with the leaflets, such as can occur in valve-in-valve procedures.

In some instances, the frames of the present disclosure can also include axial extensions configured to deflect laterally in a direction toward an apex at one end of the frame. Some or all of these axial extensions can also have the added functionality of the axial extensions described above. For example, the axial extensions which are configured to deflect laterally, can also be configured to deflect inwardly and outwardly relative to the inside of the frame. As with the first type of axial extensions described above, these laterally deflecting axial extensions can also be coupled to the leaflets of the prosthetic valve such that both the leaflets and axial extensions can deflect laterally. Such lateral deflection can, in some cases, permit the axial extensions to move in response to and under the influence of a force applied to the extensions by native tissue. This can, among other things, reduce potential injury to the tissue that may otherwise be caused from contact between the native tissue and the axial extensions during delivery of the prosthetic valve through the vasculature of a patient. Beneficially, a prosthetic valve having a frame which includes such axial extensions can be delivered in a more atraumatic fashion.

The frames included with the prosthetic valves described herein can also have a row of lower struts that form an inflow end of the frame, some of which are selected to have an opening extending therethrough. The opening of these selected struts can be sized and shaped to receive a suture which extends through the opening of the strut and through a connecting skirt attached to the leaflets inside the frame or directly to the leaflets. Axial posts adjacent to these selected struts can also include an indentation which is configured to receive portions of the selected struts which include the openings of the struts. These indentations can be situated such that a portion or the entirety of those sections of the struts forming an opening are received within the indentation as the frames and prosthetic valves are partially or fully compressed, thereby avoiding contact between the selected struts and respective axial posts. In some instances, the portions of the selected struts forming an opening can be formed with a relatively small circumferential width such that contact between the selected struts and axial posts is avoided.

The prosthetic valves described herein can also include a plurality of leaflets that are tensioned along the outflow edges when the leaflets are in an opened state. Each leaflet, for instance, can have tabs which are angled relative to a longitudinal axis of the leaflet such that the leaflet body and the distance between the tabs progressively narrow toward the outflow edge. When mounted to a frame, each leaflet can be stretched along its outflow edge and between its respective tabs in such a way that the narrowest distance between the tabs at the outflow edge is equal to the widest distance between the tabs at its sub-commissure portions. The resulting tension across the leaflets and around the prosthetic valve when the leaflets are opened can define an outflow channel which tapers toward the outflow edges of the valve. This tapered outflow channel can, in some instances, improve the hemodynamics and durability of the valve, such as by avoiding unwanted fluttering at the outflow edges and by offsetting the outflow edges of the leaflets from an inner surface of the frame.

The frames described herein can further comprise actuators (for example, expansion mechanisms) and/or locking mechanisms to enable greater control over the radial compression or expansion of the valve body. Axial posts of the disclosed frames can also be configured to move axially toward and contact one another as the frame is radially expanded to limit or prevent over expansion of such valves. The frames can also comprise a plurality of commissure support members to which leaflet tabs or commissures can be radially or axially inserted and attached.

The prosthetic valves disclosed herein can be radially compressible and expandable between a radially compressed configuration and a radially expanded configuration. Thus, the prosthetic valves can be crimped and retained by an implant delivery apparatus in the radially compressed state during delivery and expanded to the radially expanded state once the prosthetic valve reaches the site of implantation. It is understood that the valves disclosed herein may be used with a variety of implant delivery apparatuses, examples of which are discussed in more detail in the following disclosure.

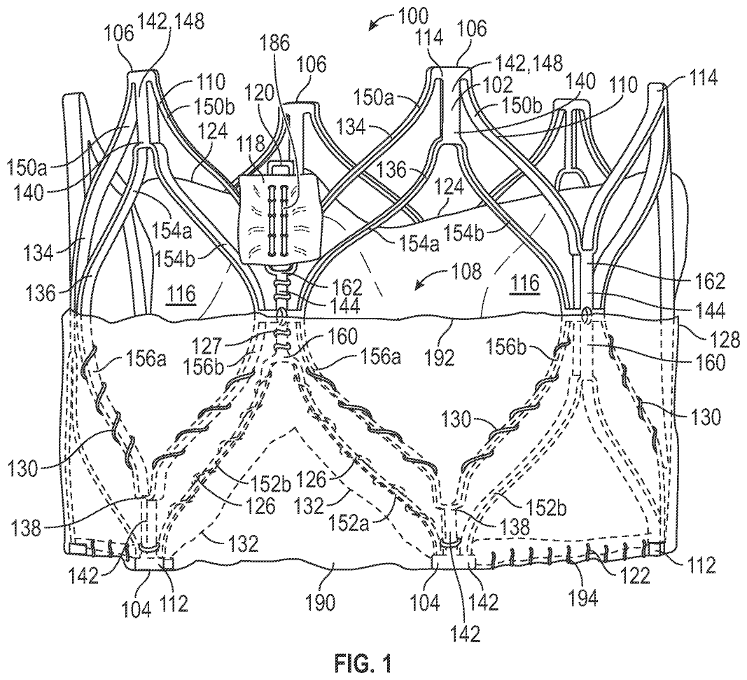

FIG. 1 shows an exemplary prosthetic heart valve 100, according to one example. The prosthetic valve 100 can include an annular stent or frame 102 having an inflow end 104 and an outflow end 106. The prosthetic valve 100 can also include a valvular structure 108 coupled to and supported inside of the frame 102. The valvular structure 108 is configured to regulate the flow of blood through the prosthetic valve 100 from the inflow end 104 to the outflow end 106.

The prosthetic valve 100 can be radially compressible and expandable between a radially compressed configuration and a radially expanded configuration. The frame can include a plurality of circumferentially extending rows of interconnected struts 110 arranged in a lattice-type pattern and forming a plurality of apices 112 at the inflow end 104 of the prosthetic valve 100 and a plurality of similar apices 114 at the outflow end 106 of the prosthetic valve 100.

In the illustrated example, the struts 110 are pivotable or bendable relative to each other to permit radial expansion and contraction of the frame 102. For instance, the frame 102 can be formed (for example, via laser cutting, electroforming or physical vapor deposition) from a single piece of material (for example, a metal tube). As such, the inflow end 104 and the outflow end 106 of the frame 102 can move axially parallel to a longitudinal axis of the prosthetic valve 100 as is radially expanded or compressed, such as during assembly, preparation, or implantation of the prosthetic valve 100.

The frame struts and any components used to construct the frames described herein, can be made of any variety of suitable materials, such as stainless steel, a cobalt chromium alloy, or a nickel titanium alloy (“NitTi”), for example Nitinol. Further details regarding the construction of the frame and the prosthetic valve are described in U.S. Patent Application Nos. 63/085,947, filed Sep. 30, 2020, 63/138,599, filed Jan. 18, 2021, and 63/179,766, filed Apr. 26, 2021, which are incorporated herein by reference.

In other examples, the frame 102 can be constructed of individual components (for example, the struts and fasteners of the frame) and then mechanically assembling and connecting the individual components together. For example, the struts 110 can be pivotably coupled to one another at one or more pivot joints or pivot junctions along the length of each strut. Each of the pivot joints or junctions (for example, hinges) can allow the struts 110 to pivot relative to one another as the frame 102 is radially expanded or compressed. Examples of such frames having pivotably connected struts are disclosed in U.S. Patent Publication Nos. 2018/0153689, 2018/0344456, and 2019/0060057, and WIPO Publication No. 2020/081893, which are incorporated herein by reference.

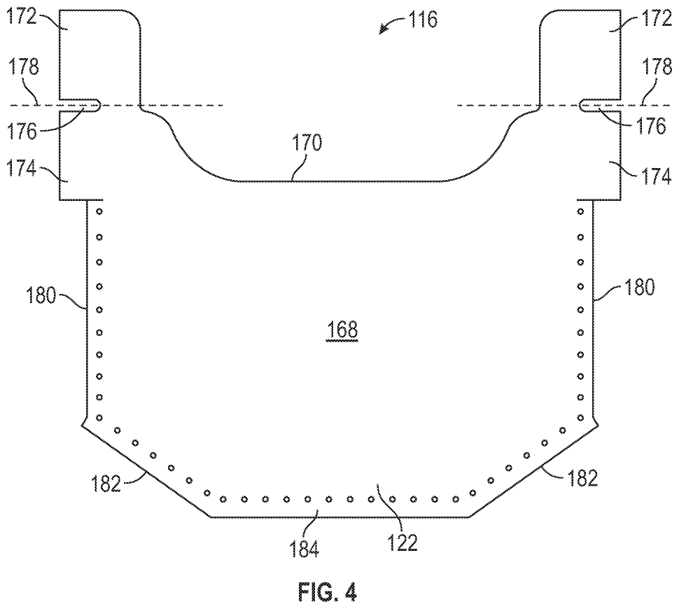

The valvular structure 108 can include a leaflet assembly comprising one or more leaflets 116 (FIG. 4) made of a flexible material. The leaflets 116 can be made from in whole or in part, biological material, biocompatible synthetic materials, or other such materials. Suitable biological material can include, for example, bovine pericardium (or pericardium from other sources). The leaflets 116 can be secured to one another at their adjacent sides to form commissures 118, each of which can be secured to a commissure support member 120 of the frame 102 of valve 100.

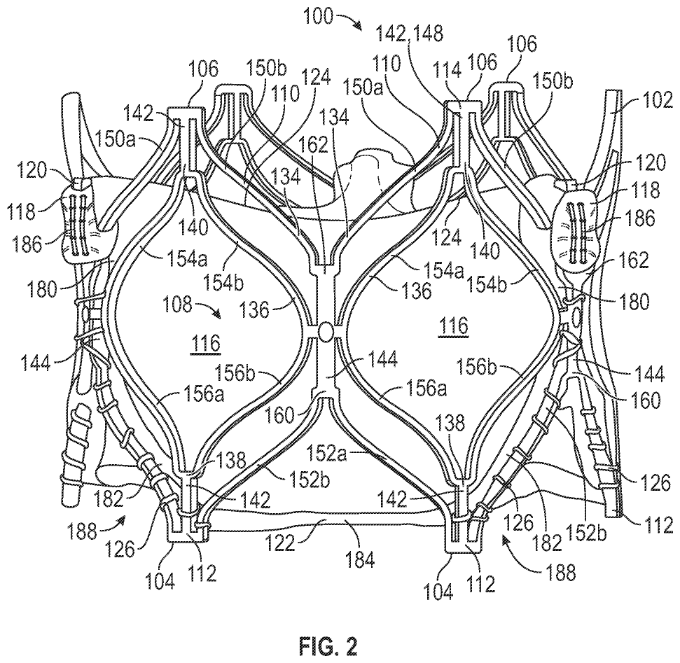

In the example depicted in FIGS. 1 and 2, the valvular structure 108 includes three leaflets 116, which can be arranged to collapse in a tricuspid arrangement. As best shown in FIG. 2, each leaflet 116 can have an inflow edge portion (also referred to as a cusp edge portion) 122 and an outflow edge portion 124. The inflow edge portions 122 of the leaflets 116 can be secured to adjacent struts 152a, 152b of the frame via sutures 126 and define an undulating generally scallop-shaped edge 132 of the valvular structure that follows or tracks portions of the struts 152a, 152b of the frame 102 in a circumferential direction. As such, the inflow edge portions 122 of the leaflets 116 can also be referred to as a “scallop line.”

Further details regarding transcatheter prosthetic heart valves, including the manner in which the valvular structure can be mounted to the frame of the prosthetic valve can be found, for example, in U.S. Pat. Nos. 6,730,118, 7,393,360, 7,510,575, 7,993,394, and 8,252,202, U.S. patent application Ser. No. 15/978,459 (Published as U.S. Publication No. 2018/0325665) and U.S. Provisional Application No. 62/854,702, filed May 30, 2019, all of which are incorporated herein by reference in their entireties.

The prosthetic valve 100 can include one or more skirts or sealing members. In some implementations, the prosthetic valve 100 can include an inner skirt (not shown) mounted on the inner surface of the frame. The inner skirt can function as a sealing member to prevent perivalvular leakage, to anchor the leaflets to the frame, and or to protect the leaflets against damage caused by contact with the frame during crimping and during working cycles of the prosthetic valve.

As shown in FIG. 1 the prosthetic valve 100 can also include an outer skirt 128 mounted on the outer surface of the frame 102 via sutures 130. The outer skirt 128 can function as a sealing member for the prosthetic valve by sealing against the tissue of the native valve annulus and helping to reduce paravalvular leakage past the prosthetic valve. As will be further described herein, the outer skirt 128 can also be sutured to a scallop line of one or more leaflets (for example, inflow edge portions 122) of the valvular structure 108 via sutures 194 such that the outer skirt 128 moves with the leaflets during the working cycles of the prosthetic valve.

The inner and outer skirts can be formed from a variety of suitable biocompatible materials, including any of a variety of synthetic materials, including fabrics (for example, polyethylene terephthalate fabric) or natural tissue (for example, pericardial tissue). Further details regarding the construction and assembly of skirts or sealing members in prosthetic valve can be found, for example, in WIPO Publication No. 2020/198273, which is incorporated herein by reference in its entirety.

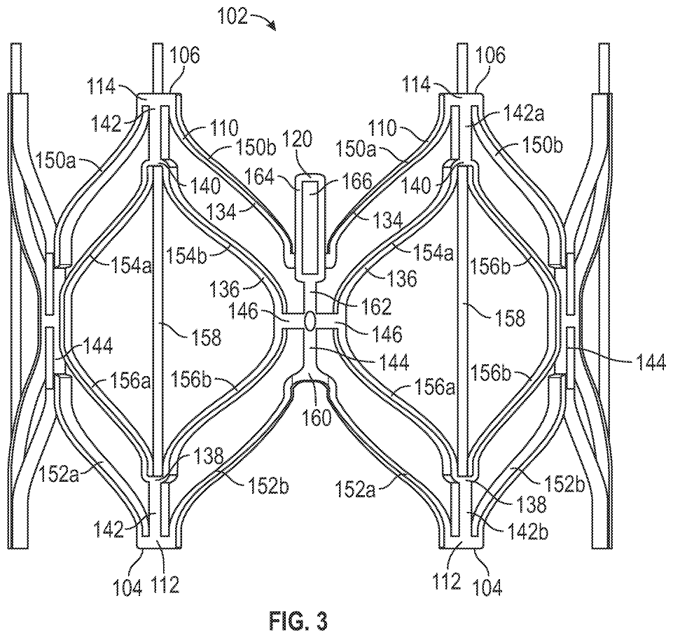

FIGS. 2 and 3 illustrate the prosthetic heart valve 100 and the unitary, lattice frame 102 thereof. For the purpose of illustration and to facilitate discussion of the frame 102, the outer skirt 128 is omitted in FIG. 2, while FIG. 3 illustrates the bare frame 102. While only one side of the frame 102 is depicted in FIG. 3, it should be appreciated that the frame 102 forms an annular structure.

As previously mentioned and as shown in FIGS. 1-3, the frame 102 can comprise a plurality of circumferentially extending rows of interconnected struts 110 arranged in a lattice-type pattern and forming a plurality of first and second apices 112, 114 at the inflow end 104 and at the outflow end 106 of the frame 102, respectively. The struts 110 for instance, define a plurality of first and second cells extending circumferentially around the frame 102. The circumferentially extending cells can include a first cell 134 that is relatively larger than the second cell 136 disposed within the first cell 134.

Each first cell 134 can have an axially extending elliptical shape with first and second apices 112, 114 disposed at the major vertices of the ellipse, at the inflow end 104 and outflow end 106 of the frame 102, respectively. In this way, each first apex 112 can be referred to as an inflow apex and each second apex 114 can be referred to as an outflow apex. Moreover, each second cell 136 can have a circumferentially extending elliptical shape with first and second apices 138, 140 (for example, inflow apex 138 and outflow apex 140) disposed at the minor vertices of the ellipse. As best illustrated in FIGS. 2 and 3, each second cell 136 can be disposed within the outer perimeter of a respective first cell 134.

Although the frame 102 is described herein as having elliptical-shaped first and second cells 134, 136, in other examples, the first and second cells can be configured in a variety of shapes, such as hexagonal, diamond, triangular, tear-drop shaped, rectangular, square, oval, square-oval, etc. For instance, each first cell can be a relatively, larger hexagonal cell having a relatively, smaller diamond-shaped second cell disposed within.

FIGS. 1-3 further show the frame 102 also includes a plurality of axially extending struts, or posts, 142, each of which extends between an apex 138, 140 of a second cell 136 and an apex 112, 114 of a first cell. The illustrated frame 102 also includes a plurality of axially extending struts, or posts, 144, each of which can be disposed between circumferentially adjacent first cells 134.

Each first cell 134 is formed by two upper struts 150a, 150b and two lower struts 152a, 152b. Each upper and lower strut 150, 152 is coupled on one end to a post 142, and on the other end to a post 144. The upper struts 150a, 150b can be part of an upper row of struts that defines the outflow end 106 of the frame 102, and the lower struts 152a, 152b can be part of a lower row of struts that defines the inflow end 104 of the frame 102. Each second cell 136 is formed by two upper struts 154a, 154b and two lower struts 156a, 156b. The lower ends of the upper struts 154a, 154b and the upper ends of the lower struts 156a, 156b, can be connected to lateral extensions 146 of posts 144. The upper ends of the upper struts 154a, 154b, and the lower ends of the lower struts 156a, 156b can be connected to respective posts 142.

As illustrated in FIGS. 1-3, the frame 102 comprises six first cells 134 extending circumferentially in a row, with a second cell 136 disposed within each first cell, and six support posts 144. In other examples, the frame 102 can include a greater or fewer number of first cells 134 within a row, and corresponding greater or fewer number of second cells 136 and/or 144.

As mentioned, the frame 102 can include a plurality of axially extending posts 142 arranged in pairs of posts within each first cell 134. Each pair of posts includes an upper post 142a and a lower post 142b. As best illustrated in FIG. 3, each pair of posts 142a, 142b can be axially aligned with one another. Each post 142a, 142b can include an inner bore (not shown) extending along the length of the post through which an actuator member (for example, a rod) 158 can extend. Each bore extending through its respective post 142a, 142b can, for instance, be configured to engage the rod 158 such that manipulation of the rod 158 causes the first post 142a to move axially relative to the second post 142b. In this example, for instance, rotation of the actuator member 158 in a first direction (for example, clockwise) causes corresponding axial movement of the first and second posts 142a, 142b toward one another, radially expanding the frame 102. In a like manner, rotation of the rod 158 in a second direction (for example, counterclockwise) causes corresponding axial movement of the first and second posts 142a, 142b away from one another to radially compress the frame 102.

In some examples, the bore of at least one post of a pair of posts 142a, 142b is threaded to engage corresponding threads of the actuator member 158 such that rotation of the actuator member 158 causes the first post 142a to move axially relative the second post 142b. For instance, the first post 142a can have internal threads that engage threads of the actuator member 158 and the second post 142b can be non-threaded. Alternatively, the second post 142b can have internal threads that engage threads of the actuator member 158 and the first post 142a can be non-threaded. In another example, both the first post 142a and the second 142b can have threads that engage corresponding threads of the actuator member 158, with the threads of the first post 142a and the corresponding threads of the actuator 158 being oppositely threaded from the threads of the second post 142b and the corresponding threads of the actuator member 158.

In alternative examples, the actuator members 158 can be push-pull members that are configured to radially expand and compress the frame via pulling and pushing, respectively, the actuator members 158. For example, the distal end portions of the actuators members 158 can be fixed axially relative to the second posts 142b and the proximal end portions of the actuator members 158 can be slidably coupled to the first posts 142a, such as by extending through the bores of the first posts 142a. In this manner, proximal movement of the actuator members 158 causes the second posts 142b to move toward the first posts 142a to radially expand the frame, and distal movement of the actuator members 158 causes the second posts 142b to move away from the first posts 142a to radially compress the frame.

Although in the illustrated example each pair of support posts 142a, 142b includes a respective actuator 158, in other examples, one or more pairs of support posts 142a, 142b can be without actuator members 158. Further details regarding the use and construction of the actuator members and corresponding components of a prosthetic valve can be found, for example, in U.S. Patent Application Nos. 63/085,947, 63/138,599, and 63/179,766, which are incorporated herein by reference. Although the frames described herein are radially expanded and/or compressed via a combination of actuators and rods, it should be understood that each of the frames disclosed can be radially expanded and/or compressed a variety of other means, such as pull rods, pull wires, and/or tethers (for example, cables or sutures).

As shown in FIGS. 1-3, the support posts 144 can extend longitudinally and can have an inflow end portion 160 and an outflow end portion 162. The outflow end portion 162 of one or more support posts 144 can include the commissure support member 120 such that a commissure support member 120 is disposed between two outflow apices 114. In particular, the number of commissure support members 120 is equal to the number of leaflets 116. In the illustrated example, there are three leaflets 116, three commissure support members 120 extending from respective support posts 144, and three support posts 144 without commissure support members.

As best illustrated in FIG. 3, each commissure support member 120 can comprise a window 164 formed by the support post 144 that fully encloses or frames an opening 166 extending radially through the thickness of the support post 144. The window 164 of the commissure support member 120 can be configured, for example, to receive a portion of the valvular structure 108, such as the leaflet commissures 118 in order to couple the valvular structure 108 to the frame 102. For instance, the leaflet commissures 118 can be inserted radially though the opening 166 of the window 164 and coupled to the commissure support member 120. In the illustrated example, the window 164 and opening 166 are rectangular in shape; however, in other examples, the window and/or opening of the commissure support can be configured in a variety of shapes, including a square, square-oval, triangular, elliptical, L-shaped, T-shaped, C-shaped, etc.). In other examples, each commissure support member can include an opening at the outflow end portion of the window such that the opening is not fully enclosed and the leaflet commissures can be slid axially within the opening of the commissure support (for example, FIG. 8).

As previously stated, FIG. 3 depicts only one side of the frame 102. Though only one support post 144 comprising a commissure support member 120 is shown in FIG. 3, it should be noted that the frame 102 can comprise any number of support posts 144, any number of which can be configured as commissure support members 120. For instance, as shown in FIGS. 1-3, the frame 102 can comprise six support posts 144, three of which are configured to include commissure support members 120. In some examples, the frame can comprise one, two, or four or more commissure support members.

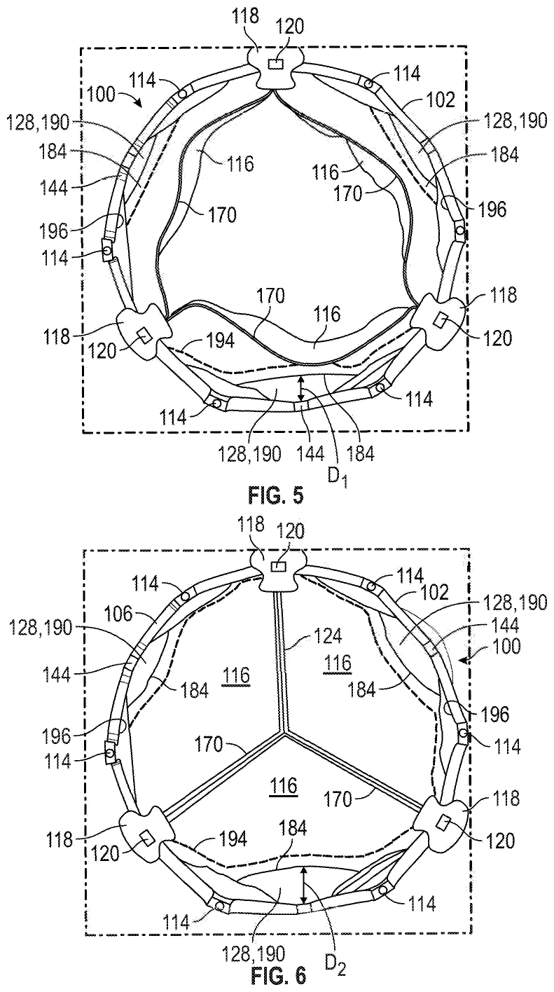

As shown in FIG. 2, the valvular structure 108, and the leaflets 116 thereof, are mounted within of the frame 102. The leaflets 116 of the valvular structure 108 can be coupled, for instance, to one or more commissure support members 120 and/or struts 152a, 152 of the frame. The inflow edge portions 122 of each leaflet 116 can be coupled to the struts 152a, 152b such that the inflow edge portions 122 extend unanchored between pairs of adjacent inflow apices 112. In this way, the leaflets 116 can be mounted within the frame 102 and considered to have a “free” inflow edge portion 122. As further described herein, these portions of the inflow edge portions 122 are movable portions of the leaflet inflow edge portions that are configured to deflect or move radially inwardly and outwardly relative to the frame 102 during the working cycle of the prosthetic valve (FIGS. 5 and 6) to promote coaptation of the outflow edges of the leaflets.

Referring to FIG. 4, each leaflet 116 of the valvular structure 108 can include a main body 168, an outflow edge portion 170, and an inflow edge portion 122, which forms a generally scallop shape. As illustrated in FIG. 4, the outflow edge portion 170 extends between opposing upper and lower tabs 172, 174 disposed on opposite sides of a main body 168 of the leaflet 116. Below each upper tab 172 there is a notch 176 separating the upper tab from a corresponding lower tab 174. An imaginary fold line 178 extends through the notch between each pair of upper and lower tabs. Each upper tab 172 can be folded over and positioned against the lower tab 174 such that the tabs on each side of the main body 168 can form a structurally reinforced commissure tab assembly. Each leaflet 116 of the valvular structure 108 can be secured to one another at their adjacent tabs 172, 174 (for example, the folded and reinforced tabs) to form respective leaflet commissures 118, each of which can be secured to a respective commissure support member 120.

As illustrated in FIG. 4, the inflow edge portion 122 of each leaflet 116, also referred to as the scallop line of the leaflet, comprises angled side edge portions 182, and an apex edge portion 184 extending between the side edge portions 182. Each of the apex edge portions 184 and the side edge portions 182 can be straight or substantially straight such that the inflow edge portion 122 has a truncated V-shape. In other examples, the inflow edge portion 122 can be curved, such as a U-shaped or a parabolic curve. Each leaflet 116 further includes sub-commissure portions comprising axially extending side edge portions 180, each of which extends from a lower tab 174 to an angled side edge portion 182.

As previously mentioned, the leaflets 116 of the valvular structure 108 can be coupled to one or more struts of the frame 102 and/or other soft components of the prosthetic valve 100. For instance and as best illustrated in FIG. 2, the valvular structure 108 can comprise leaflets 116 as described herein, coupled to the commissure support members 120, and one or more of the lower struts 152a, 152b that form the inflow apices 112 of the frame 102. The leaflets 116 of the valvular structure 108 can also be coupled to an outer skirt 128 mounted to the outer surface of the frame 102 (FIGS. 1 and 5-6).

To form a commissure 118, each upper tab 172 of each leaflet 116 can be folded against a respective lower tab 174. Each pair of tabs 172, 174 is then paired with a pair of tabs 172, 174 of an adjacent leaflet to form a commissure 118. As shown in FIG. 2, the commissures 118 formed by the upper and lower tabs 172, 174 of adjacent leaflets 116 can be received radially through the windows 164 (FIG. 3) of the commissure support members 120. As illustrated in FIG. 2, the commissures 118 of the valvular structure 108 can be secured to the commissure support member 120 via sutures 186. In some examples, the valvular structure or leaflet assembly 108 can be preassembled and then mounted to the frame 102. For instance, the valvular structure 108 can be preassembled by connecting (for example, with sutures) each pair of tabs 172, 174 with an adjacent pair of tabs 172, 174 so that all of the leaflets 116 are connected to each other at the commissures 118. The preassembled leaflet assembly can then be positioned inside of the frame 102 and the commissures 118 can be inserted radially through the windows 164 of the commissure support members 120 and secured in place with sutures 186. The remaining portions of the leaflets 116 can be coupled to the frame and/or the skirt 128 of the prosthetic valve, as further described below.

As illustrated in FIG. 2, certain portions of the leaflets 116 can be directly coupled to one or more support posts 144 and/or struts of the frame 102 via sutures 126, 127. For instance, each axially extending side edge portion 180 of each leaflet 116 can be paired with an adjacent side edge portion 180 of an adjacent leaflet 116 and then secured to an adjacent support post 144 below the commissure support member 120 with sutures 127. Sutures 127 can form, for example, whip stitches that extend through the pair of side edges portions 180 and around the support post 144.

Similarly, the angled edge portions 182 of the leaflets 116 can be coupled to one or more lower struts 152 forming the first cells 134. As shown in FIG. 2, the angled edge portions 182 largely track and/or align with the lower struts 152 extending between the inflow apices 112 and the support posts 144. Sutures 126 can be used to connect each edge portion 182 to an adjacent strut 152a or 152b. Sutures 126 can form, for example, whip stitches that extend through each edge portion 182 and around an adjacent strut 152a, 152b. As depicted in FIG. 2, the ends of the angled edge portions 182 nearest the apex edge portions 184 can be coupled to the lower ends of the lower struts 152 and/or the posts 142 that extend between the major and minor vertices 112, 138 of the respective first and second cells 134, 136. In having the angled edge portions 182 coupled to the lower struts 152 and/or post 142 in this way, the apex edge portions 184 are positioned at or substantially at the inflow end 104 and inflow apices 112 of the frame 102. In some examples, the angled edge portions 182 of the leaflets can have a similar length to the lower struts 152 in which they are coupled. In such examples, the axial edge portions 180 can largely track and have a similar length to the support posts 144 coupled thereto.

As best shown in FIG. 2, the apex edge portions 184 of the leaflets 116 extend between adjacent inflow apices 112 unsecured or unanchored to the frame 102 at the inflow end 104. In other words, there is no direct coupling between the frame 102 and the portion of the apex edge portion 184 extending between respective inflow apices 112. Particularly, the apex edge portions 184 extend freely between a post 142 and/or lower strut 152 of one inflow apex 112, and the post 142 and/or lower strut 152 of a corresponding, adjacent inflow apex. In this instance, the apex edge portions 184 span the radial space or gap between the adjacent inflow apices 112.

As mentioned previously, the leaflets 116 forming the valvular structure 108 can be made of a flexible material. In leaving the apex edge portions 184 unanchored to the frame 102, the flexible material of the apex edge portions 184 is free to move radially inwardly and outwardly relative to an inner surface of the frame 102. In particular, the apex edge portions 184 of the leaflets 116 are configured to deflect radially inwardly toward a longitudinal axis of the frame 102 and deflect radially outwardly away from the longitudinal axis and toward an outer boundary of frame 102, for instance, during the working cycle of the prosthetic valve, as will be further described. As used herein, the outer boundary of the frame 102 is the circumference of the outer surface of the frame 102.

Although the apex edge portions 184 of the leaflets 116 are described as having no direct coupling to the frame 102, in some examples, the outer ends of the apex edge portions 184 (those ends near the angled edge portions 182), can be coupled to the frame such that a substantial portion of the apex edge portion 184 is still unanchored to the frame 102 and configured to move radially inwardly toward and outwardly away from the longitudinal axis of the frame 102.

As illustrated in FIG. 2, the apex edge portions 184 of the leaflets 116 can extend between a pair of adjacent inflow apices 112 such that a leaflet 116 extends between every other circumferential gap 188 formed by the inflow apices 112 circumferentially around the frame 102. By way of example, the prosthetic valve 100 depicted in FIG. 2 includes the three leaflets 116 and six inflow apices 112. Each two adjacent inflow apices 112 form a circumferential gap 188 extending therebetween for a total of six circumferential gaps. In this example, each leaflet 116 can be coupled to a respective pair of inflow apices 112 such that a leaflet 116 extends between every other circumferential gap circumferentially around the frame 102 (for example, three of the six circumferential gaps). As such, a circumferential gap 188 without a leaflet extending therebetween, can be said to extend between pairs of inflow apices 112 and in between the apex edge portions 184 of the leaflets 116.

In the illustrated example of FIG. 2, the frame 102 includes six inflow and outflow apices 112, 114, six circumferential gaps, and three leaflets 116 arranged in a tricuspid arrangement. In some examples, however, the frame 102 can have a greater or fewer number of inflow and outflow apices, and corresponding circumferential gaps therebetween, such that the leaflets can be arranged in a different pattern than as shown in FIG. 2.

Turning again to FIG. 1, the illustrated example shows that each apex edge portion 184 of the leaflets 116 can be coupled to one or more soft components of the prosthetic valve 100, including to the outer skirt 128 mounted to the outer surface of the frame 102. The outer skirt 128 can, for instance, comprise an inflow end portion 190 positioned at inflow end 104 of the frame 102 and an outflow end portion 192 positioned between the inflow end 104 and outflow end 106 of the frame 102. As shown in FIG. 1, the outer skirt 128 can extend circumferentially around the outer surface of the frame 102 and extend axially from the inflow end portion 190 to the outflow end portion 192. As best illustrated in FIG. 1, the outer skirt 128 can be coupled to one or more interconnected struts 110 of the frame 102 via sutures 130, such as the lower struts 156 that form the second cells 136. Yet, the outer skirt 128 can be coupled to any other strut of the frame 102.

The inflow edge portion 190 of the outer skirt 128, however, can be left unsecured or unanchored to the frame 102 between one or more pairs of adjacent inflow apices 112. Each pair of adjacent inflow apices 112 of which the outer skirt is unanchored can, for instance, correspond with those inflow apices 112 having a leaflet 116 extending therebetween. In this way, the inflow end portion 190 of the outer skirt 128 is free to be coupled to the apex edge portions 184 of each of the leaflets 116 mounted within the frame 102, for example, via sutures 194. In this example, the outer skirt 128 is configured to prevent leakage between the leaflets 116 and the outer skirt 128 during the working cycle of the prosthetic valve 100 without hindering the radially inwardly and outwardly movement of the apex edge portions 184. In particular, the inflow end portion 190 of the outer skirt 128 can be configured to move radially inwardly and outwardly with the apex edge portion 184 of the leaflets 116.

Referring to FIGS. 5 and 6, the illustrated examples show the outflow end 114 of the prosthetic valve 100 from a “downward” view along the longitudinal axis extending through the center of the frame 102 (for example, from the outflow end to the inflow end of valve 100). FIGS. 5 and 6, illustrate the movement of the valvular structure 108 and outer skirt 128 of the prosthetic valve 100 while in operation, such as when a pressure gradient across the valve forces the leaflets 116 open (for example, when blood flows from the inflow end 112 to the outflow end 114) (FIG. 5) and then causes coaptation of the leaflets under backward pressure (FIG. 6).

As shown in FIGS. 5 and 6, during operation of the valve 100, those portions of the inflow end portion 190 of the outer skirt 128 coupled to the leaflets are configured to move with the apex edge portions 184 as they move radially inwardly and outwardly relative to the inner surface of the frame 102. For instance, while the leaflets 116 are open, the apex edge portions 184 and the portion of the outer skirt 128 coupled thereto can be radially spaced from the inner surface 196 of the frame 102 at a first distance D1. Similarly, the apex edge portions 184 and the outer skirt 128 can be radially spaced from the inner surface 196 at a second distance D2 while the leaflets are closed.

In particular and by way of example, the apex edge portion 184 located in the lower half of FIG. 5 can be radially spaced a first distance D1 from the inner surface of the frame as the pressure gradient causes the leaflets to open and the apex edge portions 184 to move radially outwardly toward the inner surface 196 of the frame. In a like manner, the same apex edge portion 184 shown in the bottom half of FIG. 6 can move radially inwardly toward the longitudinal axis of the frame 102 as the back pressure causes coaptation of the leaflets and draws the free portions of the leaflets 116 (for example, main body 168 and apex edge portion 184) away from the frame 102. The apex edge portion 184 in this case becomes radially spaced from the inner surface 196 at a greater distance D2 when the leaflets 116 are closed, than the distance D1 the apex edge portion 184 is radially spaced when the leaflets 116 are open.

This radial inward movement of the apex edge portions 184 of the leaflets 116 can, for example, promote proper coaptation of the free edges 170 by allowing the main bodies 168 and the free edges 170 of the leaflets to move closer toward the longitudinal axis of prosthetic valve. Thus, it is possible to use relatively shorter leaflets that can reduce the pressure gradient across the prosthetic valve compared with leaflets that are fixed to the frame along their inflow edges. As such, shortened leaflets can be used in combination with the frames described herein to construct prosthetic valves reduce the pressure gradient across the valve, while being able to achieve proper coaptation of the leaflets.

Moreover, this technique of mounting the leaflets to the frame so as to allow movement of the inflow edge portions during valve cycling can ensure full coaptation across a range of working diameters without inducing excessive pressure gradients across the prosthetic valve. The overall size of the leaflets can be selected based on the lower end of the range of working diameters. For example, for a prosthetic valve 100 configured to be radially expanded to a working diameter from 26 mm to 29 mm, the size of the leaflets can be selected to achieve a desired pressure gradient and full coaptation when the frame is expanded to 26 mm. Due to the ability of the inflow edge portions to move inwardly during valve closure, the free edges of the leaflets can still fully coapt when the frame is expanded to a diameter greater than 26 mm. In contrast to known prosthetic valves having a range of working diameters, the leaflets need not be oversized to ensure full coaptation in the upper range of working diameters.

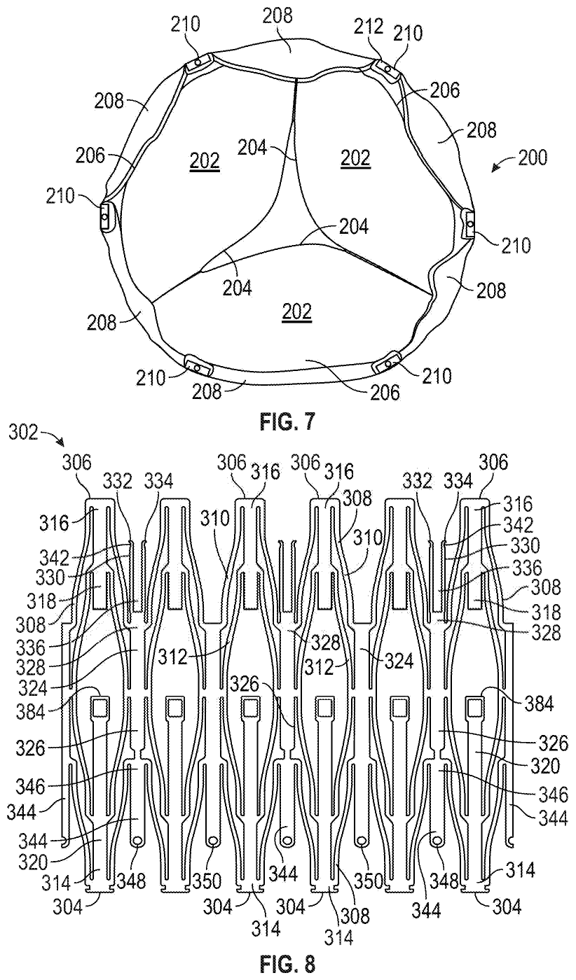

Now referring to FIG. 7, the illustrated example shows an inflow end of a prosthetic valve 200 viewed “upward” along a longitudinal axis extending through the center of the frame 212 (for example, from the inflow end to the outflow end of valve). The prosthetic valve 200 has a valvular structure including three leaflets 202 in a tricuspid arrangement, each leaflet comprising an outflow edge portion 204 and an inflow edge portion 206 coupled to an outer skirt 208. As shown in FIG. 7, in some instances, radial spacing between adjacent pairs of inflow apices 210 of a frame is too great such that an undesirable amount of slack or loose sections in the leaflets 202 and/or outer skirt 208 extend into the inflow end of the valve, which may result in an obstruction of blood flow and increase pressure gradient.

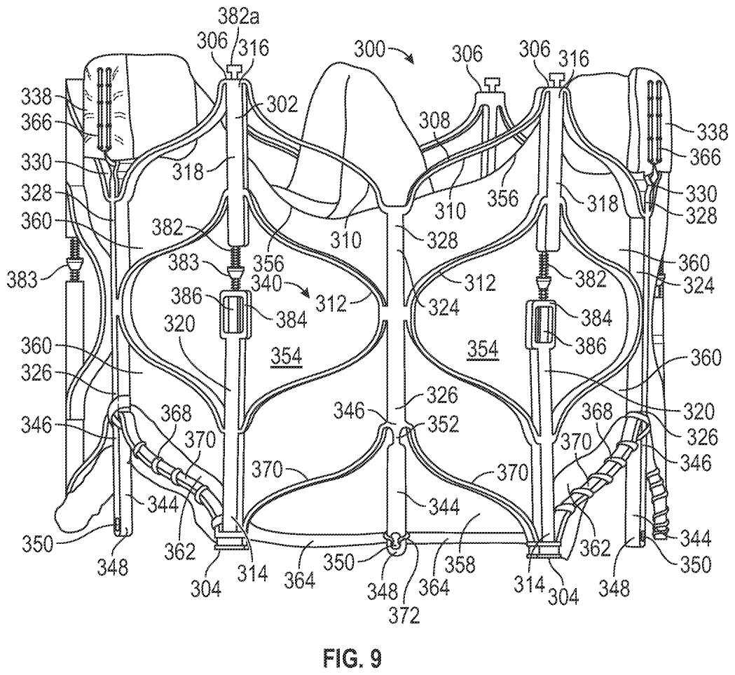

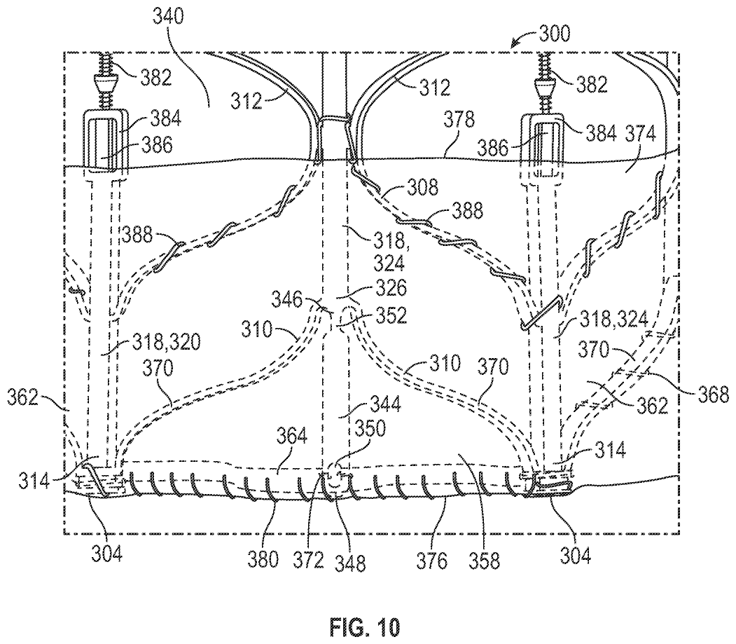

FIG. 8 illustrates an example of a frame 302 for a prosthetic heart valve 300 (FIGS. 9 and 10) in a flattened or unrolled state. The frame 302 can be structurally similar to and function in a similar manner as the described frame 102. For instance, the frame 302 can include an inflow end 304, an outflow end 306, and a plurality of circumferentially extending rows of interconnected struts 308 forming a plurality of first and second elliptical-shaped cells 310, 312 and a plurality of inflow and outflow apices 314, 316 at the inflow end 304 and outflow end 306, respectively. The frame 302 in this example can also include a plurality of axially extending posts 318, 320, and support posts 324, one or more of which having a commissure support member 330. The frame 302 can also include further features extending from one or more of the support posts that are configured to move radially inwardly with one or more leaflets of a valvular structure mounted within the frame 302. As will be further described, the frame 302 can include one or more axial extensions which are configured to deflect inwardly with the leaflets during the working cycle of the valve while also preventing excessive slack or “loose” sections in the scallop line of the leaflets and/or the outer skirt from obstructing blood flow.

As mentioned and as shown in FIG. 8, the frame 302 can include a plurality of axially-extending struts or posts 318, 320. In particular, the frame 302 can include a plurality of first posts 318 extending from and coupled to the outflow apices 316 at the outflow end 306, and a plurality of second posts 320 extending from and coupled to the inflow apices 314 at the inflow end 304. Each first post 318 can be axially aligned with a corresponding second post 320 to define a pair of first and second posts. The posts 318, 320 can function in the same manner as posts 142 (FIGS. 1-3) such that the posts 318, 320 in conjunction with actuator members (described below) are configured to radially expand and/or compress the frame 302.

Each support post 324 can extend longitudinally and have an inflow end portion 326 and an outflow end portion 328. As illustrated in FIG. 8, the outflow end portion 328 of one or more support posts 324 can include a commissure support member 330. The commissure support member 330 can comprise first and second commissure arms 332, 334 defining a commissure opening 336 between them. The commissure opening 336 can extend radially through a thickness of the post 324 and be configured to receive a leaflet commissure 338 of the valvular structure 340 (FIG. 9). In the illustrated example, the commissure opening 336 has a substantially rectangular shape and opens toward the outflow end 306 of the frame 302. In this instance, a commissure formed of a pair of adjacent leaflets can slide axially between the first and second commissure arms 332, 334 and into the opening 336 to mount and support the valvular structure within the frame 302, such as when constructing the prosthetic valve.

In other examples, the commissure opening can have a variety of shapes, such as square, oval, square-oval, triangular, L-shaped, T-shaped, C-shaped, etc. In some examples, the opening 336 can be fully enclosed by the post 324 (for example, the commissure support member 120 of FIG. 3) such that a valvular structure can be slid radially, rather than axially, into the commissure opening.

As shown in FIG. 8, the first and second commissure arms 332, 334 can each include a respective notch or indentation 342. Each indentation 342 can be located along each arm 332, 334 proximate the outflow end 306 of the frame 302. By locating the indentations 342 at the end of each respective arm 332, 334 and near the outflow end 306 of the frame 302, each indentation can be located at or above an upper edge of a leaflet commissure inserted into the opening 336 of the commissure support member 330 (for example, the edge of a leaflet commissure nearest the outflow edge of the leaflets). Each indentation 342 can be configured to receive one or more fasteners, such as sutures, that extend from the indentation 342 of the first commissure arm 332 to the indentation 342 of the second commissure arm 334 (and vice versa). The combination of indentations 342 and one or more fasteners can form a boundary at or near the indentations 342 to prevent or limit axial movement of the leaflet commissure toward the outflow end 306 of the frame 302 and between the first and second commissure arms 332, 334. One or more fasteners and the indentations 342 can also serve to draw respective commissure arms 332, 334 closer to one another to secure the leaflet commissure. For instance, fasteners taut across the distance between the arms 332, 334 can cause the first and second commissure arms 332, 334 to apply a lateral force to the sides of the leaflet commissure. This lateral force applied to the leaflets by the arms 332, 334 can act to secure the leaflet commissure within the opening 336 and limit any radial or axial movement. As such, the commissure support member 330 can also be referred to as a “clasp.” In some examples, each leaflet commissure can also be sutured or otherwise fastened to one another and/or to the arms 332, 334.

As shown in FIG. 8, the inflow end portion 326 of each support post 324 can include a cantilevered strut or axial extension 344 that extends toward the inflow end 304 of the frame 302. In the illustrated example, each axial extension 344 can include a fixed end 346 coupled to a respective support post and a free end 348 that extends toward the inflow end 304 of the frame 302. The axial extensions 344 can have a length such that upon radial expansion of the frame 302, the free ends 348 align with or are located proximate the inflow end of the frame 302 (FIGS. 9 and 10). The free end 348 of one or more axial extensions 344 can also include an aperture 350 extending radially through the thickness of the extension. The aperture 350 can be sized and shaped, for instance, to receive one or more fasteners (for example, sutures) and/or soft components of the prosthetic valve (for example, an outer skirt). In some examples, the axial extensions 344 can include two or more apertures located at various points along its length.

Each axial extension 344 can be formed of a variety of suitable materials, such as stainless steel, a cobalt chromium alloy, or a nickel titanium alloy (“NitTi”), for example Nitinol. In particular examples, each axial extension 344 can be formed of a material with shape memory properties, such as Nitinol. Being made of such materials can, for instance, allow the axial extensions 344 to be configured to move between a straight configuration and a bent or curved configuration. For example, each axial extension 344 in this case can be configured to curve radially along its length between its fixed end 346 and its free end 348 such that the free end 348 extends radially inwardly into the frame 302 (for example, toward a longitudinal axis of the frame 302). In this way and as will be described in further detail, the axial extensions 344 can be coupled to the inflow edge of corresponding leaflets (for example, leaflets 354) in such a way as to allow both the inflow edge of the leaflet and axial extension 344 to move radially inwardly into the frame 302. Such radially inwardly movement can be beneficial, for example, to provide proper leaflet coaptation during the working cycle of a prosthetic valve having the frame 302. By being coupled to leaflets and/or outer skirt, the axial extensions 344 can also prevent undesirable slack or “loose” sections of the leaflets and outer skirt from extending in the way of and obstructing blood flow.

In some examples, the fixed end 346 of the axial extensions 344 can include a narrowed neck portion 352 (FIGS. 9 and 10) by which each axial extension 344 has increased flexibility to bend or curve radially inwardly toward the center of the frame 302. In further examples, when the frame 302 is in a radially expanded configuration, the free end 348 of the axial extensions 344 can extend axially beyond the inflow end 304 of the frame 302, or alternatively, be located between the inflow end 304 and outflow end 306 of the frame 302. Although the illustrated example of FIG. 8 shows the frame 302 comprising six axial extensions 344, in other examples, the frame can comprise any number of greater or fewer axial extensions.

As shown in the illustrated example of FIG. 8, the frame 302 comprises six first cells 310 that can extend circumferentially in a row, with a second cell 312 within each first cell. The frame can also include six pairs of posts 318, 320 coupled to respective pair of cells 310, 312, six support posts 324, six axial extensions 344, and three commissure support members 330. In other examples, however, the frame 302 can comprise a greater or fewer number of each of these components.

FIGS. 9 and 10 illustrate an example of a prosthetic valve 300 in a radially expanded configuration comprising the frame 302. As mentioned, the frame 302 can include a plurality of commissure support members 330, axial extensions 344, and posts 318, 320. As shown in FIG. 9, the prosthetic valve 300 can include a valvular structure 340, which is coupled to and supported inside the frame 302. The valvular structure 340 is configured to regulate the flow of blood through the prosthetic valve 300 from the inflow end 304 to the outflow end 306. The prosthetic valve 300 in this example can include the same features and function in a similar manner as described previously for prosthetic valve 100.

The valvular structure 340 can include a leaflet assembly comprising one or more leaflets 354 made of flexible material and having the same structural features as the leaflets 116 described herein. The leaflets 354 can be made from in whole or part, biological material, bio-compatible synthetic materials, or other such materials. Suitable biological material can include, for instance, bovine pericardium (or pericardium from other sources). Each leaflet 354 can have the same shape as leaflet 116 described above.

Each leaflet 354 of the valvular structure 340 can include a main body, an outflow edge portion 356, and the inflow edge portion 358. The inflow edge portion 358 of each leaflet 354 can comprise angled edge portions 362 and an apex edge portion 364. Each leaflet can also include one or more tabs located on opposite sides of the main body and outflow edge portion 356 of the leaflet 354 (for example, tabs 172, 174 in FIG. 4). Each leaflet can have axially extending side edge portions 360 extending between a tab and an angled edge portion 362. Each leaflet 354 of the valvular structure 340 can be secured to one another at their adjacent tabs to form respective leaflet commissures 338, each of which can be secured to a commissure support member 330 (as shown) and/or other portions of the frame 302. As shown in FIG. 9 for instance, the commissures 338 formed from adjacent tabs of adjacent leaflets 354 can be inserted axially between the first and second commissure arms 332, 334 and coupled to respective commissure support members 330, via sutures 366.

In the example depicted in FIGS. 9 and 10, the valvular structure 340 includes three leaflets 354 coupled to the frame 302 and arranged to collapse in a tricuspid arrangement. As illustrated in FIG. 9, certain portions of the inflow edge portion 358 of the leaflets 354 can be directly coupled to one or more support posts 320 and/or struts 308 of the frame 302 via sutures 368. The inflow edge portion 358 defines a generally scalloped shape edge that tracks the lower struts 370 of the frame 302 circumferentially around the frame.

As shown in FIGS. 9 and 10, the angled edge portions 362 of the leaflets 354 can be coupled to one or more of the lower struts 370 forming the first cells 310 and inflow apices 314. The angled edge portions 362 can largely track and align with the lower struts 370 extending between the inflow apices 314 and the support post 324. In the illustrated example of FIG. 10, the ends of the angled edge portions 362 nearest the apex edge portion 364, can be coupled to the lower ends of the lower struts 370 and/or to the posts 320 that extend between the major and minor vertices of respective first and second cells 310, 312. In having the angled edge portions 362 coupled to the lower struts 370 and/or post 320 of the inflow apices 314, the apex edge portions 364 are positioned at or substantially at the inflow end 304 of the frame 302. In some examples, the angled edge portions 362 of the leaflets can have a similar length to the lower struts 370. In such examples, the axial edge portions 360 can also largely track and have a similar length to the support posts 324 proximate and/or coupled thereto.

As shown in FIG. 9, the axial edge portions 360 of the leaflets 354 can extend along the support posts 324 disposed between adjacent first cells 310. As illustrated in FIGS. 9 and 10, the apex edge portions 364 of the leaflets 354 can extend between adjacent inflow apices 314 and be secured or anchored to the frame 302 via an axial extension 344 disposed between the inflow apices 314. In particular, the apex edge portion 364 of each leaflet extends between respective inflow apices 314 and is directly coupled to the frame 302 only at a corresponding axial extension 344 (for example, via sutures 372). As such, those portions of the apex edge portion 364 on either side of the axial extension 344 are unanchored and extend between the axial extension 344 and the posts 320 and/or lower struts 370 to which the leaflet is coupled. The apex edge portions 364 also span the circumferential gap between the adjacent inflow apices 314 formed by the lower struts 370 and support post 320. In some examples, the outer ends of the apex edge portion 364 near the angled edge portions 362 can be coupled to the posts 320 and/or lower struts 370 that form the inflow apices 314 such that a substantial portion of the apex edge portion 364 is still left unanchored to the frame 302.

Similar to the prosthetic valve 100, the apex edge portions 364 of the leaflets 354 can extend between pairs of adjacent inflow apices 314 such that a leaflet 354 extends between every other circumferential gap formed by the inflow apices 314 around the frame 302. Specifically, each two adjacent inflow apices 314 form a circumferential gap therebetween and each leaflet 354 can be coupled to a respective pair of inflow apices 314 such that a leaflet 354 extends between three of the six circumferential gaps at the inflow end 304 of the frame 302 in an alternating pattern. As such, a circumferential gap without a leaflet extending therebetween, can be said to extend between pairs of inflow apices 314 and between each of the apex edge portions 364 of the leaflets 354. In this case, one or more of the circumferential gaps not having a leaflet, can also include an axial extension 344 disposed between the inflow apices 314.

As previously mentioned, the leaflets 354 forming the valvular structure 340 can be made of a flexible material, and the axial extensions 344 made of a material that permits the extensions 344 to readily bend along their length or pivot. In this way, the apex edge portions 364 and axial extensions 344 are free to move radially inwardly and outwardly relative to an inner surface of the frame 302 during the working cycle of the prosthetic valve 300. In particular, the apex edge portions 364 and the free end 348 of the axial extensions 344 are configured to move radially inwardly toward a longitudinal axis of the frame 302 and move radially outwardly away from the longitudinal axis frame 302, such as when the axial extension moves back into a straight or partially straight configuration.

When the leaflets close under the back flow of blood, each axial extension 344 coupled to a leaflet 354 can bend along its length from its fixed end 346 to its free end 348 or deflect inwardly such that the apex edge portion 364 and free end 348 become radially spaced from the inner surface of the frame 102 a first distance. When the leaflets open under the forward flow of blood, each axial extension 344 can straighten or deflect outwardly such that the apex portion 364 and the free end 348 become radially spaced from the inner surface of the frame at a second distance, less than the first distance. In alternative examples, the axial extensions 344 can remain substantially straight and can pivot inwardly and outwardly at their fixed ends 346 as the leaflets cycle between their closed and open positions, respectively. The narrowed neck portions 352 promote flexing of the axial extensions at the fixed ends 346.

In some examples, the free ends 348 of the axial extensions 344 and apex edge portions 364 are always radially spaced from the inner surface of the frame 302 but are radially spaced from the frame 302 by varying degrees as the leaflets open and close, such as described. In other examples, the axial extensions 344 can move back to a straight or partially straight configuration such that the free ends 348 and apex edge portions 364 are radially aligned or substantially radially aligned with the inner or outer surface of the frame 302 when the leaflets are in an open configuration. In such examples, the axial extensions 344 in a straight configuration can prevent or reduce portions of the apex edge portions 364 and/or an outer skirt 374 from extending too far in the way of and obstructing blood flow across the valve 300 as blood enters the inflow end 304.

As shown in FIG. 10, each apex edge portion 364 of the leaflets 354 and/or axial extension 344 can also be coupled to one or more soft components of the prosthetic valve 300, including an outer skirt 374 mounted to the outer surface of the frame 302. As shown in FIG. 10, which shows a magnified side view of the inflow end 304 of valve 300, the outer skirt 374 can include an inflow end portion 376 positioned at the inflow end 304 of the frame 302 and an outflow end portion 378 positioned between the inflow end 304 and outflow end 306 of the frame 102. The outer skirt 374 can extend circumferentially around the outer surface of the frame 302 and extend axially from the inflow end portion 376 to the outflow end portion 378. The outer skirt 374 can be coupled to one or more interconnected struts 308, such as the lower struts that form the second cells 312 and/or any other strut of the frame 302.

As illustrated in FIG. 10, the inflow end portion 376 of the outer skirt 374 can be coupled to the apex edge portions 364 of the leaflets 354 and axial extensions 344. For instance, the inflow end portion 376 of the skirt 374 can be connected directly to the apex edge portion 364 extending between respective inflow apices 314 via sutures 380. The sutures 380 can form whip stitches that extend through the apex edge portions 364 of the leaflets (and/or a connecting skirt connected to the apex edge portions 364) and the inflow end portion 376 of the skirt and around the inflow edges of the apex edge portions and the inflow end of the skirt. The sutures 380 also can extend around the axial extensions 344 or through the apertures 350 of the axial extensions to connect the skirt to the axial extensions 344. In lieu of or in addition to sutures 380, one or more other fasteners (for example, pins, screws, etc.) can be used to secure the skirt 374 to the apex portions of the leaflets and/or the axial extensions. In some examples, the outer skirt 374 can also be connected to one or more struts forming the second cells 312 via sutures 388. The outer skirt 374 is configured to establish a seal against the surrounding native annulus to prevent or minimize paravalvular leakage and to prevent retrograde blood between the leaflets and the frame from flowing outwardly through the cells of the frame. Notably, the inflow end portion 376 of the outer skirt 374 can move radially inwardly and outwardly with the axial extensions 344 and the apex edge portions 364 of the leaflets 354 during working cycles of the prosthetic valve.

Advantageously, the axial extensions 344 allow the apex edge portions of the leaflets to participate in the coaptation of the leaflets by allowing the free edges of the leaflets to move closer toward one another during valve closure yet provide sufficient support to the apex portions of the leaflets and the outer skirt to prevent excessive inward movement of the apex portions of the leaflets and the outer skirt that could otherwise impair the performance of the leaflets (such as illustrated in FIG. 7).

In alternative examples, the axial extensions 344 can also be used to support an inner skirt (not shown) that is connected to the inner surface of the frame 302, in addition to the outer skirt 374, so that the inner skirt, the outer skirt, and the apex edge portions of the leaflets can move inwardly and outwardly during working cycles of the prosthetic valve. In some examples, the prosthetic valve 300 can have an inner skirt supported by the axial extensions 344 and the outer skirt can be omitted. In some examples, the prosthetic valve 300 can have an inner skirt and/or an outer skirt that are not secured to the axial extensions by sutures or other fasteners.

As shown in FIG. 8, in some examples, the axial extension 344 can be disposed between one or more pairs of adjacent inflow apices 314 without a leaflet extending therebetween (FIG. 8). In this instance, those axial extensions 344 not coupled to a leaflet can be configured to prevent or mitigate the portions of the outer skirt 374 connected to those axial extensions 344 from moving radially inwardly into the valve during working cycles of the prosthetic valve.

Referring to FIGS. 9 and 10, and as previously mentioned, the first and second posts 318, 320 can be functionally similar to posts 142 to radially expand and/or compress the frame 302 in conjunction with actuator members received within the posts 318, 320. The posts 318, 320 can be axially aligned with one another and comprise an inner bore extending along a length of the posts and through which an actuator member, such as the illustrated threaded rod 382, can extend. An outflow end portion 384 of each second post 320 can include or house a nut 386. As shown in FIGS. 9 and 10, the nut 386 can be visible through a window in the outflow end portion 384. The nut 386 can include an inner threaded bore configured to engage the threads of the threaded rod 382 such that rotation of the threaded rod 382 causes the second post 320 coupled to the nut 386 to move relative to the first post 318, which can be held stationary against a delivery apparatus, as further described below.

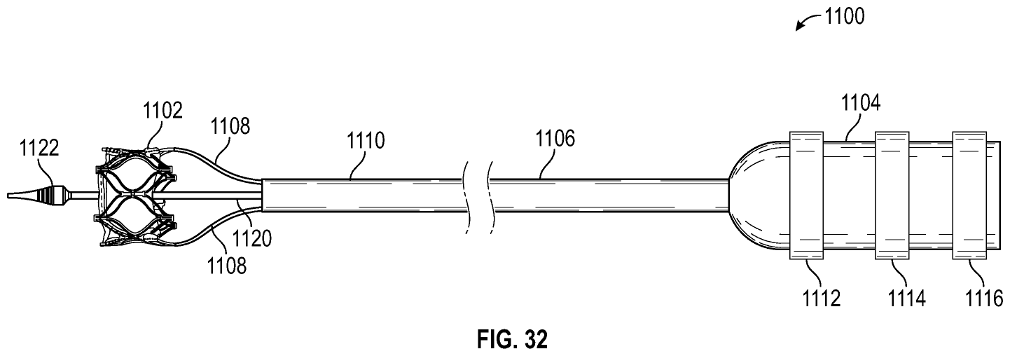

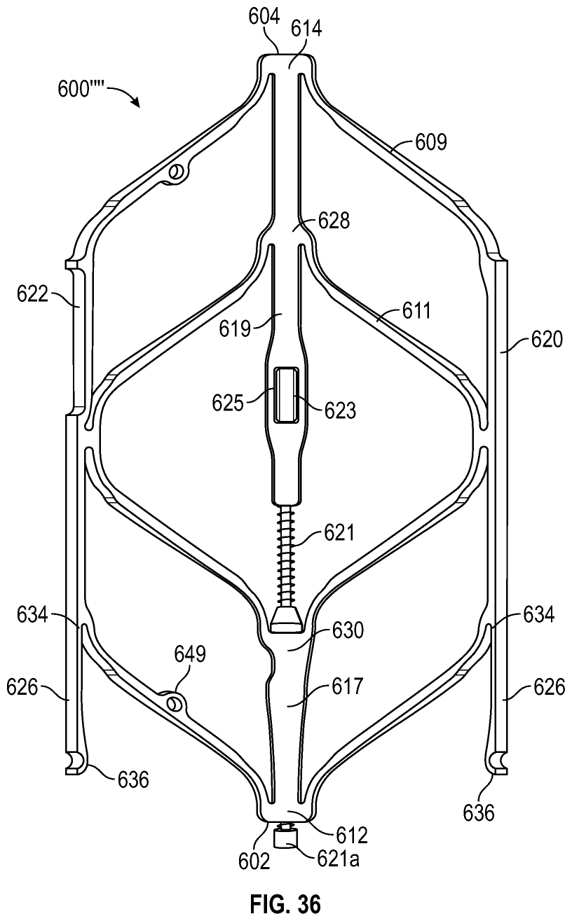

The actuator members 382 can include head portions 382a adjacent outflow apices 316 of the frame 302. A stopper 383 can be mounted on each actuator member 382 between the posts 318, 320. The head portions 382a can be configured to form a releasable connection with respective actuation assemblies (for example, actuation assemblies 1108 of FIG. 32) of a delivery apparatus. The actuation assemblies of the delivery apparatus can include rotatable actuators that transmit rotation to the actuator members 382 of the prosthetic valve, thereby radially expanding or radially compressing the prosthetic valve. Coupling the actuation assemblies of the delivery apparatus to the head portions 382a at the outflow end of the frame can be advantageous when delivering the prosthetic valve to the native aortic valve through the aorta in a retrograde approach. In other examples, such as shown in FIG. 36, the head portions 382a of the actuator members can be positioned at the inflow end of the frame for coupling to the actuation assemblies of a delivery apparatus, such as for delivering the prosthetic valve to the native aortic valve in a transapical delivery approach, or delivering the prosthetic valve to the native mitral valve in a trans-septal delivery approach.

In other examples, in addition to or in lieu of using a nut, a portion or the entirety of the inner bore of the second posts 320 can be threaded. For example, an outflow end portion of the second post 320 can comprise inner threads configured to engage the threaded rod 382 such that rotation of the threaded rod 382 causes the second post 320 to move relative to the first post 318.

Rotation of the threaded rod 382 in a first direction (for example, clockwise) can cause corresponding axial movement of the second post 320 toward the first post 318, thereby expanding the frame 302. When the threaded rod 382 is rotated to radially expand the frame, the head portion 382a can bear against the outflow apex 316 and apply a distally directed force against the post 318, while the treaded connection between the rod 382 and the nut 386 generates a proximally directed force applied to the post 320, which moves the post 320 toward the post 318. Rotation of the threaded rod 382 in a second direction (for example, counterclockwise) causes corresponding axial movement of the second post 320 away from the first post 318, thereby radially compressing the frame 302. When the threaded rod is rotated to radially compress the frame, the stopper 383 can bear against an adjacent end of the first post 318 to apply a proximally directed force to the post 318, while the threaded connection between the rod 382 and the nut 386 generates a distally directed force applied to the post 320, which moves the post 320 away from the post 318. As the frame 302 moves from a radially compressed configuration to a radially expanded configuration, the axial spacing between the first and second posts 318, 320 decreases. As the frame 302 moves from a radially expanded configuration to a radially compressed configuration, the axial spacing between the first and second posts 318, 320 increases.

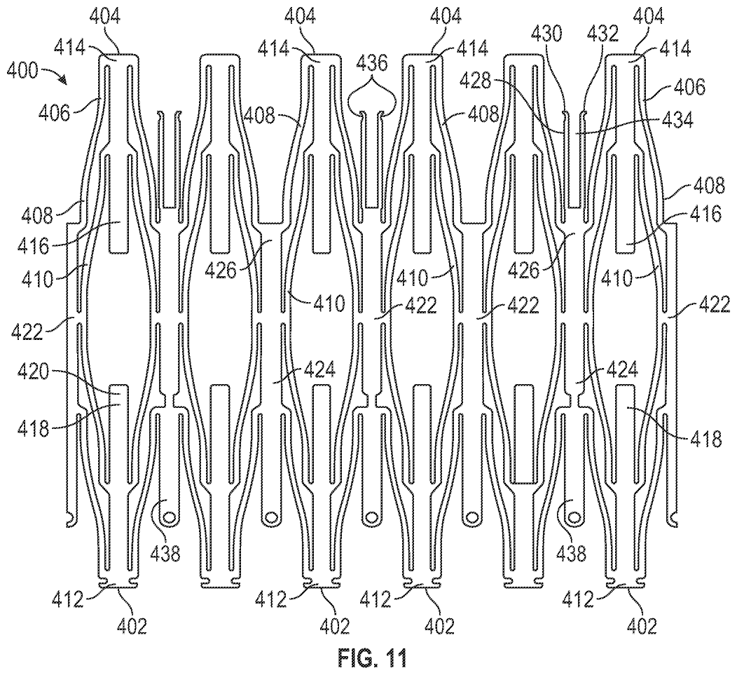

FIG. 11 illustrates another example of a frame 400 for a prosthetic heart valve (for example, valve 100) in a compressed and flattened state. The frame 400 can be structurally similar to and function in a similar manner to frame 102 and frame 302. For instance, the frame 400 includes an inflow end 402, an outflow end 404, and a plurality of circumferentially extending rows of interconnected struts 406 forming a plurality of first and second elliptical-shaped cells 408, 410 and a plurality of inflow and outflow apices 412, 414 at the inflow end 402 and outflow end 404, respectively. The frame 400 in this example can also include a plurality of axially extending posts, including pairs of first and second posts 416, 418 interconnecting respective first and second cells 408, 410, and support posts 422 disposed between adjacent cells 408, some of which posts 422 can include respective commissure support members 428.

As shown in the illustrated example of FIG. 11, the frame 400 comprises six first cells 408 that can extend circumferentially in a row, with a second cell 410 within each first cell. The frame can also include six pairs of posts 416, 418 coupled to respective pair of cells 408, 410, six support posts 422, six axial extensions 438, and three commissure support members 428. In other examples, however, the frame 400 can comprise a greater or fewer number each of these features listed.

The support posts 422 of the frame 400 can extend longitudinally and have an inflow end portion 424 and an outflow end portion 426. The outflow end portion 426 of one or more support posts 422 can include a commissure support member 428, structurally and functionally similar to commissure support members 330 described herein. For instance, the commissure support members 428 can include first and second commissure arms 430, 432 defining a commissure opening 434 between them. The commissure opening 434 can extend radially through a thickness of the post 422 and at the outflow end portion 426 of commissure support member 428 such that the commissure support member 428 is configured to receive a leaflet commissure (for example, commissures 118, 338). In this instance, a commissure formed of a pair of adjacent leaflets of a valvular structure can slide axially between the first and second commissure arms 430, 432 and into the opening 434 as to mount and support the valvular structure within the frame 400 when constructing the prosthetic valve. In some examples, the opening 434 can be fully enclosed by the post 422 (for example, the commissure support member 120 of FIG. 3) such that a valvular structure can be slid radially, rather than axially, into the commissure opening 434.

In the illustrated example, the first and second commissure arms 430, 432 can each include a respective notch or indentation 436. Each indentation 436 can be located along each arm 430, 432 proximate the outflow end 404 of the frame 400. Each indentation 436 can be configured to receive one or more fasteners (for example, sutures) that extend from the indentation 436 of the first commissure arm 430 to the indentation 436 of the second commissure arm 432 (and vice versa). The combination of the indentations 436 and one or more fasteners can form a boundary at or near the indentations 436 to prevent or limit axial movement of the leaflet commissure between the first and second commissure arms 430, 432 toward the outflow end 404 of the frame 400. The combination of the indentations 436 and one or more fasteners can also act to apply a lateral force to the sides of the leaflet commissure via the first and second commissure arms 430, 432 to secure the leaflet commissure within the opening 434 of the commissure support member 428.

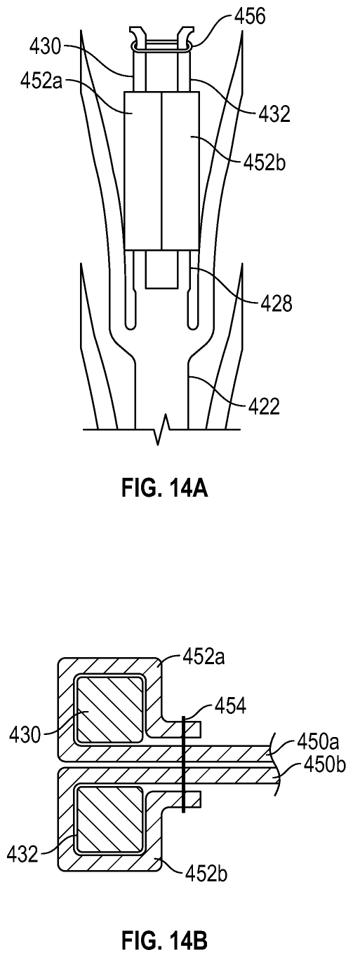

FIGS. 14A and 14B show one example for mounting a commissure of a leaflet assembly to a commissure support member 428. As shown, first and second leaflets 450a, 450b having respective commissure tabs 452a, 452b that extend radially through the space between arms 430 and 432. The commissure tab 452a can be wrapped around the arm 430 such that an end of the tab is near or against the main body of the leaflet 450a inside the frame. The commissure tab 452b can be wrapped around the arm 432 such that an end of the tab is near or against the main body of the leaflet 450b inside the frame. One or more sutures 454 can be stitched through the tabs 452a, 452b and the main bodies of the leaflets 450a, 450b to secure the commissure tabs in place. A flexible member 456 (for example, a suture, wire, yarn, cable, etc.) can be wrapped around the arms 430, 432 within the indentations 436. The flexible member 456 can be tightened around the arms 430, 432 so as to force the arms closer together and apply a compressive force against the commissure tabs 452a, 452b, which helps retain the commissure tabs in place on the arms 430, 432.

It should be understood that FIGS. 14A and 14B show one specific technique for folding the commissure tabs and securing them to the arms 430, 432. However, various other techniques can be used. For example, U.S. Pat. No. 9,393,110, which is incorporated herein by reference, discloses various methods of folding commissure tabs and mounting them to a commissure support member defining a commissure window. The methods and techniques disclosed in the '110 patent alternatively can be used to mount the leaflets 450a, 450b to the commissure support member 428. The methods and techniques described above and those disclosed in the '110 patent also can be used to mount the commissures of a leaflet assembly to the commissure support members of a frame of any of the prosthetic valves disclosed herein.

One or more of the plurality of axially extending struts or posts 416, 418 shown in FIG. 11 can also be configured to receive actuator members and/or serve as stoppers to prevent over expansion of the frame 400. As illustrated in FIG. 11, the frame 400 can include a plurality of first axial posts 416 extending from and coupled to the outflow apices 414 at the outflow end 404, and a plurality of second axial posts 418 extending from and coupled to the inflow apices 412 at the inflow end 402. Each axial post 416, 418 can extend from a respective outflow or inflow apex 414, 412 and into the opening formed by the first and second cells 408, 410. Each first axial post 416 can be axially aligned with a corresponding second axial post 418 to form a pair of first and second axial posts.

Each pair of axial posts 416, 418 can be configured to receive a respective actuator member to radially expand and/or compress the frame 400, as previously described above for prosthetic valves 100, 300. For example, each axial post 416, 418 can comprise an inner bore (not shown) extending along a length of the post 416, 418 through which an actuator member (for example, rod 158 or threaded rod 382) can extend. Each bore extending through its respective post 416, 418 can, for instance, be configured to engage the actuator member such that rotation of the actuator member causes the second post 418 to move axially relative to the first post 416. For example, rotation of the actuator member in a first direction (for example, clockwise) causes corresponding axial movement of the first and second axial posts 416, 418 toward one another, expanding the frame 400. In a like manner, rotation of the actuator member in a second direction (for example, counterclockwise) causes corresponding axial movement of the first and second axial posts 416, 418 away from one another to compress the frame 400. In alternative examples, the actuator members can be configured to be pulled or slid axially (rather than rotated) to move the second posts 418 toward the first posts 416 to radially expand the frame. In such examples, the actuator members can be rods, tethers, sutures, cables, wires, and the like.

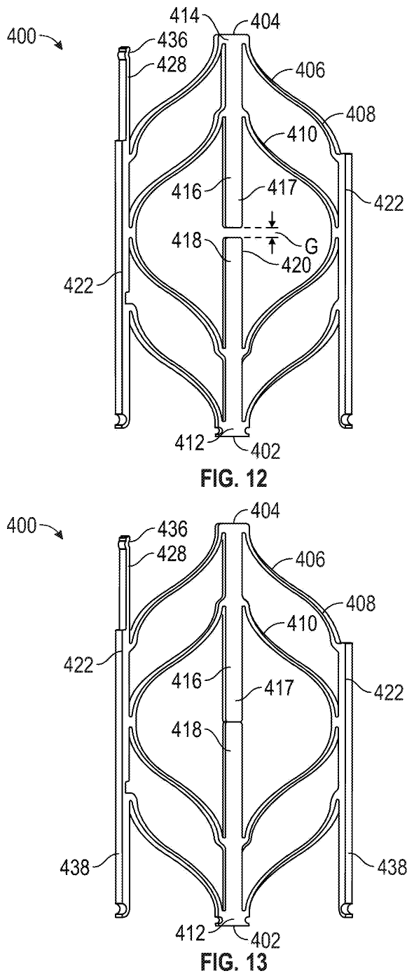

Referring now to FIGS. 12 and 13, in addition to or in lieu of being configured to receive actuator members, the first and second axial posts 416, 418 can be configured as a stopper to prevent over-expansion of the frame 400. For the purpose of illustration and to facilitate discussion, FIGS. 12 and 13 illustrate a single first cell 408 and corresponding second cell 410 of the frame 400. While only single first and second cells 408, 410 of the frame 400 are depicted in FIGS. 12 and 13, it should be appreciated that the frame 400 forms an annular structure.

FIGS. 12 and 13 show first and second cells 408, 410 forming respective inflow and outflow apices 412, 414. The first and second cells 408, 410 are located between a pair of adjacent support posts 422, which may include axial extensions 438 (for example, similar to axial extensions 344) with one support post 422 including a commissure support member 428. FIGS. 12 and 13 show the first and second cells 408, 410 in a radially expanded configuration. The frame 400 as depicted in FIG. 12 is considered to be in a partially expanded configuration, while the expanded configuration of the frame 400 depicted in FIG. 13 is considered to be in a maximum or fully expanded configuration. The first posts 416 have inflow end portions or extensions 417 that extend into the second cells 410 toward the second posts 418. The second posts 418 have outflow end portions or extensions 420 that extend into the second cells 410 toward the first posts 416.

As shown in FIG. 12, the outflow end (the upper end in the drawings) of each second axial post 418 and the inflow end (the lower end in the drawings) of each corresponding first axial post 416 can be separated by an axial gap G when the frame 400 is in a radially compressed or partially expanded configuration. The axial gap G provides enough axial spacing for the posts 416, 418 to move toward and/or away from each other during radial expansion and radial compression of the frame 400, respectively. For instance, the gap G decreases or narrows as the frame 400 is radially expanded and increases or widens as the frame is radially compressed. This narrowing and widening of the axial gap G also corresponds to an increase and decrease in the outer diameter of the frame 400, respectively, during radial expansion and compression of the frame.

As shown in FIG. 13, the outflow end of the second axial post 418 and the inflow end of the first axial post 416 can abut or contact one another once the frame 400 is expanded to a fully expanded configuration. For instance, if the frame 400 is continually radially expanded, the axial gap G narrows and the ends of the posts 416, 418 eventually contact one another. This contact between the first and second axial posts 416, 418 prevent further axial movement of the struts 406 relative to one another, thereby preventing any further expansion of the frame 400. In this manner, when the outflow and inflow ends of the posts 416, 418 are in contact with one another, the axial posts 416, 418 act as a stopper to prevent over-expansion of the frame 400 and the frame is said to be in a maximum radially expanded configuration.

In some examples, the length of each first and second axial post 416, 418, and more specifically, the lengths of the extensions 417 and the extensions 420, can be altered to modify the maximum or full radial expansion of the frame 400. For example, the length of the inflow extensions of the first posts 416, and the length of the outflow extensions of the second posts 418, can be lengthened and/or shortened to decrease or increase the degree to which frame can radially expand, respectively. In particular, the first and second axial posts 416, 418 as shown in FIGS. 12 and 13, are equal or substantially equal in length. Accordingly, an increase in the length of one or both axial posts 416, 418 can cause the posts to contact one another with less radial expansion than that radial expansion depicted in FIGS. 12 and 13. Inversely, a decrease in the length of the one or both axial posts 416, 418 can require greater radial expansion beyond that of the radial expansion depicted in FIGS. 12 and 13. Although the posts 416, 418 can have equal or substantially equal lengths, in other examples, one of the first and second posts can have a length greater than the other of the first and second posts.

In examples where the first and second axial posts 416, 418 of frame 400 are configured to receive actuator members and serve as a stopper, the length of the posts 416, 418 can prolong the inner bores extending through the posts. This, among other things, can limit the length of an actuator member, such as a rod (for example, rod 158 and threaded rod 382), exposed between respective inflow and outflow apices 412, 414 of the frame 400. For instance, an actuator member's exposure can be limited to the axial spacing between the upper end of the second post 418 and the lower end of the first post 416, thereby preventing or reducing buckling of the actuator member during radially expansion or compression of the frame. In examples where the actuator members comprise flexible pull wires and/or sutures, the axial posts 416, 418 can reduce or eliminate misalignment between corresponding inflow and outflow apices because of the extended length of the respective bores.

As shown in FIGS. 12 and 13, the axial posts 416, 418 extend axially into the opening formed by the first and second cells 408, 410. In this way, the first axial post 416 and/or the second axial post 418 can provide an additional support structure to an outer skirt (for example, outer skirt 128 or outer skirt 374) coupled to the outer surface of the frame 400. As such, the posts 416 and/or the posts 418 can be configured to prevent an outer skirt from protruding inwardly through the opening of the first and/or second cells 408, 410, which can cause disruption to blood flow and an unwanted increase in pressure gradients across the valve. Also, the outer skirt can be connected to one or both of the posts 416, 418 where the posts extend into the inner cell 410. For example, in one implementation, the outer skirt can extend from the inflow end of the frame to a location between the inflow and outflow ends of the frame, typically covering at least half of the length of the frame. As shown in FIGS. 1 and 10, the outer skirt can extend from the inflow end of the frame to a plane bisecting the inner cells. In such a configuration, the extensions 420 of the second posts 418 can block the outer skirt from protruding inwardly through the cells 410.

Moreover, where there are relatively large open spaces in the middle of the frame, such as with the frame of FIGS. 1-3, the actuator members can apply greater forces at the distal end of the frame (the end farthest from the delivery apparatus; the inflow end in the illustrated example) than the proximal end of the frame (the end closest to the delivery apparatus; the outflow end in the illustrated example). The unequal forces can create a moment that causes distal end of the frame to expand at a greater rate than the proximal end of the frame, causing the frame to expand in an uneven and non-cylindrical manner, resulting in bending of the frame during radial expansion. Advantageously, the extensions 417, 420 of posts 416, 418 reduce the amount of open space in the middle section of the frame between the adjacent ends of the posts, such as compared to the frame of FIG. 1. This provides additional support to the actuator members (for example, members 158 or 382) and better distributes the forces of the actuator members along the length of the frame and away from the opposing ends of the frame, thereby causing the opposing ends of the frame to expand at the same or substantially the rate. The frame therefore expands cylindrically in an even and predictable manner without bending.

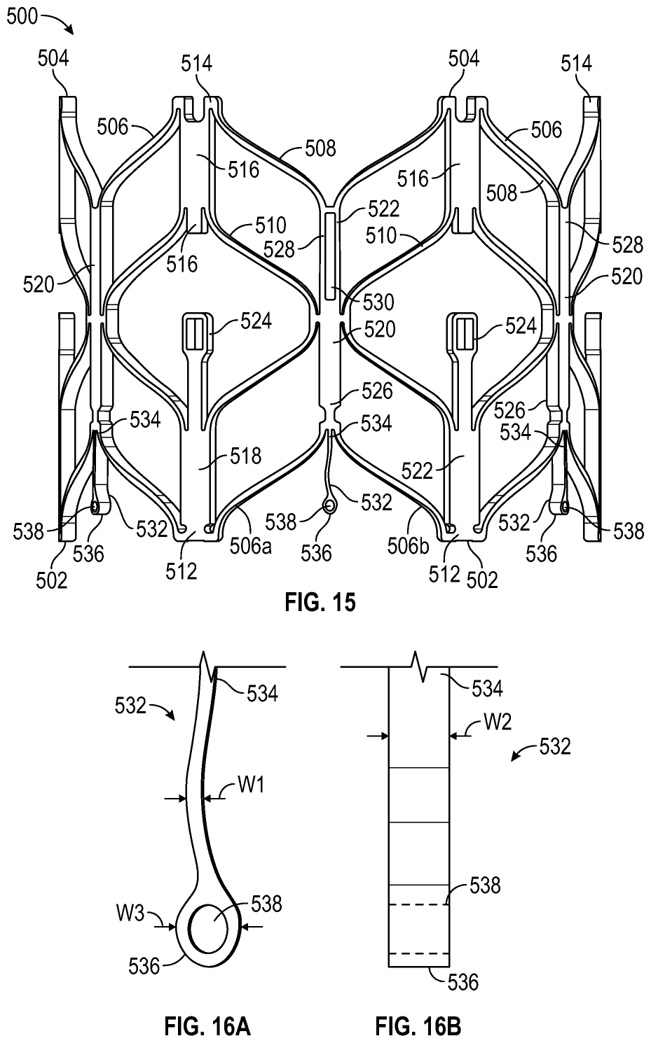

FIG. 15 illustrates another example of a frame 500 for a prosthetic heart valve in a radially expanded configuration. While only one side of the frame 500 is shown in FIG. 15, it should be appreciated that the frame 500 forms an annular structure. The frame 500 can be structurally similar to and function in a similar manner to frame 102, frame 302, and frame 400. The frame 500, for instance, includes an inflow end 502, an outflow end 504, and a plurality of circumferentially extending rows of interconnected struts 506. The rows of struts 506 form a plurality of first and second elliptical-shaped cells 508, 510 and a plurality of inflow and outflow apices 512, 514 at the inflow end 502 and outflow end 504, respectively. The frame 500 can also include a plurality of axially extending posts, including pairs of first and second posts 516, 518 interconnecting respective first and second cells 508, 510, as well as support posts 520 disposed between adjacent cells 508, some of which can include respective commissure support members 522.

The pairs of first and second posts 516, 518 can be functionally similar to posts 142 (FIGS. 1-3), posts 318, 320 (FIGS. 8-10), and posts 416, 418 (FIGS. 11-13) such that the posts 516, 518 are configured to radially expand and/or compress the frame 500. The posts 516, 518 can, for instance, be axially aligned with one another and each post 318, 320 can comprise an inner bore extending along a length of the posts and through which an actuator member, such as a threaded rod (for example, threaded rod 382) can extend. To engage the threads of a threaded rod, for example, an outflow end portion 524 of each second post 518 can also comprise inner threads along a portion or the entirety of its respective inner bore and/or can house a nut having a threaded bore (for example, FIGS. 9-10) to mate with a respective threaded rod. In this instance and as described above, rotation of a threaded rod in a first direction (for example, clockwise) can cause corresponding axial movement of the of the first second posts 516, 518 toward one another, expanding the frame 500. Similarly, rotation of a threaded rod in a second direction (for example, counterclockwise) causes corresponding axial movement of the first and second posts 516, 518 away from one another, compressing the frame 500.

In alternative examples, the actuator members can be configured to be pulled or slid axially (rather than rotated) to move the first and second posts 516, 518 toward and away from one another to radially expand and compress the frame. In such examples, the actuator members can be rods, tethers, sutures, cables, wires, and the like.

Each support post 520 can extend longitudinally and have an inflow end portion 526 and an outflow end portion 528. As illustrated in FIG. 15, the outflow end portion 528 of one or more support posts 520 can include a commissure support member 522 within (or extending from) the body of its respective support post. The commissure support member 522 can, for instance, comprise a window 530 formed by the support post 520 that fully encloses or frames an opening extending radially through the thickness of the support post 520. Each window 530 of the commissure support members 522 can be configured to receive a pair of leaflet commissures formed by adjacent leaflets when assembling the prosthetic valve in order to mount a valvular structure within the frame 500.

In other examples, the commissure opening can have a variety of shapes, such as square, oval, square-oval, triangular, L-shaped, T-shaped, C-shaped, etc. In some examples, the opening is not fully enclosed, and the leaflet commissures can be slid axially within the opening of the commissure support (for example, FIGS. 11 and 14A-14B).

The inflow end portion 526 of each support post 520 can also include a cantilevered strut or axial extension 532 that extends toward the inflow end 502 of the frame 500. In the illustrated example, each axial extension 532 can include a fixed end 534 coupled to a respective support post and a free end 536 that extends toward the inflow end 502 of the frame 500. The axial extensions 532 can have a length such that upon radial expansion of the frame 500, the free ends 536 are axially spaced from the inflow end of the frame 500. In some examples, however, the free ends 348 can align with or be located proximate the inflow end of the frame 500 upon the frame's expansion. As shown in FIG. 15, each axial extension 532 is disposed midway between adjacent inflow apices 512 and between a pair of lower struts 506a, 506b forming one half of the lower portion of respective adjacent first cells 508.

Turning now to FIGS. 16A-16B, FIG. 16A shows a magnified view of the circumferential outer surface of an axial extension 532, while FIG. 16B shows the side profile of the axial extension 532 from FIG. 16A. As shown in FIGS. 16A-16B, each axial extension 532 can have a first thickness or width W1 in the circumferential direction (that is, in a direction toward respective adjacent inflow apices 512) and a second thickness or width W2 in the radial direction (that is, extending between the inner and outer surfaces of the frame 500). FIG. 16A shows each axial extension 532 can have a relatively narrow circumferential first width W1 along much of its length. More specifically, the axial extensions 532 can have a narrow width along the length of the extensions from the fixed ends 534 to the segment of the axial extensions 532 where the edges of the extensions begin to widen to a circumferential third width W3 to accommodate an opening or aperture 538. Accordingly, the free ends 536 of the axial extensions 532 can be said to have a circumferential third width W3 relatively wider or greater than the circumferential width W1 of the fixed ends 534 and remaining portions of the axial extensions 532. In some examples, the circumferential first width W1 of the axial extensions 532 can vary (for example, increase and decrease) along the length of the axial extensions while still being relatively narrower in width in relation to the circumferential third width W3 and/or radial second width W2 as described herein.

FIG. 16B shows the radial second width W2 of the axial extensions is comparatively wider or greater than the circumferential first width W1. By extension, the circumferential first width W1 of the axial extensions 532 is comparatively narrower or less than the radial second width W2. This relative difference between the first width W1 and the second width W2 of the axial extensions 532 allows the axial extensions to deflect laterally or in a circumferential direction but maintain relative stiffness in a radial direction. Specifically, the comparatively narrow circumferential first width W1 of the axial extensions 532 provides the extensions with enough flexibility or bendability such that the axial extensions 532 are configured to deflect laterally in either direction when axial forces act on the free ends 536. As such, each axial extension 532 is capable of moving in the direction of either of the adjacent inflow apices 512 the axial extension is disposed between depending on the force applied to the axial extension.

This lateral or circumferential movement of the axial extensions 532 allows, for instance, the axial extensions to move to one side, or from one side to the other, if native tissue contacts and applies an axial force on the free ends 536 during delivery of the prosthetic valve through a patient's vasculature. As an example, as the prosthetic heart valve is advanced through the native lumen and/or manipulated within the native valve, the axial extensions 532 are configured to yield or give way to forces that may be applied to the axial extensions by the native tissue, thereby allowing the prosthetic heart valve to be advanced in an atraumatic fashion. Moreover, the free end 536 can also have a rounded outer edge to form an atraumatic apex as to reduce potential injury to the native tissue if and when contact does occur.

Conversely, the comparatively wider, radial second width W2 of the axial extensions 532 cause the extensions to be relatively rigid in the radially inward direction in comparison to the deflection of the axial extensions 532 in the lateral directions. In particular, the axial extensions 532 of the example shown in FIGS. 16A-16B are configured by way of the radial second width W2 to be relatively resistant to radially inward motion (for example, toward a longitudinal axis of the frame 500) while maintaining the lateral mobility. The second width W2 in this way, can provide adequate rigidity and support to retain soft components of the prosthetic heart valve, such as an outer skirt, on the outer surface of the frame 500 (for example, FIG. 18). This retention of the soft components, for example, can prevent the soft components from protruding radially inward into openings of the frame 500 during operation of the prosthetic heart valve, which can cause an undesired increase in the pressure gradient across the valve if protrusion does occur.

Each axial extension 532 can be formed of a variety of suitable materials, such as stainless steel, a cobalt chromium alloy, or a nickel titanium alloy (“NitTi”), including Nitinol. In specific examples, each axial extension 532 can be formed of a material with shape memory properties, such as Nitinol. Being made of such materials can, for instance, allow the axial extensions 532 to deflect laterally in the direction of either adjacent inflow apex 512 more readily than if the axial extension 532 were made of relatively more rigid material.

FIG. 16A also shows the axial extensions 532 can have an asymmetrical outer surface. For instance, the axial extensions 532 can have an asymmetrical curvature with respect to a central longitudinal line bisecting the axial extensions (for example, from the free ends 536 to the fixed ends 534). In some examples, this asymmetry or curvature can allow the free ends 536 of the axial extensions 532 to move axially in the direction of the outflow end 504. For instance, one or more bends along the length of the axial extensions 532 can allow the axial extensions to undergo a degree of axial compression when in contact with native tissue such that the length between the outer most inflow end of the free ends 536 and respective support post 520 is shortened or decreased.

As mentioned, the free ends 536 of the axial extensions 532 can also include an opening or aperture 538 extending radially through the thickness of the extension. The apertures 538 can be sized and shaped to receive one or more fasteners (for example, sutures) and/or soft components of the prosthetic valve (for example, an outer skirt). For instance, as shown in FIGS. 16A-16B, the apertures 538 can be circular in shape and sized to receive a suture extending therethrough. However, in some examples, the apertures 538 can be rectangular in shape or any other suitable shape as to be configured to receive one or more sutures or other fasteners.

As shown in FIG. 16B, the axial extensions 532 can have a linear or substantially linear side profile along the length of the axial extensions. Stated differently, the radial second width W2 of the axial extensions 532 can be constant along a substantial portion and/or the entire length of the axial extensions. In some examples, however, the radial width can vary along the length of the axial extensions such that one portion of axial extensions has relatively narrow radial width.

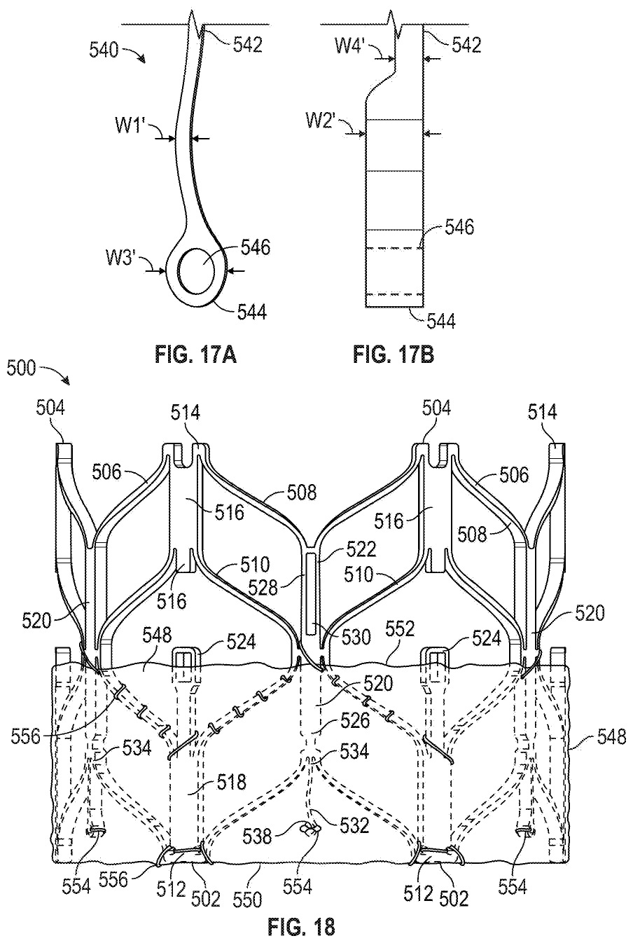

FIGS. 17A-17B show an axial extension 540 according to another example. In particular, the cantilevered strut or axial extension 540 shown in FIGS. 17A-17B can comprise a fixed end 542 coupled to a respective support post and a rounded free end 544, which extends toward the inflow end 502 of the frame 500 and includes an aperture 546 extending therethrough. In a similar fashion to the axial extensions 532, each axial extension 540 can be disposed between a pair of adjacent inflow apices 512 and have a circumferential first width W1′, which is relatively narrower or smaller than a radial second width W2′ and a circumferential third width W3′ of the axial extensions 540.

One difference between the axial extensions 540 and the axial extensions 532 is that the fixed ends 542 of the axial extensions 540 include a radial fourth width W4′ which is relatively narrower than the radial second width W2′. In this manner, the axial extensions 540 are configured to be functionally similar to axial extensions 344 (FIGS. 8-10) and axial extensions 438 (FIGS. 11-13). That is, the relatively narrow fourth width W4′ of the fixed end 542 provides the axial extension 540 with enough flexibility or bendability to allow the extension to bend or curve radially between its fixed end 542 and its free end 544 such that the free end 544 can bend radially inwardly into the frame 500 (for example, toward a longitudinal axis of the frame 500). In this way, the axial extensions 540 can be coupled to the inflow edge of corresponding leaflets (for example, leaflets 116) of the prosthetic heart valve in such a way as to allow both the inflow edge of the leaflet and axial extension 540 to move radially inwardly into the frame 500 during the working cycle of the prosthetic valve, as described above.

Moreover, as shown in FIG. 18, each axial extension 532 and/or axial extension 540 can be coupled to one or more soft components of the prosthetic heart valve, including an outer skirt 548 mounted to the outer surface of the frame 500. The outer skirt 548 can include an inflow end portion 550 positioned at the inflow end 502 of the frame 500 and an outflow end portion 552 positioned between the inflow end 502 and outflow end 504 of the frame 500. The outer skirt 548 can extend circumferentially around the outer surface of the frame 500 and extend axially from the inflow end 502 to the outflow end 504. The outer skirt 548 can be coupled to one or more interconnected struts 506, such as the lower struts that form the first and second cells 508, 510 and/or any other strut of the frame 500 via sutures 556. The inflow end portion 550 of the outer skirt 548 can also be coupled to the axial extensions 532 and/or axial extensions 540 via sutures 554. The sutures 554, for instance, can form stitching that extends around the axial extensions 532, 540 and/or through the apertures 538, 546. In some examples, in lieu of or in addition to sutures 554, one or more other fasteners, such as pins or screws, can be used to secure the outer skirt 548 to the axial extensions 532, 540.

In some examples, the outer skirt 548 can be coupled to an inflow edge portion (for example, apex edge portion) of one or more leaflets using whip stitching that extends through the inflow edge portion of the leaflets and the inflow end portion 550 of the skirt 548 and around the inflow edge portion of the leaflets and the inflow edge of the skirt 548. This, among other things, allows the inflow end portion 550 of the skirt 548 to move laterally and/or radially inwardly with the axial extensions 532 and/or axials extensions 540, and the inflow edge portion of the leaflets during working cycles of the prosthetic valve. The outer skirt 548 in this configured can, for instance, establish a seal against the surrounding native annulus to prevent or minimize paravalvular leakage and to prevent retrograde blood between the leaflets and the frame from flowing outwardly through the cells of the frame.

As depicted in FIGS. 15 and 18, the frame 500 comprises six first cells 508 that can extend circumferentially in a row, with a second cell 510 within each first cell. The frame can also include six pairs of posts 516, 518 coupled to respective pair of cells 508, 510, six support posts 520, six axial extensions 532 (or alternatively, axial extensions 540), and three commissure support members 522. In other examples, however, the frame 500 can comprise a greater or fewer number each of these features listed. In some examples, the frame 500 can also comprise any combination of axial extensions 532 and axial extensions 540. For instance, the frame 500 can include three axial extensions 532 and three axial extensions 540 such that three axial extensions are configured to only deflect laterally and three axial extensions are configured to deflect both laterally and radially inwardly.

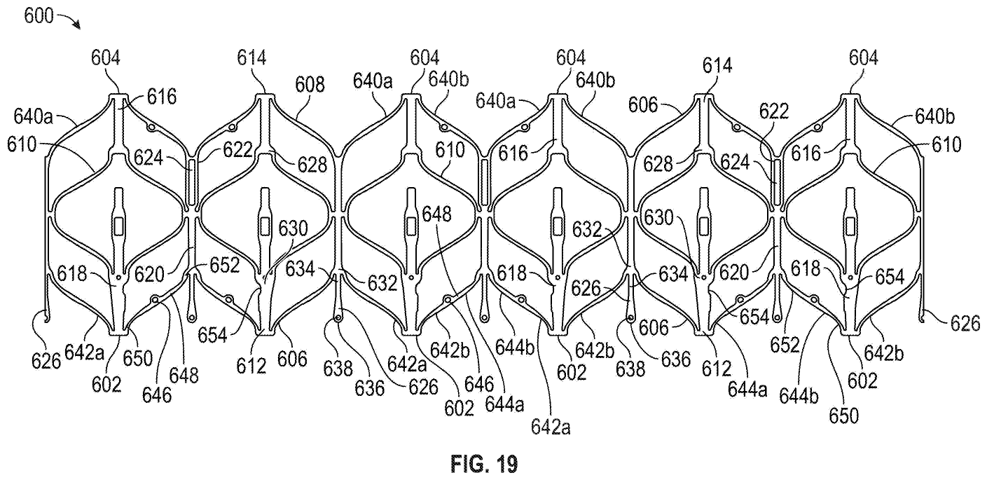

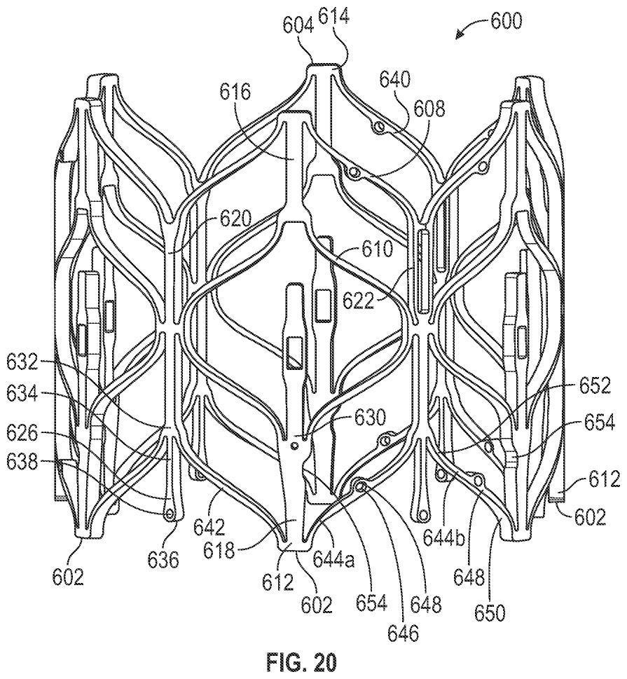

FIGS. 19 and 20 depict another example of a frame 600 for a prosthetic heart valve (for example, valve 100). FIG. 19 shows the frame 600 in an expanded and flattened state while FIG. 20 shows the frame 600 in a radially expanded state. The frame 600 can be structurally similar to and function in a similar manner as frame 102, frame 302, frame 400, and frame 500. For example, the frame 600 includes an inflow end 602, an outflow end 604, and a plurality of circumferentially extending rows of interconnected struts 606 forming a plurality of first and second elliptical-shaped cells 608, 610 and a plurality of inflow and outflow apices 612, 614 at the inflow end 602 and outflow end 604, respectively. The frame 600 also includes a plurality of axially extending posts, including pairs of first and second posts 616, 618 interconnecting respective first and second cells 608, 610, and support posts 620 disposed between adjacent cells 608. Some of the supports posts 620 can include respective commissure support members 622 which can be similar to commissure support members 522 such that commissure support members 622 can comprise a window 624 formed by the support post 620 that fully encloses or frames an opening extending radially through the thickness of the support post.

The frame 600 can include a plurality of first axial posts 616 extending from and coupled to the outflow apices 614 at the outflow end 604, and a plurality of second axial posts 618 extending from and coupled to the inflow apices 612 at the inflow end 602. Each first axial post 616 can extend between a respective outflow apex 614 of the frame 600 and an outflow apex 628 disposed at the minor vertices of the second cell 610. Each second axial post 618 can extend from a respective inflow apex 612, through an inflow apex 630 of the second cell 610 and into the opening formed by the second cells 610. The first and second posts 616, 618 can be axially aligned within one another to form a pair of first and second axial posts.

Each pair of axial posts 616, 618 can be configured to receive a respective actuator member to radially expand and/or compress the frame 600, as previously described above for frames 102, 302, 400, and 500. For instance, each axial post 616, 618 can comprise an inner bore within (not shown), and/or house a nut with an inner bore, through which an actuator member (for example, rod 158 or threaded rod 382) can extend. Each bore extending through its respective post 616, 618 and/or nut can be configured to engage the actuator member such that rotation of the actuator member causes the second post 618 to move axially relative to the first post 616. In particular, rotation of the actuator member in a first direction (for example, clockwise) causes corresponding axial movement of the first and second axial posts 616, 618 toward one another, expanding the frame 600. In a similar manner, rotation of the actuator member in a second direction (for example, counterclockwise) causes corresponding axial movement of the first and second axial posts 616, 618 away from one another to compress the frame 600.

In alternative examples, the actuator members can be configured to be pulled or slid axially (rather than rotated) to move the second posts 618 toward the first posts 616 to radially expand the frame. In such examples, the actuator members can be rods, tethers, sutures, cables, wires, and the like.

FIGS. 19 and 20 show that an inflow end portion 632 of one or more support posts 620 can include a cantilevered strut or axial extension 626 disposed between adjacent lower struts 606 and inflow apices 612. Each axial extension 626 can include a fixed end 634 coupled to a respective support post 620, a free end 636 that extends toward the inflow end 602 of the frame 600, and an aperture 638 extending radially through the thickness of the free ends 636. As best illustrated in FIG. 19, the outer most inflow edge of the free ends 636 can be a rounded edge which then extends and tapers toward the fixed ends 634. Accordingly, and as with axial extensions 532 and axial extensions 540, the fixed ends 634 of axial extensions 626 can be said to have a circumferential width that is relatively narrower or lesser than a circumferential width of the free ends 636 of the axial extensions.

The axial extensions 626 can be functionally similar to the axial extensions 344, axial extensions 438, axial extensions 532, and axial extensions 540 described herein. As an example, during the working cycle of the prosthetic valve, the axial extensions 626 can be configured to deflect radially inwardly toward a longitudinal axis of the frame 600 and deflect radially outwardly away from the longitudinal axis and toward an outer boundary of frame 600 (for example, axial extensions 344 and axial extensions 438). In addition to or in lieu of being configured to deflect radially inwardly and outwardly, the axial extensions 626 can be configured to deflect laterally or in a circumferential direction, such as when axial forces act on the free ends 636 (for example, like axial extensions 532 and/or axial extensions 540).

Still referring to FIGS. 19 and 20, each first cell 610 can be formed by two upper struts 640a, 640b and two lower struts 642a, 642b. Each upper strut 640 is coupled on one end to a first axial post 616 and on the other end to a support post 620, while each lower strut 642 is coupled on one end to a second axial post 618 and on the other end the support post 620. The upper struts 640a, 640b can be part of an upper row of struts that defines the outflow end 604 of the frame 600, and the lower struts 642a, 642b can be part of a lower row of struts that defines the inflow end 602 of the frame 600.

One or more lower struts 642 can be selected to have an opening or aperture 646 extending radially through a midsection 648 of the struts. For example, pairs of selected struts 644a, 644b can be arranged along the lower row of struts that define the inflow end 602 of the frame 600. Each pair of selected struts 644 can be a pair of adjacent lower struts 642 that form a pair of adjacent first cells 608. Each selected strut 644, for instance, is coupled on one end to a respective inflow apex 612 and on the other end at a junction of the selected strut and a support post 620. In this way, the ends of the selected struts 644 coupled to an inflow apex 612 of the frame 600 can be referred to as an inflow section 650 and the portions of the selected struts 644 coupled to the support posts 620 can be referred to as an outflow section 652 of the selected struts (FIG. 20). By way of being coupled at one end to an inflow apex 612 and on the other end to support post 620, the inflow and outflow sections 650, 652 of the selected struts 644 can also be said to be configured to bend relative to the midsection 648 as the frame 600 is radially expanded and/or radially compressed.

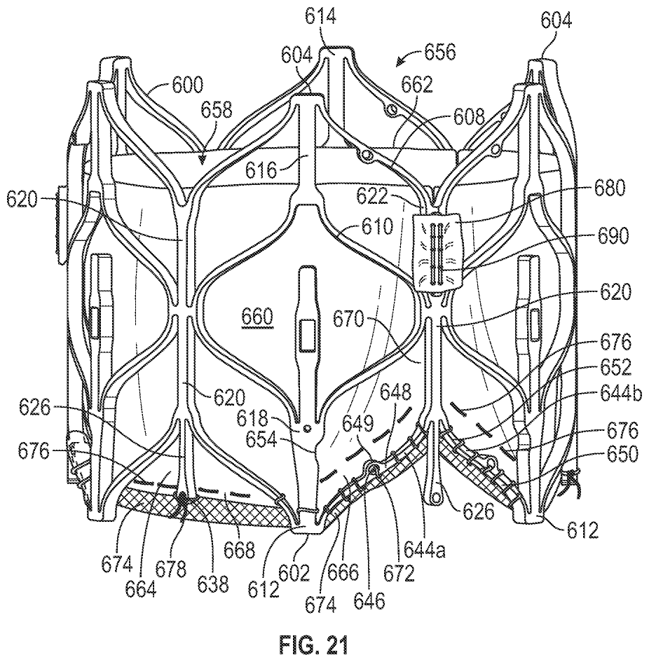

The row of lower struts 642 can also include struts lacking an aperture 646 disposed between each pair of adjacent selected struts 644. The frame 600, for instance, can include a pair of adjacent lower struts 642 lacking an aperture disposed between each pair of adjacent selected struts 644. In this case, the frame 600 can have three pairs of selected struts 644 and three pairs of lower struts 642 lacking an aperture arranged in an alternating fashion circumferentially around the frame 600. The three pairs of alternating selected struts 644, for instance, can be used to couple inflow edge portions of leaflets of valvular structure (for example, FIG. 21). In some examples, each selected strut 644 need not be one of a pair of adjacent selected struts but can be a single selected strut with one or more lower struts lacking an aperture disposed between each successive selected strut 644. In other examples, the selected struts 644 can be grouped in three or more successive selected struts.

In some examples, rather than the selected struts 644 being formed of the lower struts forming adjacent first cells 608, the pair of selected struts can be formed of adjacent lower struts forming the inflow apex 612 of a single first cell 608. For instance, the lower struts 642a, 642b of one or more first cells 608 can each have an aperture extending radially therethrough. Moreover, despite the selected struts being described as having a single aperture within the midsection of the selected struts, it should be understood that the selected struts 644 can have two or more apertures along the inflow, midsection, and/or outflow sections of the struts.

As shown in FIGS. 19 and 20, each of the midsections 648 of the selected struts 644 can form a bump or protrusion 649 at the location of an aperture 646, wherein the protrusion 649 extends into the opening of a first cell 608 and frames a respective aperture 646 extending through the midsection 648. The protrusion 649 can be rounded as shown. As an example and as shown in FIG. 20, when the frame 600 is an expanded state, the protrusion 649 protrudes outwardly relative to the inflow and outflow sections 650, 652 and toward a respective second cell 610 and/or second axial post 618. Hence, to accommodate within the body of the selected strut 644 an aperture 646, the protrusion 649 can have a width greater than a width of the inflow and outflow sections 650, 652 of the selected struts 644.

As best illustrated in FIG. 20, each second axial post 618 extending from an inflow apex 612 and coupled to a respective adjacent selected strut 644 can comprise an indentation 654 configured to receive a protrusion 649 of the strut 644. Specifically, each indentation 654 can be positioned and formed within a longitudinal edge of the second axial post 618 adjacent to a selected strut 644 to receive a protrusion 649 when the frame 600 is in a radially compressed state. In some examples, the indentations 654 can have the same or similar curvature as the inner edges of the protrusions 649 such that the protrusions can be nestled within the indentations 654 when the frame is in a radially compressed state. In forming the indentations 654 to receive the protrusions, for instance, the frame 600 can be compressed to a greater degree than otherwise would be possible in the absence of the indentations 654, as contact between the midsection 648 and the second axial post 618 would prevent a frame without indentations from compressing to the same degree as the frame 600 with indentations 654. In some examples, each indentation 654 receives the entirety of a protrusion, while in other examples, each indentation 654 only receives a portion of a protrusion. For instance, the protrusions 649 need not be entirely received within the indentations 654, but only enough to prevent contact between the selected struts 644 and the second axial posts 618.

In some examples, each second axial post 618 can also be configured to receive protrusions of a respective pair of adjacent struts 644. For example, when a pair of adjacent selected struts 644 form a respective first cell 608, the axial post 618 disposed between the selected struts can have one indentation 654 within one longitudinal edge of the post 618 and another indentation 654 within the opposing longitudinal edge on the other side of the post 618. Accordingly, the indentations 654 on both sides of the posts 618 are configured to receive a respective protrusion when the frame 600 is in a radially compressed state. Additionally, each longitudinal edge of the second axial posts 618 can comprise one or more indentations 654, such as when an adjacent selected strut 644 has two or more protrusions 649 forming apertures 646 along the midsections 648 and inflow and/or outflow sections 650, 652.

FIG. 21 illustrates an example of a prosthetic valve 656 in a radially expanded configuration and comprising the frame 600. The prosthetic valve 656 can comprise similar features and functionality as prosthetic valve 100 and prosthetic valve 300, as previously described.

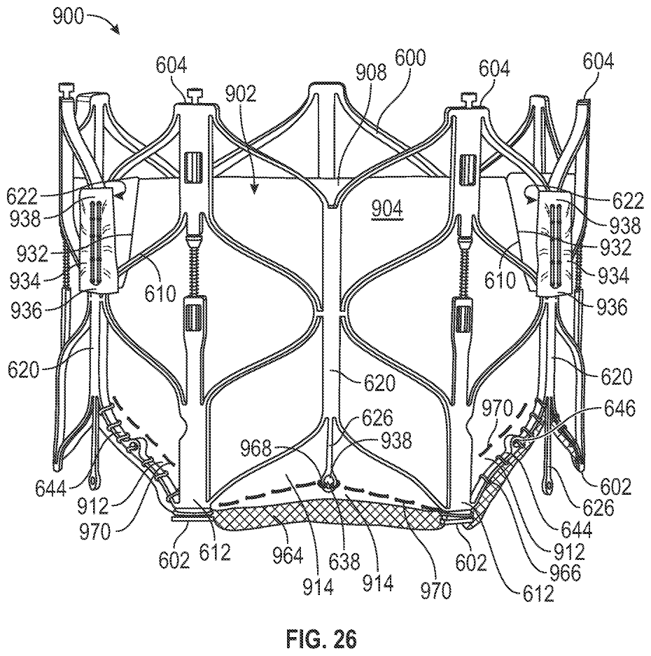

The prosthetic valve 656 includes a valvular structure 658 which is coupled to and supported inside the frame 600 and configured to regulate the flow of blood through the prosthetic valve 656 from the inflow end 602 to the outflow end 604. The valvular structure 658 can include a leaflet assembly comprising one or more leaflets 660 made of flexible material and having the same structural features and shape as the leaflets 116, leaflets 354, or leaflets 904 (FIGS. 26-28C) described herein. The leaflets 660 can, for instance, be made from in whole or part, biological material, bio-compatible synthetic materials, or other such materials. Suitable biological material can include, for instance, bovine pericardium (or pericardium from other sources).

Each leaflet 660 of the valvular structure 658 can include a main body, an outflow edge portion 662, and the inflow edge portion 664. The inflow edge portion 664 of each leaflet 660 can comprise angled edge portions 666 and an apex edge portion 668. Each leaflet can also include one or more tabs located on opposite sides of the main body and outflow edge portion 662 of the leaflet 660 (for example, tabs 172, 174 in FIG. 4). Each leaflet can have axially extending side edge portions 670 extending between a tab and an angled edge portion 666. Each leaflet 660 of the valvular structure 658 can be secured to one another at their adjacent tabs to form respective leaflet commissures 680, each of which can be secured to a commissure support member 622 (for example, radially through window 624) via sutures 690.

As illustrated in FIG. 21, the valvular structure 658 can include three leaflets 660 mounted within the frame 600 and arranged to collapse in a tricuspid arrangement. The inflow edge portions 664 of the leaflets 660 can generally define a scalloped shaped edge that tracks the row of lower struts 642 around the frame 600. Each inflow edge portion 664 can be coupled to one or more selected struts 644 and/or axial extensions 626 having respective apertures 646, 638.

The angled edge portions 666, for instance, can be coupled to selected struts 644 forming first cells 610 and inflow apices 612, and which extend between the inflow apices 612 and the support posts 620. Specifically, as shown in FIG. 21, adjacent angled edge portions 666 of adjacent leaflets 660 can be coupled to a respective pair of adjacent selected struts 644a, 644b. In this arrangement, the angled edge portions 666 of the leaflets 660 largely track pairs of selected struts 644 circumferentially around frame 600. In some examples, such as the example illustrated in FIG. 21, the valve 656 can have a pair of adjacent lower struts 642 lacking an aperture spanning the circumferential gap between pairs of selected struts 644 and adjacent pairs of inflow apices 612 which have the apex edge portions 668 of the leaflets 660 extending therebetween. In such examples, the scalloped shaped edge of the leaflets 660 can be said to track the selected struts 644 circumferentially around the frame 600. In alternative examples, however, one or both lower struts 642 disposed between pairs of selected struts 644 can also have an aperture 646 and thereby be configured as a selected strut, such as to couple one or more soft components of the prosthetic valve 656 to the frame 600.

Sutured to the inflow edge portions 664 of the leaflets 660 can also be a fabric connecting skirt 674 used to connect each angled edge portion 666 (and apex and axial portions) of the leaflets 660 to a corresponding selected strut 644a or 644b. The connecting skirt 674 can, for instance, be coupled to the inflow edge portions 664 via sutures 676 and resist tearing that the leaflets 660 alone may be unable to resist during operation of the valve 656 if the leaflets were sutured directly to the frame 600. As depicted in FIG. 21, sutures 672 are used to couple the inflow edge portions of the leaflets to the frame. The sutures 672 can form whip stitches that extend through the apertures 646 within the midsection 648 of the selected struts 644 and through the connecting skirt 674 (and optionally the leaflets) to secure the leaflets 660 to the frame 600. Sutures 672 can also extend around the remaining portions of the struts 644, such as around the inflow and outflow sections 650, 652, and through the connecting skirt 674 (and optionally through the leaflets).

As illustrated in FIG. 21, the ends of the angled edge portions 666 nearest the apex edge portion 668, can be coupled to the lower ends of the lower struts 642, selected struts 644, and/or to the posts 618 that extend between the major and minor vertices of respective first and second cells 608, 610. As such, the apex edge portions 668 are positioned at or substantially at the inflow end 602 of the frame 600. The axial edge portions 670 of the leaflets 660 can extend along the support posts 620 disposed between adjacent first cells 608. In some examples, the axial edge portions 670 are also coupled to the support posts 620.

The apex edge portions 668 of the leaflets 660 can extend between adjacent inflow apices 612 and be secured or anchored to the frame 600 via an axial extension 626. In particular, the apex edge portion 668 of each leaflet extends between respective inflow apices 612 and is directly coupled to the frame 600 only at a corresponding axial extension 626 via sutures 678 extending through aperture 638 of the free end 636 of the axial extension. As such, in the illustrated example, those portions of the apex edge portion 668 on either side of the axial extension 626 are unanchored. The outer ends of the apex edge portion 668 near the angled edge portions 666 can be coupled to the posts 618 and/or lower struts 642, 644 that form the inflow apices 612 such that a substantial portion of the apex edge portion 668 is still left unanchored. Accordingly, the inflow edge portions 664 of the leaflets 660, and more specifically, the apex edge portions 668 can be configured to move with the axial extensions 626 radially inwardly and outwardly relative a longitudinal axis of the frame 600 and/or laterally toward one or both respective inflow apices 612, depending on the flexibility of the axial extensions, as described above.

The prosthetic heart valve 656 can also include an outer skirt (not shown) mounted to the outer surface of the frame 600 (for example, outer skirt 374 and outer skirt 548), as previously described. The outer skirt can extend circumferentially around the outer surface of the frame 600 and extend axially from an inflow end portion of the outer skirt to an outflow end portion of the skirt. The outer skirt of the valve 656 can, for instance, be connected to respective lower struts of the first and second cells 608, 610. As an example, the outer skirt can be sutured to the selected struts 644 coupled to the leaflets 660 such that the sutures 672 extending through the apertures 646 also extend through the outer skirt. In such an example, the sutures 672 can also extend through the connecting skirt 674 sutured to the inflow edge portions 664 of the leaflets 660. In addition to or in lieu of connecting the outer skirt to selected struts 644, the outer skirt can be connected to the lower struts which form the second cells 610 coupled to the second axial posts 618. In either case, the outer skirt can be connected to the lower struts of the second cells 610 and/or the selected struts 644 (for example, inflow and outflow sections 650, 652) via whip stitching extending around the struts and extending through the outer skirt.

In some examples, the outer skirt can also be coupled to the inflow edge portion 664 of one or more leaflets 660 using whip stitching that extends through the apex edge portions 668 and inflow end of the skirt and around the apex edge portions and the inflow end of the skirt. This allows the inflow end portion of the skirt to move laterally and/or radially inwardly and outwardly with the axial extensions 626 and apex edge portions 668 of the leaflets during working cycles of the prosthetic valve 656. In further examples, the inflow end portion of the outer skirt can itself be coupled to the axial extensions 626 via stitching extending around the axial extensions 626 and/or through the apertures 638. In alternative examples, one or more other fasteners, such as pins or screws, can be used to secure the outer skirt to the axial extensions 626.

As shown in FIGS. 19-21, the frame 600 comprises six first cells 608 that can extend circumferentially in a row, with a second cell 610 within each first cell. The frame can also include six pairs of posts 616, 618 coupled to respective pair of cells 608, 610, six support posts 620, six axial extensions 626, six selected struts 644, and three commissure support members 622. In other examples, however, the frame 600 can comprise a greater or fewer number each of these features listed.

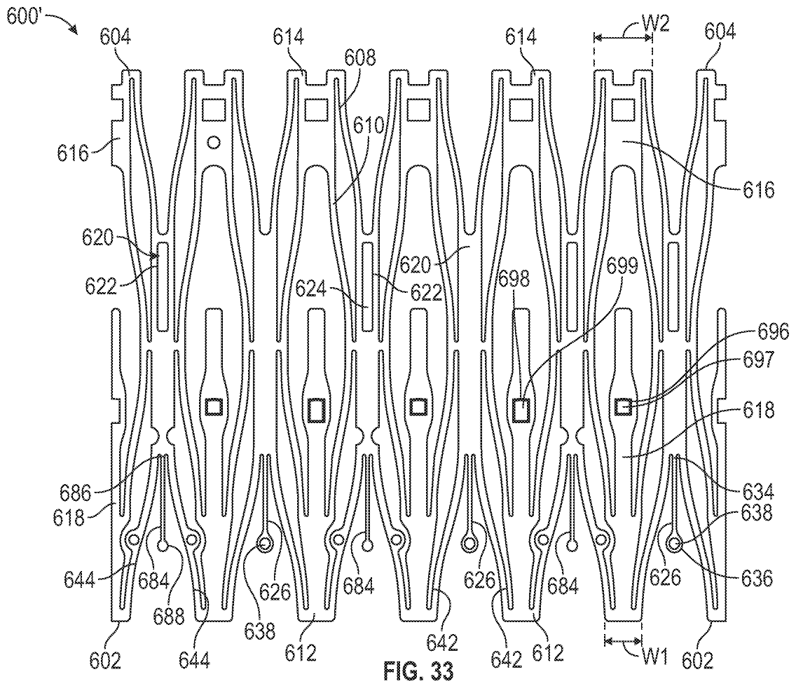

FIG. 33 illustrates a frame 600′ according to another example. The frame 600′ is shown in a compressed and flattened state and can include all of the components as previously described in connection with frame 600 of FIGS. 19-21 and 36. For the sake of brevity, a detailed description of the frame 600 is not repeated here.

One difference between the frame 600 and frame 600′ is the relative widths of the inflow apices 612 and the outflow apices 614. Specifically, each inflow apex 612 of the frame 600′ can have a circumferential width W1 that is relatively narrow to or less than a circumferential width W2 of each outflow apex 614. For instance, each outflow apex 614 can generally be configured to have a relatively wide or greater circumferential width W2 than each inflow apex 612 to adequately support an inner bore and/or a nut with an inner bore, through which an actuator member can extend (for example, rod 158 or threaded rod 382). Each actuator member in this instance, can extend into a respective pair of first and second axial posts 616, 618 configured to receive the actuator member so far as to avoid having the actuator member extend into or through a respective inflow apex 612. For instance, the axial posts 616, 618 can be configured to radially expand and/or compress the frame 600′ as previously described above for frames 102, 302, 400, 500, and 600. Therefore, unlike the outflow apices 614, each inflow apex 612 need not have a circumferential width to support an actuator member and can have a circumferential width W1 comparatively narrow to or less than the circumferential width W2 of the outflow apices 614. Since a relatively larger portion of the soft components of a prosthetic valve can be located in the inflow region of the valve, the relatively narrow width W1 of the inflow apices 612 advantageously provides greater space for the soft components to be folded around the inflow end 602 of the frame 600′, which reduces the overall compressed or crimped profile of the valve during delivery.

Another difference between frame 600 and frame 600′ is that two or more sets of selected second axial posts 618 (or axial post 617) of the frame 600′ can include respective window and nut pairings which are dissimilarly dimensioned from one another. Each selected second axial post 618, for instance, can define or frame a fully enclosed window 696, 698 that houses a respective nut 697, 699 for receiving an actuator member. A first set of windows 696 of a first set of second posts 618 can house a first set of nuts 697 and a second set of windows 698 of a second set of second posts 618 can house a second set of nuts 699. As shown in FIG. 33, the first window and nut pairings 696, 697 and the second window and nut pairings 698, 699 can have different axial lengths relative to one another. In particular, the first window and nut pairings 696, 697 can be relatively truncated and shorter in length in comparison to the second window and nut pairings 698. The first window and nut pairings 696, 697 can generally be square in shape, while the second window and nut pairings 698, 699 can be rectangular in shape and extend lengthwise in the direction of the inflow and outflow ends 602, 604 of the frame 600′. The second window and nut pairings 698, 699 by extension can be comparatively elongate and longer in length in comparison to the first window and nut pairings 696, 697. Additionally or alternatively, a circumferential width of the first window and nut pairings 696, 697, such as in the circumferential direction of the frame 600′, can be relatively greater or lesser than a circumferential width of the second window and nut pairings 698, 699 to achieve similar differentiation. Other differences in dimensions and shapes can also be implemented as desired.

Each nut 697, 699 can be visible through its respective window 696, 698 of the second post 618 and include an inner bore configured to engage an actuator member (for example, the rod 158 or the threaded rod 382). The frame 600′ can be radially expanded and/or compressed with relative movement of the actuator member as described herein. In the illustrated example of FIG. 33, the frame 600′ includes a first set of selected second posts 618 having three first window and nut pairings 696, 697 and a second set of selected second posts 618 having three second and window pairings 698, 699, each combination configured to receive and engage a respective actuator member. In some examples, one or more of these actuator members can be configured as “right-handed” actuators in which rotation of the actuator members in a clockwise direction effects expansion of the frame 600′. Alternatively, the actuator members can be configured as “left-handed” actuators in which rotation of the actuator members in a counterclockwise direction also effects expansion of the frame 600′. Rotation of the right-handed actuator members in a counterclockwise direction and left-handed actuator members in a clockwise direction can similarly effect compression of the frame 600′.

In some examples, the first window and nut pairings 696, 697 can be exclusively associated with either the right- or left-handed actuator members, while the second window and nut pairings 698, 699 can be exclusively associated with the other of the right- or left-handed actuator members not associated with the first window and nut pairings 696, 697. As one example, right-handed actuator members can be exclusively associated with and engage only the first window and nut pairings 696, 697 and left-handed actuator members can be exclusively associated with and engage only the second window and nut pairings 698, 699. In this way, the visible dissimilarities between the first window and nut pairings 696, 697 and second window and nut pairings 698, 699 can identify which type of actuator member extends through each (for example, via the differences in lengths, widths, and/or shapes). As in the above example, for instance, the right-handed actuator members can be identified by way of the respective first window and nut pairings 696, 697 and the left-handed actuator members can be identified by the second window and nut pairings 698, 699. The identification between the right- and left-handed actuator members can be helpful during assembly of a prosthetic valve and/or during implantation of the valve when radially expanding or compressing the frame. When used during implantation, for example, either one or both nuts 697, 699 can be made of a radiopaque material that has a different radiopacity than a radiopaque material used to form their respective second posts 618. As such, one or both types of nuts 697, 699 can have a radiodensity different than the radiodensity of the second posts 618 which can allow for the identification between the actuator members via imaging during implantation procedures.

As illustrated in FIG. 33, another difference between the frame 600 and frame 600′ is t the structure of one or more axial extensions. In particular, the frame 600′ can include one or more cantilevered struts or axial extensions 684, which lack an aperture, disposed between adjacent lower struts 644 (and/or lower struts 642) and inflow apices 612. Like the axial extensions 626, each axial extension 684 can include a fixed end 686 coupled to a respective support post 620 and a free end 688 that extends toward the inflow end 602 of the frame 600′. Unlike the axial extensions 626, however, the axial extensions 684 lack an aperture (for example, aperture 638) extending radially through the thickness of the free ends 688. As indicated herein, the apertures 638 of the axial extensions 626 are generally sized and shaped to allow a suture and/or a head of needle to pass therethrough for suturing the leaflets and/or other soft components to the frame 600′. This leads to the free ends 636 of the axial extensions 626 to be relatively wider or greater in diameter, which can increase the compressed or crimped profile of the valve. Accordingly, due to the lack of an aperture, the free ends 688 of the axial extensions 684 can be relatively narrow or smaller in diameter in comparison to the rounded free ends 636 of the axial extensions 626. As with the inflow apices 612, the relatively narrow profile of the axial extensions 684 can provide relatively greater space over that space provided by the axial extensions 626, which can reduce the overall compressed profile of the valve during delivery.

With the absence of an aperture, in some instances, the axial extensions 684 can be positioned along the frame 600′ where an axial extension is not directly coupled to a leaflet. As an example, FIG. 33 shows that each axial extension 684 can extend axially from a respective support post 620 comprising a commissure support member 622 and midway between pairs of respective adjacent inflow apices 612 and selected struts 644. Each axial extension 684 in this respect can be aligned with a leaflet commissure formed of adjacent leaflets disposed inside the frame 600′ (for example, commissures 680) and can extend between adjacent inflow apices 612 which lack a leaflet extending therebetween (for example, similarly situated to the front, right axial extension 626 shown in FIG. 21). For instance, a scalloped shaped edge of the leaflets, generally formed of the inflow edges of the leaflets (for example, inflow edge portions 664), can be coupled to and track the selected struts 644 and axial extensions 626 circumferentially around the frame 600′, such that the leaflets occupy relatively little, if any, space between the pairs of adjacent inflow apices 612 and selected struts which have an axial extension 684 disposed in between. The axial extensions 684 lacking an aperture are therefore, left unattached to the leaflets. Although not coupled to a leaflet, the axial extensions 684 can be configured and used to prevent soft components, such as an outer skirt mounted to the outer surface of the frame 600′, from extending radially inwardly into the frame 600′, while also reducing the overall compressed profile of the valve. In such an example, the axial extensions 684 can be configured to have a degree of rigidity suitable to prevent or limit movement of the outer skirt radially inwardly into the frame 600′. In further examples, the axial extensions 684 can also be configured to provide a degree of deflection radially inwardly toward a longitudinal axis of the frame 600′ and/or laterally toward respective inflow apices 612.

As illustrated in FIG. 33, the frame 600′ can include three axial extensions 626 with an aperture 638 and three axial extensions 684 without an aperture. The axial extensions 626 and axial extensions 684 can be arranged circumferentially around the frame 600′ in an alternating pattern such that each axial extension 684 is located between a respective pair of axial extensions 626. Each axial extension 626, as such, can be located between a respective pair of axial extensions 684. In some examples, the axial extensions 626 and axial extensions 684 can be arranged in various configurations and/or be included in any greater or fewer numbers relative to one another.

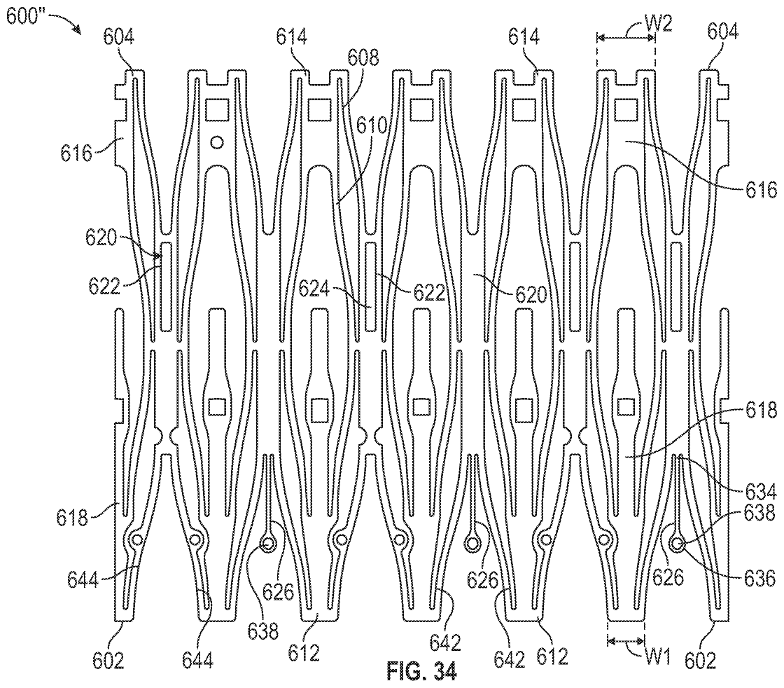

FIG. 34 illustrates a frame 600″ according to another example in a compressed and flattened state and can, like frame 600′, include all of the components as previously described in connection with FIGS. 19-21. Unless stated otherwise, the frame 600″ can also include all of the components as described in connection with frame 600′ and FIG. 33. As with frame 600′, a detailed description of frame 600 is not repeated here.

In comparison to frame 600′, and as shown in FIG. 34, the frame 600″ only includes axial extensions 626 disposed between every other pair of adjacent of inflow apices 612. Specifically, one difference between the frame 600′ and frame 600″, is that rather than including and arranging axial extensions 626 and axial extensions 684 in an alternating fashion circumferentially around the frame, each axial extension 684 without an aperture is removed such that the only axial extensions of the frame 600″ are the axial extensions 626 disposed between their respective pairs of adjacent inflow apices 612. As illustrated in FIG. 34, the axial extensions 626 of frame 600″ extend axially between adjacent lower struts 642, while the circumferential between each pair of adjacent selected struts 644 and respective inflow apices 612 lacks an axial extension. Yet, in some examples, each axial extension 626 can extend axially between adjacent selected struts 644, while the circumferential gap between each pair of adjacent lower struts 642 and respective inflow apices 612 lack an axial extension. Consequently, the frame 600″ includes a lesser number of axial extensions than either frame 600 or frame 600′, and thereby provides relatively greater space at the inflow region of the frame 600″. This, alone or in conjunction with the features described herein in connection with the frame 600 and frame 600′, can reduce the overall crimped profile of a compressed valve comprising the frame 600″.

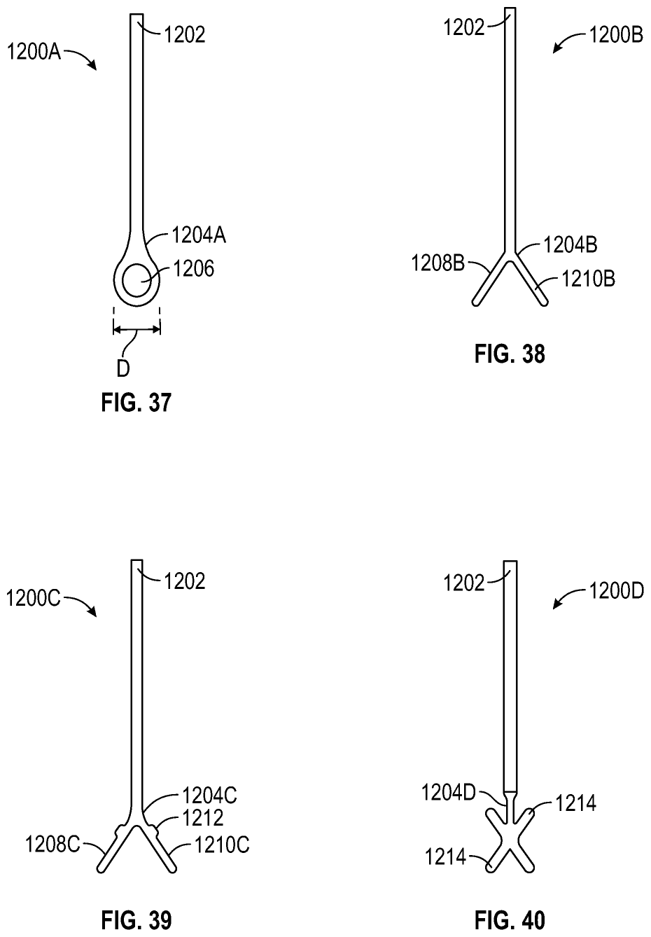

FIGS. 37-44 show various examples of cantilevered axial extensions 1200A-1200H which can be implemented with any of the frames described herein in lieu of or in combination with any of axial extensions described herein (for example, the axial extensions 344, 438, 532, 540, 626, or 810). Each axial extension 1200A-1200H can generally include a fixed end 1202 coupled to a respective frame and a free end 1204A-1204H that extends toward the inflow end of the frame. As illustrated in FIGS. 37-44, the free ends 1204A-1204H of the axial extensions 1200A-1200H can have structural differences, which can permit a frame with the axial extensions to achieve a relatively minimal compressed profile. The fixed end 1202 can be connected to a frame, such as to a respective support post, as previously described.

FIG. 37 depicts a cantilevered axial extension 1200A generally similar in structure to the axial extensions 344, axial extensions 438, axial extensions 532, axial extensions 540, and axial extensions 626 described herein. That is, the free end 1204A of the axial extension 1200A can include an aperture 1206 extending radially through the thickness of the extension. The aperture 1206 can be sized and shaped to receive one or more sutures, other fasteners, and/or soft components of a valve. One result of having the aperture 1206, is that the free end 1204A of the axial extension 1200A has a circumferential width or diameter D to accommodate a needle used to thread suture through the aperture. In some instances, the degree of space that the diameter D of the free end 1204A occupies when a frame having the axial extensions 1200A is radially compressed can limit the overall crimped profile of the frame and valve. Such limited compression or crimped profile of the frame may occur when the outer edges of the free ends 1204A abut against a pair of adjacent lower struts of the frame (for example, lower struts 370, lower struts 642, etc.).

Alternatively, the free ends 1204B-1204H of the following axial extensions 1200B-1200H can be structured in such a way as to compress when contacted by the frame struts. That is, the free ends 1204B-1204H of the axial extensions 1200B-1200H can be compressible as the frame is radially compressed such that the free ends 1204B-1204H achieve a relatively smaller circumferential profile than the diameter D of the free ends 1204A of the axial extensions 1200A and thereby, occupy less space. This compressibility of the free ends 1204B-1204H can permit a frame having one or more of the axial extensions 1200B-1200H to compress to a relatively greater extent than a frame having the axial extensions 1200A.

FIGS. 38 and 39 show two different, but structurally similar examples of compressible cantilevered axial extensions 1200B, 1200C. In particular, the axial extensions 1200B, 1200C can include respective free ends 1204B, 1204C that are V-shaped. Each free end 1204B, 1204C, for instance, can have a respective pair of adjacent arms 1208, 1210 which extend outwardly from a common junction in a V-like shape and in circumferential directions of a respective frame. The arms 1208B, 1210B of the axial extension 1200B and the arms 1208C, 1210C of the axial extension 1200C can be constructed to deflect laterally toward one another about their common junction such that when the arms 1208, 1210 are in a deflected state, the axial extensions 1200B, 1200C have a relatively narrower circumferential profile than the uncompressed profiles depicted in FIGS. 38 and 39. In the deflected state, the circumferential profiles of the axial extensions 1200B, 1200C are less than the diameter D of extension 1200A. Such deflection can occur when the arms 1208, 1210 contact adjacent struts of a frame during radial compression. To secure respective leaflets or other soft components to the axial extensions 1200B, 1200C, sutures can form whip stitching extending through and around the edge portions of the leaflets or soft components, and around the arms 1208, 1210 of the axial extensions 1200B, 1200C. The sutures can also wrap around the junction of respective arms 1208, 1210.

One difference between the axial extension 1200B and axial extension 1200C is that the extension 1200C includes one or more bumps or projections 1212 along longitudinal edges of the arms 1208C, 1210C. These projections 1212 can prevent or reduce the likelihood sutures extending around and above the projections move or slide along and off the arms 1208C, 1210C.

FIG. 40 illustrates a compressible cantilevered axial extension 1200D according to another example. The axial extension 1200D can include a free end 1204D having a plurality of outwardly extending arms 1214 arranged in an X-like shape and in circumferential alignment with the frame. In a similar fashion as the arms 1208, 1210 of the axial extensions 1200B, 1200C, adjacent pairs of the arms 1214 can deflect laterally toward one another such that the free end 1204D can have relatively narrower and compressed profile than the uncompressed or expanded profile shown in FIG. 40. For example, the two upper most arms 1214 and the two lower most arms 1214 shown in FIG. 40 can deflect toward one another, respectively. A suture or other fastener can extend in and out of the plane of FIG. 40 and around each arm 1214 as well as between adjacent pairs of arms 1214 and/or around the common junction of the arms 1214, such as in a crisscross fashion.

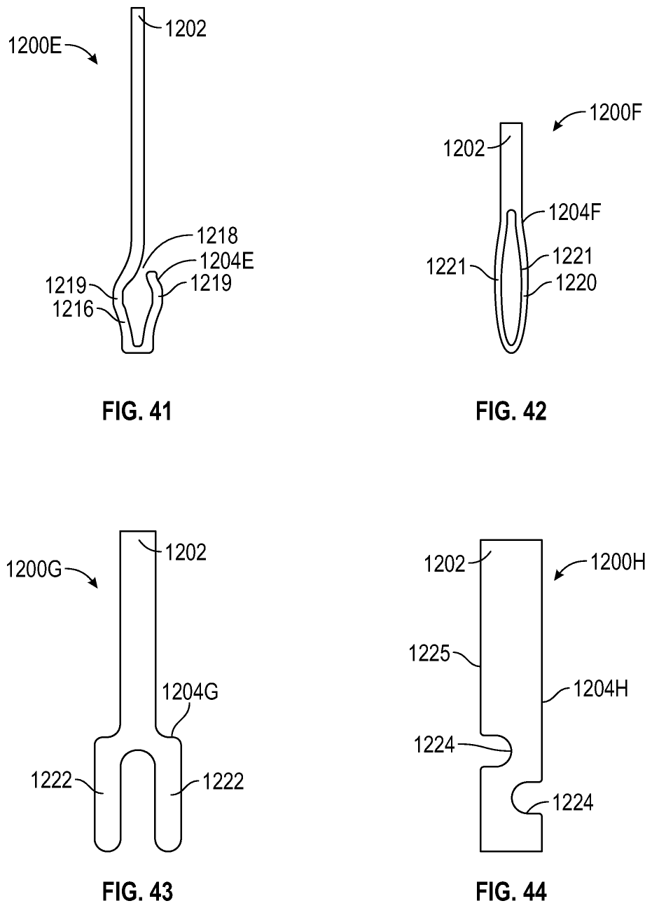

Two further examples of compressible cantilevered axial extensions 1200E, 1200F are illustrated in FIGS. 41 and 42. The axial extensions 1200E, 1200F can include respective free ends 1204E, 1204F that define a compressible eyelet. The free end 1204E of the axial extension 1200E, for instance, can include a U-shaped eyelet 1216 having a discontinuous or open segment 1218. Sutures can extend through the opening and around the framing of the eyelet 1216 and/or slide through the open segment 1218 to secure respective leaflets and/or other soft components to the axial extension 1200E. When a frame including the axial extension 1200E is radial compressed, the side or lateral portions 1219 of the free end 1204E that frame the U-shaped eyelet 1216 can move toward one another and converge, narrowing or closing the open segment 1218.

In a similar manner, the free end 1204F of the axial extension 1200F can frame and define an enclosed elliptical or elongate eyelet 1220. As with the axial extension 1200E, the side or lateral portions 1221 of the free end 1204F framing the eyelet 1220 move toward one another as adjacent portions of a respective frame contact the free end 1204F during radial compression of the frame, resulting in a relatively minimal circumferential profile. To secure respective leaflets or other soft components to the axial extension 1200F, sutures can form whip stitches that extend through and around the edges of the leaflets and/or soft components, through the eyelet 1220, and around the lateral portions 1221.

FIG. 43 depicts another compressible cantilevered extension 1200G having a U-shaped free end 1204G defined by a pair of adjacent arms 1222. The arms 1222 can be circumferentially spaced from one another and define a circumferential space therebetween to receive a suture or other fastener. Each arm 1222 can be configured to deflect toward the other and into the space extending between the arms 1222 such that the free end 1204G of the axial extension 1200G can have circumferential profile relatively narrower than the diameter D of extension 1200A when a respective frame is radially compressed. A suture can extend through and around the leaflets and soft components of a respective valve and between and around the arms 1222. Fasteners can also wrap around a common junction of the arms 1222.

As illustrated in FIG. 44, another example of a compressible cantilevered axial extension 1200G can include a free end 1204H having longitudinal edges 1225 with one or more cutouts 1224 there along. The cutouts 1224 can be located along one or both longitudinal edges 1225 of the extension 1200G. Each cutout 1224 can be sized and shaped to receive a suture or other fastener extending through and around leaflets and/or other soft components of a respective valve. Such fasteners or sutures can also extend across the axial extension 1200H from one cutout 1224 to another, such as in a crisscross fashion.

It should be appreciated that each of the cantilevered extensions 1200A-1200H can also be functionally similar to the axial extensions described herein (for example, the axial extensions 344, axial extensions 438, axial extensions 532, axial extensions 540, and axial extensions 626). As an example, during the working cycle of the prosthetic valve, one or more of the axial extensions 1200A-1200H can be operable to deflect radially inwardly toward a longitudinal axis of a respective frame and deflect radially outwardly away from the longitudinal axis and toward an outer boundary of frame (for example, axial extensions 344 and axial extensions 438). In addition to or in lieu of being configured to deflect radially inwardly and outwardly, the axial extensions 1200A-1200H can be configured to deflect laterally or in a circumferential direction, such as when axial forces act on the free ends 1204A-1204H (for example, like axial extensions 532 and/or axial extensions 540).

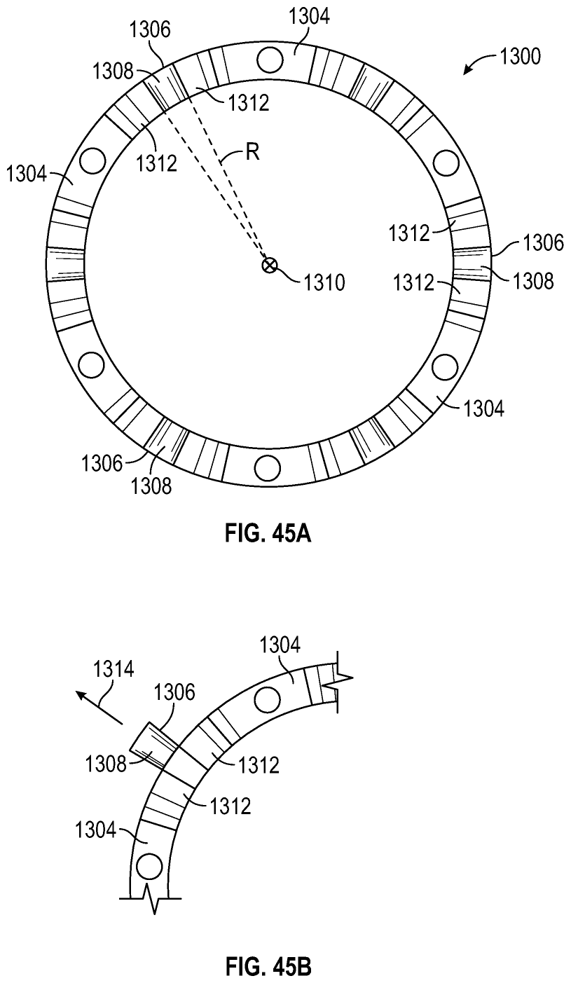

Any of the cantilevered extensions described herein can also be fashioned to taper radially inwardly toward the center of a respective frame, including axial extensions 344, axial extensions 438, axial extensions 532, axial extensions 540, axial extensions 626, and axial extensions 1200A-1200H. As one example, FIGS. 45A and 45B depict an inflow end 1302 of a frame 1300 according to one implementation. FIGS. 45A and 45B show the frame 1300 in a radially expanded state and a radially compressed state, respectively. The frame 1300, which is shown from an “upward” view along a central longitudinal axis 1310 of the frame in FIG. 45A, can include a plurality of inflow apices 1304 circumferentially spaced apart from one another along a circumference of the frame 1300. Between each pair of adjacent inflow apices 1304 is a respective axial extension 1306 having a respective free end 1308.

As indicated by the radial lines R in FIG. 45A, one or more of the free ends 1308 of the axial extensions 1306 can taper radially inwardly toward the longitudinal axis 1310 of the frame 1300. For purposes of illustration, only the free ends 1308 of the axial extensions 1306 are shown. Each axial extension 1306 can also be situated between a respective pair of lower struts 1312. Each lower strut 1312 extends between a respective inflow apex 1304 and a common junction of the pair of struts 1312. In some examples, each axial extension 1306 extends from the common junction of the struts 1312.

As shown in FIG. 45B, which shows an enlarged view of a single axial extension 1306, when the frame 1300 is radially compressed and a pair of adjacent lower struts 1312 move circumferentially toward one another, the pair of lower struts 1312 contact the free end 1308 of the axial extension 1306. Due to the tapered shaped of the free end 1308, as the lower struts 1312 contact and press the lateral portions of the free end 1308, the free end 1308 moves radially outwardly from the applied pressure. The radially outwardly movement of the axial extension 1306 is generally indicated by arrow 1314. In this way, the tapered axial extensions 1306 can permit the frame 1300 to compress to a relatively greater extent than a frame which lacks the tapered axial extensions 1306. In some examples, the free ends 1308 move only partially radially outwardly under pressure applied by the frame. In other examples, the free ends 1308 move entirely or nearly entirely outside an outer boundary of the frame 1300 (FIG. 45B). It should also be appreciated that, in some examples, the free ends 1308 of the axial extensions 1306 can also be compressible, similarly to the axial extensions 1200B-1200H. In such examples, the free ends 1308 can initially be compressed upon contact with the lower struts 1312 and then move radially outwardly as the pressure increases and the frame further compresses.

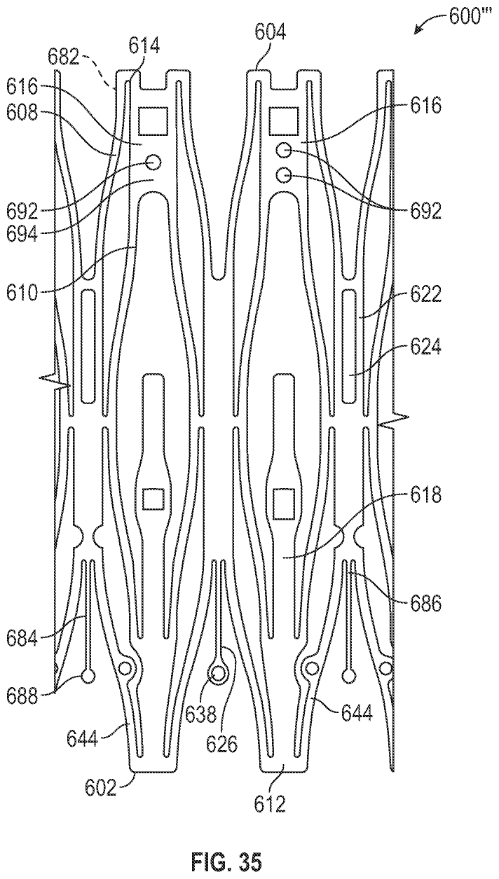

FIG. 35 illustrates a portion of a frame 600′″ according to another example. The frame 600′″ is shown in a radially compressed and flattened state and can include all of the components previously described in connection with frame 600 and FIGS. 19-21, in addition to all of the components described in connection with frame 600′, frame 600″, and respective FIGS. 33 and 34. Although only a portion of the frame 600′″ is illustrated, it should be appreciated that the frame 600′″ forms an annular structure in the same way that frame 600, frame 600′, and frame 600″ each form an annular structure.

As shown in FIG. 35, one or more of the first axial posts 616 can include one or more apertures 692. As previously described, each first axial post 616 can extend from a respective outflow apex 614 at the outflow end 604 of the frame 600′″ and comprise an inner bore (not shown) through which an actuator member can extend (for example, rod 158 or threaded rod 382). Each aperture 692 shown in FIG. 35 can be sized and shaped to extend between an external surface of the frame 600′″ and an inner bore such that each aperture is in communication with the bore of the post 616. In particular, each aperture 692 can extend from an inner surface 682 and/or an outer surface 694 of the frame 600′″, to the inner bore of the first axial post 616. As shown along the surface of the right axial post 616 in FIG. 35, one or more of the posts 616 can include two or more apertures 692 axially spaced from one another. One or more of the second axial posts 618 can also include one or more apertures 692 extending from an external surface of the frame 600′″ to a respective inner bore. Although not shown, one or more of second axial posts 618 can include one or more apertures 692 that are in communication with a respective bore or channel of the post 618. In lieu of apertures 692 in the first posts 616, any of the second posts 618 can include one or more apertures 692.

Any of the examples of frames disclosed herein can include one or more such apertures 692 in one or more axial posts of the frame.

FIG. 36 depicts a section of a frame 600″″ for a prosthetic valve, according to another example. While only a single cell column comprising a first (outer) cell 609 and a second (inner) cell 611 is shown, it should be appreciated that the complete frame 600″″ includes a plurality of cell columns (each comprising a pair of first and second cells 609, 611) and respective first and second axial posts 617, 619 depicted in FIG. 36. The frame 600″″ can include all of the components previously described in connection with frame 600, frame 600′, frame 600″, or frame 600′″, except for the differences described below.

As shown in FIG. 36, the arrangement of first and second axial posts 617, 619 are inverted from the arrangement of the first and second axial posts 616, 618 shown in FIGS. 19-21. That is, the second axial post 619 can extend from a respective outflow apex 614 of the frame 600 to an outflow apex 628 of the second cell and into the second cell 611. Each first axial post 617 can extend from a respective inflow apex 612 of the frame to an inflow apex 630 of the second cell 611. The axial posts 617, 619 can be axially aligned.

In comparison to other frames described herein, which are generally depicted as having actuator members (for example, threaded rods 382) that are configured to be releasably coupled to actuator assemblies (for example, actuator assemblies 1108) of a delivery apparatus at the outflow end of the frame, the frame 600″″ shown in FIG. 36 is configured to be releasably coupled to actuator assemblies of a delivery apparatus at the inflow end of the frame. In particular, the first and second axial posts 617, 619 can include an inner bore (not shown) extending along the length of the first axial post 617 and at least a length of the second post 619. An actuator member 621 such as in the form of a threaded rod can extend through the inner bore of the first and second axial posts 617, 619, and has a head portion 621a disposed adjacent an inflow apex 612 of the frame. The actuator member 621 can be configured to control the radial expansion and compression of the frame 600. The actuator member 621 can engage the bore either through threaded engagement, a push-pull configuration, or a tethering system, such as those mentioned in connection with FIG. 3 and frame 102. In some examples, a nut 623 can be visible through a window 625 framed by the second axial post 619 and include an inner threaded bore configured to engage the actuator member 621 (FIG. 33).

Advantageously, the inverted arrangement of the first and second axial posts 617, 619 can allow the configuration and other features of the frame 600 to remain largely the same but extend the use of the frame 600 to other implantation locations and/or delivery approaches. As one example, a prosthetic valve constructed using the frame 600 and having the inverted first and second axial posts 617, 619 can be advanced transeptally by a delivery apparatus (for example, the delivery apparatus 1100) from the right atrium to the left atrium and toward the native mitral valve of a patient. A prosthetic valve in this case can be positioned within the native mitral valve and disengaged from the delivery apparatus in a way that the inflow end 602 of the frame 600 is located within the left atrium for regulating blood flow from the left atrium into the left ventricle. In another example, by coupling the delivery apparatus to the inflow end 602 of the frame 600″″, the prosthetic valve can be delivered to the native aortic valve via a transapical delivery approach wherein the prosthetic valve and the delivery apparatus is inserted into the left ventricle via a surgical opening in the apex of the left ventricle. For comparison, the example of the frame 600 shown in FIGS. 19-21 and having the first and second axial posts 616, 618, can receive actuator members at the outflow end 604. This example of the frame 600 can be desirable, for instance, for implantation into the aortic valve, such as when the prosthetic valve is delivered through the native aortic arch. Any of the prosthetic valve frames disclosed herein can be adapted to include actuator members 621 that have head portions 621a at the inflow end of the frame rather than at the outflow end of the frame.

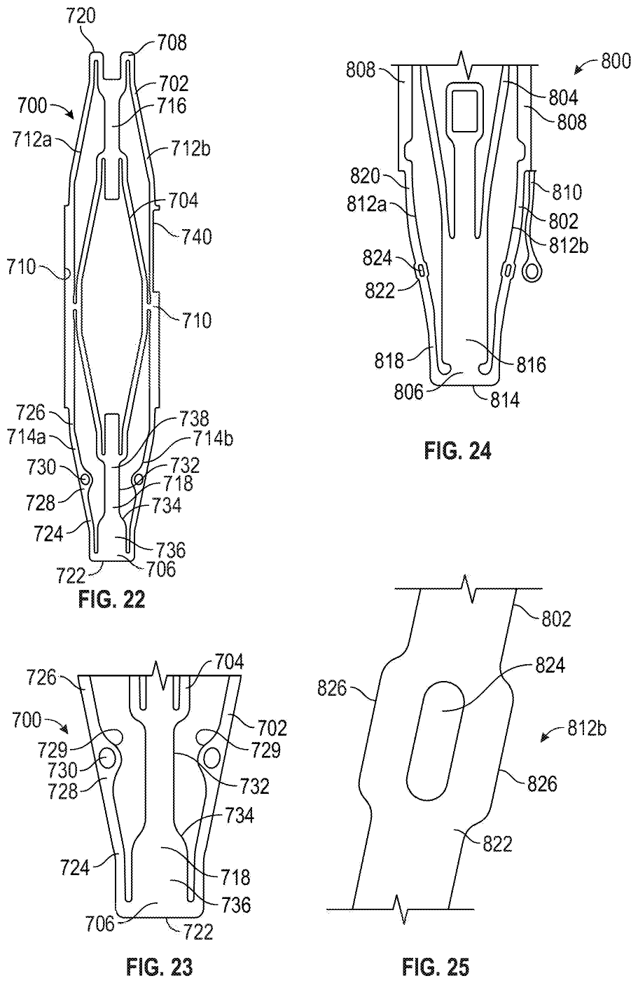

FIGS. 22 and 23 show a portion of a frame 700 according to another example in a radially compressed state. For the ease of illustration and discussion, FIGS. 22 and 23 illustrate a single first cell 702 and a corresponding second cell 704 of the frame 700. While only single first and second cells 702, 704 of the frame 700 are depicted, it should be appreciated that the frame 700 forms an annular structure having multiple cells 702, 704.

FIGS. 22 and 23 show the frame 700 can comprise first and second cells 702, 704, which form respective inflow and outflow apices 706, 708. The first and second cells 702, 704 are located between a pair of adjacent support posts 710 with one support post including a commissure support member 740 (for example, commissure support member 622) on one side of the cells (only one half of each support post is shown in FIG. 22). One or more of the support posts 710 can also include any one of the axial extensions described above.

Each first cell 702 can be formed by two upper struts 712a, 712b and two lower struts 714a, 714b. Each upper strut 712 is coupled on one end to a first axial post 716 and on the other end to a support post 710, while each lower strut 714 is coupled on one end to a second axial post 718 and on the other end the support post 710. The upper struts 712a, 712b can be part of an upper row of struts that defines the outflow end 720 of the frame 700, and the lower struts 714a, 714b can be part of a lower row of struts that defines the inflow end 722 of the frame 700.

As shown in FIGS. 22 and 23, one or more of the lower struts 714 of the frame 700 can have a similar configuration as the selected struts 644 of frame 600. Specifically, the lower struts 714 of frame 700, which can also be referred to as selected struts, can include an inflow end section 724 coupled to an inflow apex 706 of the frame 700, an outflow end section 726 coupled to a support post 710, and a midsection 728 framing an opening or aperture 730 radially extending therethrough. Each midsection 728 can also have a protrusion 729 (which can have a rounded edge) which protrudes into the opening formed by the first cells 702 in such a way that the protrusions have a width greater than a width of the inflow and outflow sections 724, 726.

One difference between the frame 600 and frame 700 are the respective indentations. In particular, rather than being formed to have the same or similar curvature as the protrusions 729 of the midsections 728 (for example, as indentations 654 do), indentations 732 of frame 700 are formed within and extend along a greater axial length of the longitudinal edges 734 of the posts 718. As best illustrated in FIG. 23, for instance, the second axial posts 718 comprise first end portions 736 that form the inflow apices 706 and second end portions 738 which extend from the inflow edge portions 736 to the minor vertices of the second cells 704. As shown in FIG. 23, the indentations 732 can be formed within the space created by the relatively narrow circumferential width of the second end portions 738 of the posts 718 relative to the inflow end portions 736 of the posts 718. The circumferential width of the second end sections 738 can be sufficiently narrow such that the indentations 732 are configured to receive the protrusions 729 of pair of adjacent selected struts 714. In some examples, the longer indentations 732 can be configured to receive relatively elongate and/or one or more protrusions of the selected struts 714, such as when a selected strut 714 comprise two or more protrusions 729 forming apertures 730. In alternative examples, the first and second end portions 736, 738 of posts 718 need not have different circumferential widths, but only a single width sufficiently narrow to prevent the midsections 728 from contacting the second axial posts 718 when the frame 700 is in a radially compressed state.

FIG. 24 shows an inflow end of a portion of a frame 800 in a radially compressed state, according to another example. For purposes of discussion, FIG. 24 depicts only a single set of first and second cells 802, 804 of the larger annular structure of the frame 800. As shown in FIG. 24, the first and second cells 802, 804 can form respective inflow apices 806 and outflow apices (not shown), and be situated between a pair of adjacent support posts 808 with one support post including an axial extension 810 (for example, axial extension 532). Any one of the commissure support members described herein can also extend and/or be formed within a support post 808.

Each first cell 802 can be formed by two upper struts and two lower struts 812a, 812b. The upper struts can be part of an upper row of struts that defines the outflow end of the frame 800, and the lower struts 812a, 812b can be part of a lower row of struts that defines the inflow end 814 of the frame 800. As shown in FIG. 24, the lower struts 812 are coupled on one end to one of two axially aligned posts 816 and on the other end to a support post 808.

The lower struts 812 of the frame 800, which can also be referred to as selected struts, can have a similar configuration as the selected struts 644 of frame 600 and selected struts 714 of frame 700. Each of the selected struts 812 of the frame 800, for instance, can comprise an inflow end section 818 coupled to an inflow apex 806 of the frame 800, an outflow section 820 coupled to a support post 808, and a midsection 822 framing an opening or aperture 824 radially extending therethrough.

In contrast to the selected struts 644 and selected struts 714, the midsection 822 of the selected struts 812 forms an elongated aperture 824 that can be generally rectangular in shape. The midsection 822 can have a pair of parallel thickened edges or protrusions 826 extending outwardly. Specifically, as shown in FIG. 25, which shows a magnified view of the midsection 822 of the selected struts 812b, the inner and outer edges of the struts 812 protrude outwardly into the opening formed by the first cells 802 (for example, toward a respective second cell 804 and/or post 816) and outwardly toward the inflow end 814 of the frame 800, respectively. As shown in FIGS. 24 and 25, the aperture 824 can also be rectangular or elongate and disposed between the rounded edges 826

One advantage of the selected struts 812 over the selected struts 644 and selected struts 714 is that the protrusions of the midsections 822 of struts 812 protrude outwardly to a lesser degree than the protrusions 649, 729 of the midsections 648, 728 of the struts 644, 714. In other words, the midsections 822 of the struts 812 have a narrower circumferential profile or width than the midsections 648, 728 of the selected struts 644, 714. This narrower width can, for example, allow the frame 800 to fully compress while avoiding contact between the midsections 822 of the struts 812 and the posts 816, absent indentations within the posts 816 and configured to receive the midsections 822.

Although FIGS. 22-25 show the selected struts of their respective frames 700, 800 as a pair of adjacent struts forming individual first cells 702, in some examples, the selected struts can be a pair of adjacent selected struts which form adjacent first cells 702 (for example, selected struts 644 of frame 600). In such examples, one or more struts within the row of lower struts forming the inflow end of the frame lack, an opening or aperture.

FIG. 26 illustrates another example of a prosthetic valve 900 in a radially expanded configuration. The prosthetic valve 900 in the illustrated example includes a frame 600. The frame 600 can include all of the components as previously described in connection with FIGS. 19-21 and therefore a detailed description of the frame 600 is not repeated here for the sake of brevity. Although illustrated and described as comprising frame 600, the prosthetic valve can comprise any frame described herein (for example, frame 102, frame 302, frame 400, frame 500, frame 600′, frame 600″, frame 600′″, frame 600″″, frame 700, and frame 800). Accordingly, the prosthetic valve 900 can comprise similar features and functionality as prosthetic valve 100, prosthetic valve 300, and prosthetic valve 656, as previously described.

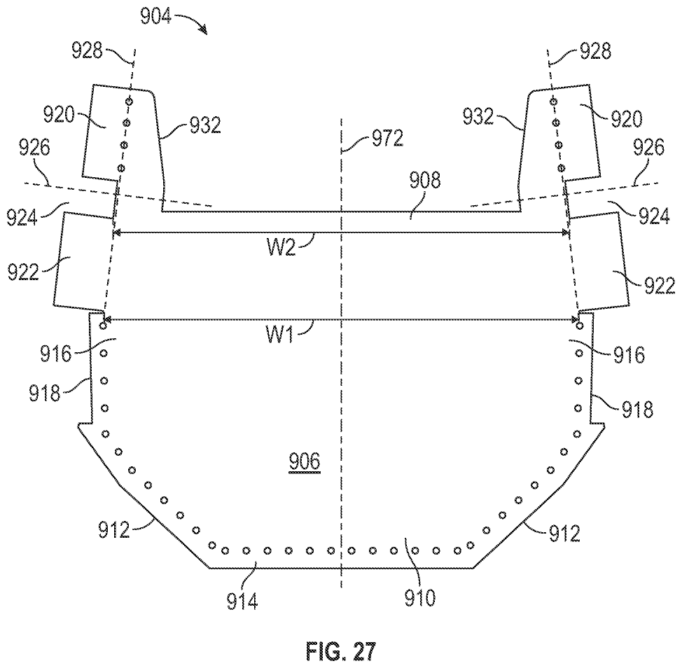

The prosthetic valve 900 includes a leaflet assembly 902 which is coupled to and supported inside the frame 600 and configured to regulate the flow of blood through the prosthetic valve 900 from the inflow end 602 to the outflow end 604. The leaflet assembly 902 can comprise a leaflet assembly comprising three leaflets 904 (a single leaflet is shown in FIG. 27) mounted within the frame 600 and arranged to collapse in a tricuspid arrangement. The leaflets 904 can be made of flexible material, such as from in whole or in part, biological material, bio-compatible synthetic materials, or other such materials. Suitable biological material, for instance, can include bovine pericardium (or pericardium from other sources). In some instances, the leaflet assembly 902 can include a greater or fewer number of leaflets.

As shown in FIG. 27, each leaflet 904 of the leaflet assembly 902 can include a main body 906, an outflow edge portion 908, and an inflow edge portion 910. The inflow edge portion 910 can comprise a pair of angled side edge portions 912 and an apex edge portion 914 extending between the angled side edge portions 912. The apex edge portion 914 and the side edge portions 912 can be straight or substantially straight such that the inflow edge portion 910 has a truncated V-shape. In other examples, the inflow edge portion 910 can be curved, such as in a U-shaped or a parabolic curve. Each leaflet 904 further includes sub-commissure edge portions 916 comprising axially extending side edges 918, each of which extends between a lower tab 922 of the leaflet and a respective angled side edge portion 912.

Each outflow edge portion 908 can extend between pairs of opposing upper and lower tabs 920, 922 disposed on opposite sides of the main body 906. The outflow edge portion 908 is configured to move radially inwardly to coapt with the other outflow edge portions 908 of adjacent leaflets under diastolic pressure and move radially outwardly toward the frame 600 under systolic pressure. As shown in FIG. 27, the upper and lower tabs 920, 922 on either side of the outflow edge portion 908 can be angled relative to a longitudinal axis 972 of the leaflet 904, which extends midway through the main body 906, from the apex edge portion 914 to the outflow edge portion 908. In particular, the upper and lower tabs 920, 922 can be angled relative to the longitudinal axis 972 at an angle greater than zero (that is, non-parallel). In this instance, the upper and lower tabs 920, 922 of each leaflet 904 can be said to be angled inwardly toward the longitudinal axis 972 of the leaflet. The upper and lower tabs 920, 922 can also be angled relative to the outflow edge portion 908 and sub-commissure edge portions 916, which can be generally perpendicular and parallel to the longitudinal axis 972 of the leaflet 904, respectively.

As illustrated in FIG. 27, when the upper and lower tabs 920, 922 are angled relative to the longitudinal axis 972 of the leaflet 904, the width of the main body 906 between the tabs 920, 922 narrows from the sub-commissure edge portions 916 to the outflow edge portion 908 of the leaflet 904. Specifically, a width W1 between the lower tabs 920 where the lower tabs meet the sub-commissure edge portions 916 is greater than a width W2 between the lower tabs 922 at or proximate the outflow edge portion 908 (that is, between the upper edge of the lower tabs 922 closest to a notch 924). As such, the width of the main body 906 progressively decreases from the sub-commissure edges 918 toward the outflow edge portion 908 of the leaflet 904. Inversely, the width between the tabs 920, 922 widen from the outflow edge portion 908 to the sub-commissure edge portions 916 such that the width of main body 906 progressively increases toward the sub-commissure edge portions 916 from the outflow edge portion 908.

Below each upper tab 920, there can be a notch 924 separating the upper tab 920 from the lower tab 922. An imaginary fold line 926 extends through the notch 924 and between each pair of upper and lower tabs 920, 922. As described in further detail below, each upper tab 920 can be folded over the fold line 926 and positioned against the lower tab 922 such that the tabs on each side of the main body 906 form a reinforced commissure tab or tab assembly. As shown in FIG. 27, because the upper and lower tabs 920, 922 are angled relative to the longitudinal axis of the leaflet 904, the fold line 926 is also angled at a non-zero angle relative to the longitudinal axis 972, outflow edge portion 908, and sub-commissure portions 916. This angle of the fold line 926 allows each upper tab 920 of the leaflets 904 when folded over, to be positioned against and overlap its corresponding lower tab 922 at a suitable angle for connecting the tabs to one another. In this case, the fold line 926 can also be perpendicular or substantially perpendicular to respective stitch lines 928 connecting the tabs 920, 922.

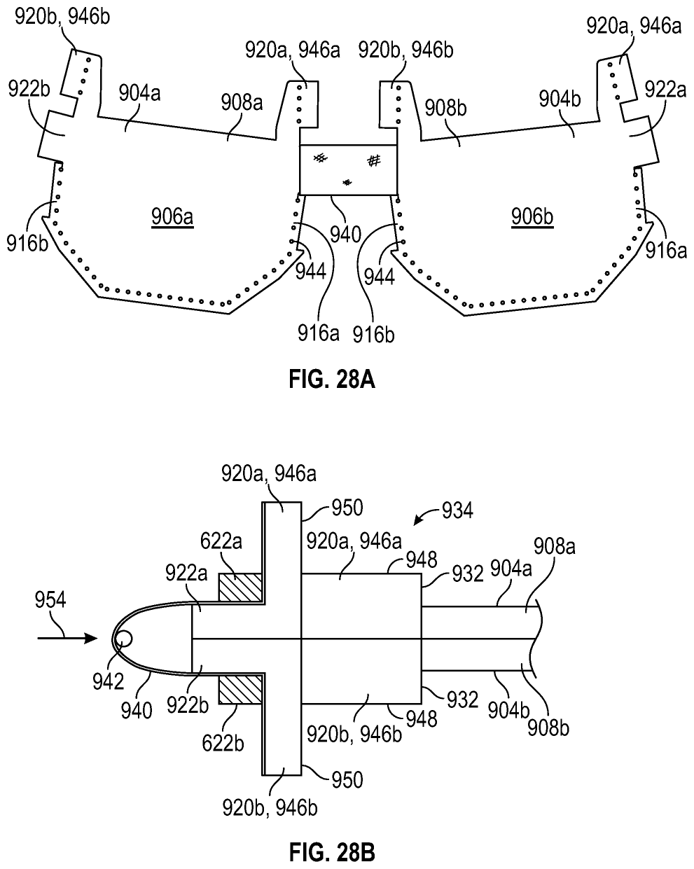

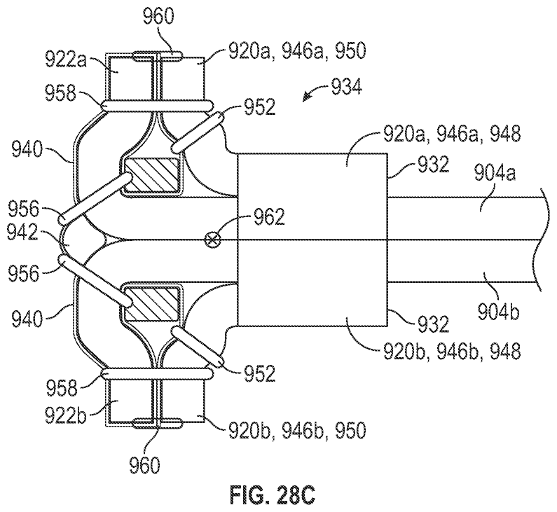

In forming a respective commissure tab, each upper tab 920 can be connected to its corresponding lower tab 922 via sutures extending along a stitch line 928 of the leaflets 904. In particular, once an upper tab 920 is folded over fold line 926, the upper and lower tabs 920, 922 can be connected to one another along the stitch line 928 by sutures forming in-and-out and/or whip stitches extending through the upper tab 920 and through lower tab 922. The commissure tabs formed of the upper and lower tabs 920, 922 can be paired with the commissure tabs of adjacent leaflets 904 to form a leaflet assembly and respective leaflet commissures 934 (for example, see FIGS. 26 and 28A-28C). In some instances, to form a leaflet commissure 934, a stitch line 928 of one commissure tab formed by the upper and lower tabs 920, 922 can be a stitch line in which the one commissure tab is stitched to a respective commissure tab of an adjacent leaflet 904 and/or a connector coupled to a respective commissure tab of the adjacent leaflet (for example, connector 940). These stitch lines 928 of the leaflet commissures can also serve as the location at which each leaflet commissure is coupled to a respective commissure support member 622 of the frame 600.

As shown in FIG. 26, each leaflet commissure 934 formed of the upper and lower tabs 920, 922 can have an inflow end 936 nearest a respective sub-commissure edge portion 916 and an outflow end 938 nearest the outflow edge portion 908 of the leaflet 904. When the upper tab 920 is folded over and sutured to its corresponding lower tab 922, the material of the upper tab 920 located on the inside of the stitch line 928 can also form an inner edge 932 of the leaflet commissures 934. The inner edge 932 can extend from the inflow end 936 to the outflow end 938 of its respective commissure 934.

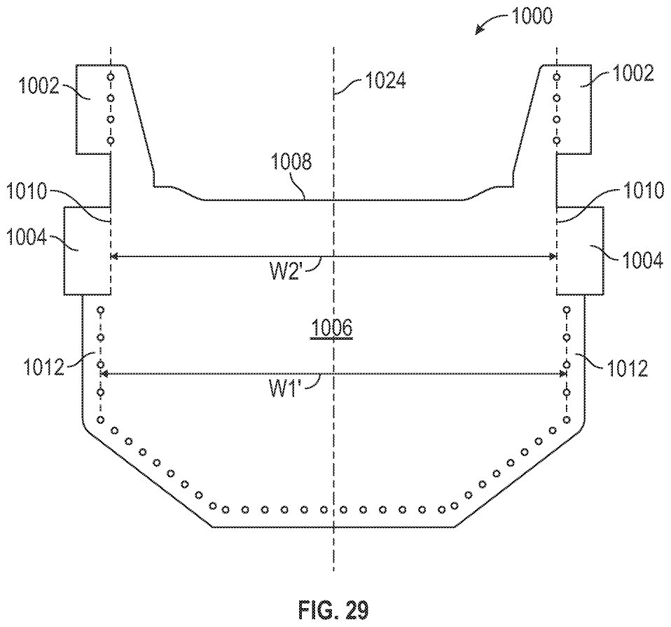

As illustrated in FIG. 27, each stitch line 928 can be parallel to its respective upper and lower tabs 920, 922 and therefore, angled relative to the longitudinal axis 972 of the leaflet 904 at a non-zero angle. In particular, the stitch lines 928 track an inner edge of their respective upper and lower tabs 920, 922 and the width of the main body 906. Consequently, prior to being mounted on the frame 600, the length of the outflow edge portion 908 of the leaflets 904, that is, the outflow edge portion 908 between the tabs 920, 922 and stitch lines 928, is relatively shorter than an outflow edge of a leaflet having longitudinally oriented tabs (FIG. 29), for example, due to the narrowing of and comparatively shortened distance between the tabs 920, 922.

FIGS. 28A-28C illustrate in further detail the assembly of the leaflet assembly 902 of the prosthetic valve 900, including the leaflets 904. Referring to FIG. 28A, for instance, in particular examples, the leaflet assembly 902 can be formed by connecting a flexible connector 940 to a pair of leaflets 904a, 904b at a lower tab 922a of leaflet 904a and lower tab 922b of the leaflet 904b. The flexible connector 940 can be connected to the lower tabs 922a, 922b with sutures. The flexible connector 940 can comprise, for example, a piece of fabric (for example, PET fabric). A wedge element 942 (FIGS. 28B and 28C) can be connected to one side of the flexible connector 940. The wedge element 942 can comprise, for example, a relatively heavy gauge suture, such as a braided suture (for example, an Ethibond suture), or a piece of fabric. At this stage, a reinforcing strip (not shown) can be connected to the inflow edge portion 910 of each leaflet 904a, 904b. A reinforcing strip can, for example, protect the leaflet material at the inflow edge portion 910 from tears and be made of a tear resistant material that is biocompatible, such as a polyethylene terephthalate (PET). In some examples, a variety of other synthetic or natural materials may be used.

A third leaflet 904 (not shown) can be similarly coupled to leaflets 904a, 904b by connecting a second connector 940 to the lower tab 922b of the leaflet 904a and a corresponding lower tab of the third leaflet and connecting a third connector 940 to the lower tab 922a of the leaflet 904b and the other lower tab of the third leaflet, thereby forming the leaflet assembly 902 of three leaflets (for example, FIG. 26) coupled to each other with respective connectors 940. It should be understood that the leaflet assembly can include additional leaflets coupled to each other with additional connectors 940.

The adjacent axially extending sub-commissure edge portions 916a, 916b of adjacent leaflets can be connected to each other with sutures, such as along a series of markings 944. The sutures can, for instance, form in-and-out stitches or whip stitches that extend through adjacent sub-commissure edges 916a, 916b.

As mentioned, the upper tabs 920a, 920b of each leaflet 904 can be folded downwardly against their corresponding lower tabs 922a, 922b (for example, over fold line 926, FIG. 27). For example, referring to FIG. 28A, the upper tab 920a of the leaflet 904a can be folded downwardly against the lower tab 922a of the leaflet 904a on the same side of the leaflet as the connector 940. In this manner, the upper tab 920a can partially overlap the lower tab 922a and the portion of the connector 940 situated between the lower tab 922a and the upper tab 920a. Similarly, the upper tab 920b of the leaflet 904b can be folded downwardly against the lower tab 922b of the leaflet 904b and the portion of the connector 940 therebetween.

After folding the upper tabs 920a, 920b, tab portions 946a, 946b of each upper tab 920a, 920b can be folded lengthwise along a vertical fold axis to form an L-shape having an inner portion 948 and an outer portion 950 (for example, FIG. 28B). The inner portion 948 can contact the inner surface of the leaflet and the outer portion 950 can contact the connector 940. The outer portions 950 can be sutured to the connector 940, such as with sutures 952 (FIG. 28C).

Once the upper tabs 920a, 920b are folded downwardly and the tab portions 946a, 946b, folded lengthwise, the resulting leaflet assembly 902 can be positioned within the frame 600. For each leaflet 904, the relatively narrower outflow edge portions 908 and the portions of the main body 906 between respective stich lines 928 (FIG. 27) and commissures 934 (FIGS. 26 and 28B-28C) can be stretched to position each respective commissure 934 adjacent a commissure support member 622 (FIGS. 20-21 and 26). When positioning the commissures 934 against the commissure support members 622 of the frame 600, each commissure 934 (including upper and lower 920, 922 tabs from two adjacent leaflets) is pulled and deformed such that each pair of upper and lower tabs 920, 922 is pivoted from an angled orientation (in which stitch line 928 is angled relative to the longitudinal axis 972) to a vertical orientation (in which stitch line 928 is parallel to the longitudinal axis 972). This causes the width W2 between the stitch lines 928 at the outflow ends 938 of the commissures 934 to become equal or substantially equal to the width W1 between the stitch lines 928 at the inflow ends 936 of the commissures 934. As described further herein, these relatively stretched portions of the main body 906 between the tabs 920, 922 and along the outflow edge portion 908 can remove slack along the outflow edge 908 and pre-tension the leaflets.

Referring to FIG. 28B, the leaflet commissure 934 formed by a connector 940, the tabs 922a, 920a of the leaflet 904a, and tabs 922b, 920b of the leaflet 904b can be coupled to the commissure support member 622 of a frame 600 as follows. As shown in FIG. 28B, the connector 940 and the lower tabs 922a, 922b can be inserted through a window 624 (FIGS. 19-20) defined by struts 622a, 622b of the commissure support member 622, while the upper tabs 920a, 920b remain inside the frame. The commissure 934 is then pressed inwardly at the wedge element 942 (in the direction of arrow 954) such that the outer portion 950 of the tab portion 946a and a portion of the connector 940 are against the frame on one side of the window 624 and the outer portion 950 of the tab portion 946b and a portion of the connector 940 are against the frame on the other side of the window 624.

As shown in FIG. 28C, the pressing of the commissure 934 also causes the lower tab 922a and a portion of the connector 940 to fold around the strut 622a on the outside of the frame opposite the outer portion 950 of the upper tab 920a, and the lower tab 922b and a portion of the connector 940 to fold around the strut 622b on the outside of the frame 600 opposite the outer portion 950 of the upper tab 920b. A pair of suture lines 956 can be formed to retain the lower tabs 922a, 922b against the frame. Each suture line 956 extends through the connector 940, a lower tab, the wedge element 942, and another portion of the connector 940.

Each lower tab 922a, 922b can be secured to a corresponding upper tab 920a, 920b with a primary suture line 958. Each suture line 958 extends through one layer of the connector 940, a lower tab 922a, 922b, another layer of the connector 940, another layer of the connector 940, and the outer portion 950 of the upper tab 920a, 920b. The end portions of the suture material used to form the primary suture lines 958 (or separate sutures) can be used to form whip stitches 960 at the adjacent outer edges of the tabs 922a, 920a and at the adjacent outer edges of the tabs 922b, 920b. A first set of stitches 960 can extend through the tabs 922a, 920a and two layers of the connector 940 between the tabs 922a, 920a, and a second set of stitches can extend through the tabs 922b, 920b and two layers of the connector 940 between the tabs 922b, 920b.

During diastole and systole, the leaflets 904a, 904b can articulate primarily at inner edges 932 of the folded inner portions 948. However, when the prosthetic valve is radially compressed to a delivery state, the relatively higher forces acting on the leaflets can cause the leaflets to splay apart about a longitudinal axis 962, allowing for a smaller crimped diameter.

The remaining commissures 934 of the leaflet assembly can be coupled to respective commissure windows 624 of the frame 600 in the same manner as described above. Further details of the method for forming the commissure tab assemblies and coupling them to the frame are disclosed in U.S. Pat. No. 9,393,110, which is incorporated herein by reference. Although described as coupling leaflets 904 to frame 600, it should be appreciated that techniques described in reference to FIGS. 28A-28C can be used to assemble any of the prosthetic valves described herein. It should also be noted that FIGS. 28A-28C show one exemplary technique for coupling the commissures of a leaflet assembly to a frame. Other techniques, methods, and mechanisms can be used for coupling the commissures of the leaflet assembly to the frame 600, such as any of those disclosed in U.S. Pat. No. 9,393,110, U.S. Publication No. 2018/0325665, or U.S. Application No. 63/003,085, filed Mar. 31, 2020, which are incorporated herein by reference.

It should also be appreciated, that the remaining portions of the leaflets 904, including the inflow edge portions 910 can be coupled to the frame 600 in any manner described herein. For instance, the leaflets 904 can be coupled to the frame 600 in a similar manner as described for leaflets 660 of prosthetic valve 656 (FIG. 21). For example, as show in FIG. 26, each inflow edge portion 910 can be coupled to one or more selected struts 644 and/or axial extensions 626 having respective apertures 646, 638. The angled edge portions 912, for instance, can be coupled to selected struts 644 forming first cells 610 and inflow apices 612, and which extend between the inflow apices 612 and the support posts 620 via sutures 966. Moreover, the apex edge portions 914 can extend between adjacent inflow apices 612 and be secured or anchored to the frame 600 via an axial extension 626 such that each leaflet extends between respective inflow apices 612 and is directly coupled to the frame 600 at a corresponding axial extension 626 via sutures 968 extending through aperture 638. The axially extending sub-commissure edge portions 916 of the leaflets 904 can extend along the support posts 620. In some examples, the adjacent sub-commissure edge portions 916 are coupled to the support posts 620.

Sutured to the inflow edge portions 910 of the leaflets 904 can also be a fabric connecting skirt 964 used to connect the apex edge portion 914, angled side edge portion 912, and/or axially extending sub-commissure edge portion 916 to a corresponding selected strut 644. The connecting skirt 964, for instance, can be coupled to the inflow edge portions 910 via sutures 970. The prosthetic heart valve 900 can also include an outer skirt (not shown) mounted to the outer surface of the frame 600 (for example, outer skirt 374 and outer skirt 548) and/or be coupled to the inflow edge portion 910 of one or more leaflets 904 as previously described.

As described herein, when the leaflets 904 are mounted to the frame 600, each leaflet commissure 934 can be deformed and coupled to a respective support member 622 in a longitudinal and axial direction. In particular, each pair of upper and lower tabs 920, 922 of a respective leaflet 904 and corresponding stitch line 928 are pivoted from an angled orientation in which the stitch line 928 is angled relative to the longitudinal axis 972 of the leaflet 904 (FIG. 27), to a vertical orientation in which the stich line 928 is parallel to the longitudinal axis 972. When coupled to the frame 600 in this way, the relatively narrow outflow edge portions 908 and the portions of the main body 906 between the stich lines 928 are stretched relative to the other portions of the leaflets 904 (for example, the inflow edge portion 910). This relative stretching occurs as the width W2 between the stitch lines 928 at the outflow ends 938 of the commissures 934 becomes equal or substantially equal to the width W1 between the stitch lines 928 at the inflow ends 936 of the commissures 934 (FIG. 27). Due to the reduced slack along the outflow edge portions 908, during systole, the stretched portions of the main body 906 between the tabs 920, 922 and outflow edge portion 908 of each leaflet 904 become tensioned between respective leaflet commissures 934 and support members 622 of the frame 600 as the leaflets 904 resist further radially outwardly movement beyond a tensioned or taut state.

Accordingly, the tension across the leaflets can be at its greatest at those portions of the leaflets which are most stretched between their respective pair of commissures 934. That is to say, the tension across the leaflets 904 when the leaflets are in an opened state increases with the decreasing width between the upper and lower tabs 920, 922 and stich lines 928 (for example, width W1 to width W2 in FIG. 27), due to the increasing stretch at the relatively narrowest portions of the leaflets. As such, the tension across the leaflets 904 can progressively increase from a plane intersecting the inflow ends 936 of the commissures 934 to the outer most edge of the outflow edge portions 908. In other examples, however, there can be little to no tension across the leaflets 904 at the inflow ends 936 at the commissures 934. Rather, in such examples, tension across the leaflets 904 can extend from a plane intersecting the leaflets where tensioned is formed between the inflow and outflow ends 936 of the commissures 934, to the outflow edge portions 908.

In some examples, when the leaflets 904 are coupled to the frame 600, the width W2 between the stitch lines 928 at the outflow ends 938 of the commissures 934 need not be equal to the width W1 between the stitch lines 928 at the inflow ends 936 of the commissures 934. For example, in some instances, the outflow edge portions 908 and the portions of the main body 906 between the stich lines 928 can be stretched relative to the other portions of the leaflets 904 such that tension is still created across the leaflets 904, but where the width W2 between the stitch lines 928 at the outflow ends 938 is still less than the width W1 between the stitch lines 928 at the inflow ends 936 of the commissures 934. In such examples, segments of one or more of the stitch lines 928 can be angled inwardly toward the longitudinal axis of the frame 600.

The increasing tension across the leaflets 904 can result in a gradual decrease in the effective orifice area of the valve 900 and an outflow channel of the leaflets that tapers toward the outflow edge portions 908 and longitudinal axis of the frame 600. Specifically, an inner diameter of the leaflet assembly 902 can progressively decrease in diameter as the tension progressively increases toward the outflow edge portions 908 of the leaflets 904. This progressive decrease in the inner diameter of the leaflets 904 can define the tapered outflow channel of the valve 900 which tapers along the longitudinal axis of the frame 600 while the frame 600 remains cylindrical.

Since the outflow channel can be created by tension across the leaflets 904, in some examples, the tapered outflow channel can extend between the inflow and outflow ends 936, 938 of the commissures 934 as the inner diameter of the leaflets narrows toward the outflow edge portions 908, from the inflow ends 936 of the commissures 934. As such, an inner diameter of the leaflet assembly 902 and outflow channel at the inflow ends 936 of the commissures 934 can be greater than the inner diameter of the leaflet assembly and outflow channel at the outflow edge portions 908. In other examples, the tapered outflow channel can be created by tension across the leaflets 904 which extends to the outflow edge portions 908, but from a plane intersecting the leaflets between the inflow and outflow ends 936, 938 of the commissures 934.

The tapered outflow channel of the leaflets 904 can provide particular advantages over conventional leaflets. A problem with conventional leaflets, for instance, is that conventional leaflets, such as those leaflets having tabs parallel to a longitudinal axis of the leaflets, can form a generally cylindrical outflow channel. Flow disturbances created by flow separation at the outlet of a cylindrical outflow channel, for example, as the laminar flow across a valve turns into turbulent flow, can cause leaflet flutter at the outlets. This fluttering of the leaflets can result in fatigue failure over time. By comparison, the formation of the tapered outflow channel by the tensioned leaflets 904 can reduce the turbulent flow at the outflow edge portions 908, minimizing the flow disturbances which can cause the leaflets to flutter.

To illustrate, the following description in connection with FIGS. 29-31 provides a comparison between conventional leaflets and those leaflets 904 described herein. For example, FIG. 29 shows a conventional leaflet 1000 with longitudinally oriented tabs. The leaflet 1000 includes pairs of upper and lower tabs 1002, 1004 located on opposing sides of a main body 1006 and outflow edge portion 1008. In comparison to leaflet 904 (FIG. 27), the upper and lower tabs 1002, 1004 are generally parallel to a longitudinal axis 1024 of the leaflet 1000 such that the width W2′ between the tabs 1002, 1004 is unchanged. The upper and lower tabs 1002, 1004 can also have inner edges 1010 offset from the sub-commissure edge portions 1012. For instance, a width W1′ between the opposing sub-commissure edge portions 1006 is greater than a width W2′ between the inner edges 1010 of the lower tabs 1004. For illustrative purposes, the width W1′ between the opposing sub-commissure edge portions 1012 can be the distance between markings 1014 indicating where the sub-commissure edge portions 1012 are coupled to a frame.

The upper tabs 1002 of the leaflet 1000 can be folded over and sutured to corresponding lower tabs 1004 to form respective commissure tabs. Each commissure tab formed of the upper and lower tabs 1002, 1004 can be coupled to a respective commissure tab of an adjacent leaflet 1000 to form leaflet commissures and a corresponding leaflet assembly. The upper and lower tabs 1002, 1004 can be sutured to one another and/or a commissure tab of an adjacent leaflet 1000 along a line tracking the inner edges 1010 of the tabs parallel to the longitudinal axis 1024 of the leaflet 1000. When coupled to a frame, the leaflet commissures formed of upper and lower tabs 1002, 1004 and adjacent leaflets 1000 are coupled to the frame in a longitudinal fashion such that the stitching connecting the commissure tabs of adjacent leaflets is parallel to the longitudinal axis of the frame and extends in an axial direction. In this instance, the offset arrangement between the inner edges 1010 and sub-commissure edge portions 1012 creates a step-like transition where the sub-commissure edge portions 1012 meet the leaflet commissures. As the leaflets 1000 open under systolic pressure, the outflow edge portions 1008 of the leaflets collectively form a generally cylindrical outflow channel and slack along the nontensioned outflow edge portions 1008 can create the flow separation that causes the flow disturbances and leaflet fluttering briefly described above.

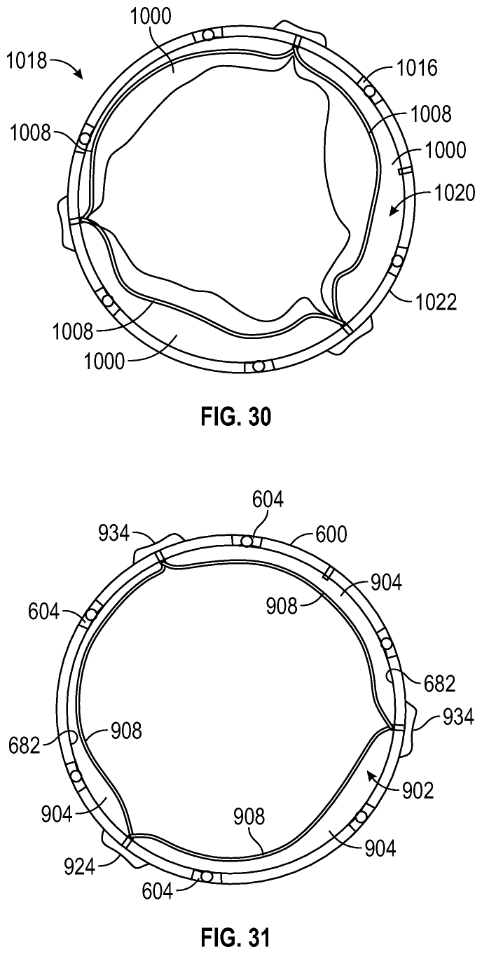

As a particular example, FIG. 30 depicts an outflow end 1016 of a prosthetic valve 1018, that includes a valvular structure 1020 constructed of three leaflets 1000 shown in FIG. 29. The prosthetic valve 1018 is shown from a downward view along a longitudinal axis extending through the center of the valve frame 1022 (that is, from the outflow end to the inflow end of the valve 1018). As depicted in FIG. 30, during systole, when a pressure gradient across the valve forces the leaflets 1000 open, the pressure gradient can cause the outflow edge portions 1008 to flutter due to the slack across each outflow edge portion 1008. This fluttering can cause a disturbance in the blood flow across the valve 1018.

Unlike the parallel tabs 1002, 1004 of leaflets 1000, the angled upper and lower tabs 920, 922 of the leaflets 904 create tension across the outflow edge portions 908 and form the tapered outflow channel to avoid such slack and fluttering. By way of example, FIG. 31 shows the outflow end portion 604 of the prosthetic valve 900 along the longitudinal axis of the frame 600 and depicts the tension across leaflet assembly 902 when the leaflets 904 are opened under systolic pressure. As blood flows from the inflow end 602 to the outflow end 604 of the frame 600, the outflow edge portions 908 of the leaflets 904 move radially outwardly toward an inner surface 682 of the frame 600 due to the pressure gradient across the valve 900. This radially outwardly movement creates tension across those relatively stretched portions of the leaflets 904 stretched during assembly of the valve 900 and form the tapered outflow channel. As such, the tension across the leaflets 904 extends 360 degrees around the leaflet assembly 902 as the relatively stretched portions of the outflow edge portions 908 and main bodies 906 become fully tensioned (for example, with little to no slack in the outflow edge portions) and resist further radially outwardly movement toward the inner surface 682 of the frame 600.

As illustrated in FIG. 31, the tension across the outflow edge portions 908 of the leaflets 904 can also offset the outflow edge portions 908 of the leaflets radially inwardly from the inner surface 682 of the frame 600 as the tapered outflow channel is formed and the inner diameter of the leaflets 904 progressively decreases toward the outflow edge portions 908. For instance, as the leaflets 904 resist further radial movement outward and become tensioned, the outflow edge portions 908 form a radial gap between the outflow edge portions 908 and inner surface 682 of the frame 600. This radial gap, among other things, can improve the durability and longevity of the leaflets 904 by eliminating or at least limiting contact between the leaflets 904 and the inner surface 682 of the frame 600 and thereby limiting the potential wear and damage that can otherwise be caused when leaflets are allowed to contact the frame.

FIG. 32 illustrates a delivery apparatus 1100, according to one example, adapted to deliver a prosthetic heart valve 1102 described herein (for example, prosthetic heart valves 100, 300, and 656). The prosthetic valve 1102 can be releasably coupled to the delivery apparatus 1100. It should be understood that the delivery apparatus 1100 can be used to implant prosthetic devices other than prosthetic valves, such as stents or grafts.

The delivery apparatus 1100 in the illustrated example generally includes a handle 1104, an outer elongated shaft 1106 extending distally from the handle 1104 and at least one actuator assembly 1108 extending distally through the outer shaft 1106. The delivery apparatus 1100 can also include an elongated inner shaft 1120 extending distally from the handle 1104 through the outer shaft 1106. A nosecone 1122 can be connected to the distal end of the inner shaft 1120. The at least one actuator assembly 1108 can be configured to radially expand and/or radially collapse the prosthetic valve 1102 when actuated.

For the purpose of illustration, the illustrated example shows only two actuator assemblies 1108. However, it should be understood that one actuator assembly 1108 can be provided for each actuator member on the prosthetic valve 1102. For example, three actuator assemblies 1108 can be provided for a prosthetic valve 1102 having three actuators. In other examples, however, any greater or fewer number of actuator assemblies can be present. In the examples of FIGS. 1, 9, 15, 21, and 26, the prosthetic valves have six actuator members (for example, actuator members 158, 382), in which case the delivery apparatus 1100 can have six actuator assemblies 1108.

The distal end portion 1110 of the shaft 1106 can be sized and shaped to house the prosthetic valve 1102 in a radially compressed, delivery state during delivery of the prosthetic valve through, for example, the vasculature of a patient. In this way, the distal end portion 1110 functions as a delivery sheath or capsule for the prosthetic valve during delivery.

The actuator assemblies 1108 can be releasably coupled to the prosthetic valve 1102. For instance, in the illustrated example, each actuator assembly 1108 can be coupled to a respective actuator member of the prosthetic valve 1102. Each actuator assembly 1108 can comprise a support tube, an actuator member, and optionally a locking tool. When actuated, the actuator assembly can transmit pushing and/or pulling forces to portions of the prosthetic valve to radially expand and collapse the prosthetic valve as previously described. The actuator assemblies 1108 can be at least partially disposed radially within, and extend axially through, one or more lumens of the outer shaft 1106. For instance, the actuator assemblies 1108 can extend through a central lumen of the shaft 1106 or through separate respective lumens formed in the shaft 1106.

The actuator member of each actuator assembly 1108 can be releasably coupled to a respective actuator member of the prosthetic valve (for example, actuator member 158 or 382). The support tube of each actuator assembly 1108 can abut an adjacent portion of the frame of the prosthetic valve, such as an outflow apex (for example, apex 114 or 316). In this manner, during valve expansion, the support tubes can prevent movement of the outflow end of the prosthetic valve relative to the delivery apparatus while the actuator members of the actuator assemblies 1108 can actuate the actuator members of the prosthetic valve and cause the inflow end of the prosthetic valve to move toward the outflow end of the prosthetic valve.

The handle 1104 of the delivery apparatus 1100 can include one or more control mechanisms (for example, knobs or other actuating mechanisms) for controlling different components of the delivery apparatus 1100 in order to expand and/or deploy the prosthetic valve 1102. For instance, in the illustrated example, the handle 1104 comprises first, second, and third knobs 1112, 1114, and 1116.

The first knob 1112 can be a rotatable knob configured to produce axial movement of the outer shaft 1106 relative to the prosthetic valve 1102 in the distal and/or proximal directions in order deploy the prosthetic valve from the delivery sheath 1110 once the prosthetic valve has been advanced to a location at or adjacent the desired site of implantation within a patient. For instance, rotation of the first knob 1112 in a first direction (for example, clockwise) can retract the sheath 1110 proximally relative to the prosthetic valve 1102 and rotation of the first knob 1112 in a second direction (for example, counterclockwise) can advance the sheath 1110 distally. In other examples, the first knob 1112 can actuated by sliding or moving the knob 1112 axially, such as puling and/or pushing the knob. In still further examples, actuations of the first knob 1112, such as by rotation or sliding the first knob 1112, can produce axial movement of the actuator assemblies 1108 and thereby the prosthetic valve 1102 relative to the delivery sheath 1110 to advance the prosthetic valve distally from the sheath 1110.

The second knob 1114 can be a rotatable knob configured to produce radial expansion and/or contraction of the prosthetic valve 1102. For instance, rotation of the second knob 1114 can move the actuator members and the support tubes of actuator assemblies 1108 axially relative to one another. The actuator members of assemblies 1108 in turn cause corresponding movement of the actuator members (for example, members 158, 382) of the prosthetic valve. Rotation of the second knob 1114 in a first direction (for example, clockwise) can radially expand the prosthetic valve 1102 and rotation of the second knob 1114 in a second direction (for example, counterclockwise) can radially collapse the prosthetic valve 1102. In other examples, the second knob 1114 can be actuated by sliding or moving the knob 1114 axially, such as pulling and/or pushing the knob.

The third knob 1116 can be a rotatable knob configured to retain the prosthetic heart valve 1102 in an expanded state. For instance, the third knob 1116 can be operatively connected to a proximal end portion of the locking tool of each actuator assembly 1108. Rotation of the third knob 1116 in a first direction (for example, clockwise) can rotate each locking tool to advance the locking nuts to their distal positions to resist radial compression of the frame of the prosthetic valve. Rotation of the knob 1116 in the opposite direction (for example, counterclockwise) can rotate each locking tool in the opposite direction to decouple each locking tool from the prosthetic valve 1102. In other examples, the third knob 1116 can be actuated by sliding or moving the third knob 1116 axially, such as pulling and/or pushing the knob. In some examples, the prosthetic valve can be self-locking, in which case a locking tool is not required. For example, the frame of the prosthetic valve can include locking features that automatically engage the actuator members of the prosthetic valve to resist radial compression of the prosthetic valve after it is expanded, such as disclosed in U.S. Application Nos. 63/085,947, 63/138,599, and 63/179,766.

Although not shown, the handle 1104 can include a fourth rotatable knob operative connected to a proximal end portion of each actuator member. The fourth knob can be configured to rotate each actuator member, upon rotation of the knob, to unscrew each actuator member from the proximal portion of a respective actuator. As described above, once the locking tools and the actuator members are uncoupled from the prosthetic valve 1102, they can be removed from the patient.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a delivery capsule to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat, radiation, ethylene oxide, hydrogen peroxide, etc.) to ensure they are safe for use with patients, and any of the methods herein can include sterilization of the associated system, device, apparatus, etc. (for example, with heat, radiation, ethylene oxide, hydrogen peroxide, etc.) as one of the steps of the method.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1: A prosthetic heart valve comprising: a radially expandable frame comprising an outflow end and an inflow end; and a plurality of valve leaflets disposed within and coupled to the frame, each leaflet comprising a main body having an outflow edge portion and an inflow edge portion, wherein the leaflets are configured to move between an open state allowing blood to flow through the frame from the inflow end to the outflow end and a closed state in which the outflow edge portions coapt with each other and block the flow of blood through the frame from the outlet end to the inlet end, wherein the inflow edge portion of each leaflet includes a movable portion that can move radially inwardly when the leaflets move to the closed state to assist with the coaptation of the outflow edge portions of the leaflets and radially outwardly when the leaflets move to the open state.

Example 2: The prosthetic heart valve of any example herein, particularly example 1, wherein the frame comprises a plurality of inflow apices and the movable portion of each leaflet inflow edge portion comprises an apex edge portion that extends between a pair of adjacent inflow apices.

Example 3: The prosthetic heart valve of any example herein, particularly example 1, further comprising an outer skirt mounted to an outer surface of the frame, wherein the inflow edge portion of each leaflet is coupled to an inflow edge of the outer skirt such that the movable portion of each leaflet inflow edge portion and the inflow edge of the outer skirt are configured to move radially inwardly relative to the frame when the leaflets moved to the closed state.

Example 4: The prosthetic heart valve of any example herein, particularly example 2, further comprising an outer skirt mounted to an outer surface of the frame, wherein the apex edge portion of each leaflet is coupled to an inflow edge of the outer skirt such that the apex edge portion of each leaflet and the inflow edge of the outer skirt are configured to move radially inwardly relative to the frame when the leaflets move to the closed state.

Example 5: The prosthetic heart valve of any example herein, particularly any one of examples 1-4, wherein the moveable portion of each leaflet inflow edge portion is radially spaced from an inner surface of the frame by a first distance when the leaflets are in the open state and radially spaced from the inner surface of the frame by a second distance when the leaflets are in the closed state, wherein the second distance is greater than the first distance.

Example 6: The prosthetic heart valve of any example herein, particularly any one of examples 1-5, wherein the moveable portions of the leaflet inflow edge portions are unsupported by the frame.

Example 7: The prosthetic heart valve of any example herein, particularly any one of examples 1-5, wherein the frame comprises a plurality of cantilevered struts, wherein the movable portions of the leaflet inflow edge portions are connected to the cantilevered struts, which are configured to move radially inwardly when the leaflets move to the closed state and radially outwardly when the leaflets move to the open state.

Example 8: The prosthetic heart valve of any example herein, particularly example 7, when combined with any of examples 3-4, wherein the inflow edge of the outskirt is connected to the cantilevered struts.

Example 9: The prosthetic heart valve of any example herein, particularly example 2, wherein each two adjacent inflow apices forms a circumferential gap therebetween, and wherein the moveable portion of each leaflet inflow edge portion extends between every other circumferential gap.

Example 10: The prosthetic heart valve of any example herein, particularly example 9, wherein the frame comprises six inflow apices.

Example 11: The prosthetic heart valve of any example herein, particularly any one of examples 1-10, wherein the frame comprises six outflow apices.

Example 12: The prosthetic heart valve of any example herein, particularly any one of examples 1-11, wherein the plurality of leaflets comprises three leaflets.

Example 13: The prosthetic heart valve of any example herein, particularly any one of examples 1-12, the frame further comprising a commissure support member disposed between one or more adjacent pairs of outflow apices of the frame.

Example 14: The prosthetic heart valve of any example herein, particularly any one of examples 1-13, the frame further comprising a commissure support member disposed between each pair of adjacent outflow apices of the frame.

Example 15: The prosthetic heart valve of any example herein, particularly any one of examples 13-14, wherein one or more commissure support members comprise a first commissure arm, a second commissure arm, and an opening therebetween configured to receive a commissure formed by two adjacent leaflets.

Example 16: The prosthetic heart valve of any example herein, particularly any one of examples 1-15, the frame further comprising a plurality of circumferentially disposed cells.

Example 17: The prosthetic heart valve of any example herein, particularly example 16, wherein each of the cells forms an axially-extending elliptical shape.

Example 18: The prosthetic heart valve of any example herein, particularly any one of examples 16-17, wherein the cells form outflow and inflow apices of the frame.

Example 19: The prosthetic heart valve of any example herein, particularly any one of examples 16-18, wherein each cell is an outer cell, each outer cell having an inner cell disposed within an outer perimeter of the outer cell.

Example 20: The prosthetic heart valve of any example herein, particularly any one of examples 16-19, wherein the cells extend from the inflow end to the outflow end of the frame.

Example 21: The prosthetic heart valve of any example herein, particularly any one of examples 1-20, the frame further comprising a plurality of actuator members configured to produce radial expansion of the frame.

Example 22: The prosthetic heart valve of any example herein, particularly example 21, wherein the frame comprises a plurality of axially extending first posts and a plurality of axially second posts, wherein each actuator member extends through one of the first posts and one of the second posts.

Example 23: The prosthetic heart valve of any example herein, particularly example 22, wherein each actuator member comprising a threaded rod.

Example 24: The prosthetic heart valve of any example herein, particularly example 23, wherein each rod is configured to rotate such that the first and second posts move axially toward one another and radially expand the frame.

Example 25: The prosthetic heart valve of any example herein, particularly any one of examples 22-24, wherein the first posts and the second posts are configured to contact each other when the frame is radially expanded to prevent overexpansion of the frame.

Example 26: The prosthetic heart valve of any example herein, particularly any one of examples 23-24, wherein each threaded rod has external threads that engage internal threads of one of the first posts and/or one of the second posts.

Example 27: A prosthetic heart valve comprising: a radially expandable frame comprising an outflow end portion, an inflow end portion, a central longitudinal axis extending from the inflow end portion to the outflow end portion, a plurality of outflow and inflow apices, and a plurality of cantilevered axial extensions, each axial extension being disposed between a pair of adjacent inflow apices; and a plurality of valve leaflets disposed within and coupled to the frame, each leaflet comprising a main body having an outflow edge portion and an inflow edge portion extending between a pair of adjacent inflow apices and having a movable portion coupled to a respective axial extension; wherein the movable portions of the leaflet inflow edge portions and the axial extensions are configured to move toward the longitudinal axis when the leaflets close under the back flow of blood and away from the longitudinal axis when the leaflets open under the forward flow of blood.

Example 28: The prosthetic heart valve of any example herein, particularly example 27, wherein each two adjacent inflow apices forms a circumferential gap therebetween, each axial extension being disposed in one of the gaps.

Example 29: The prosthetic heart valve of any example herein, particularly any one of examples 27-28, wherein each axial extension comprises a free end portion and a fixed end portion, the axial extension configured to bend inwardly into the frame when the leaflets close under the back flow of blood.

Example 30: The prosthetic heart valve of any example herein, particularly any one of examples 27-29, wherein each axial extension is disposed midway between its respective pair of adjacent inflow apices.

Example 31: The prosthetic heart valve of any example herein, particularly any one of examples 27-30, the frame further comprising an outer skirt mounted to the outer surface of the frame, the outer skirt coupled to one or more axial extensions.

Example 32: The prosthetic heart valve of any example herein, particularly example 31, wherein the outer skirt is coupled to the inflow edge of each leaflet.

Example 33: The prosthetic heart valve of any example herein, particularly any one of examples 31-32, wherein the outer skirt is configured to move with the axial extensions and/or leaflets coupled thereto.

Example 34: The prosthetic heart valve of any example herein, particularly any one of examples 27-33, wherein each axial extension extends from an axially extending support post of the frame.

Example 35: The prosthetic heart valve of any example herein, particularly example 34, wherein each axially extending support post of the frame is disposed between adjacent pairs of circumferentially disposed cells of the frame.

Example 36: The prosthetic heart valve of any example herein, particularly example 35, wherein the cells form the outflow and inflow apices of the frame.

Example 37: The prosthetic heart valve of any example herein, particularly any one of examples 35-36, wherein the cells are axially extending elliptical cells.

Example 38: The prosthetic heart valve of any example herein, particularly any one of examples 35-37, wherein each cell is formed by a row of upper struts that form the outflow end portion of the frame, and a row of lower struts that from the inflow end portion of the frame.

Example 39: The prosthetic heart valve of any example herein, particularly example 38, wherein the inflow edge portion of each leaflet comprises an apex edge portion coupled to a respective axial extension and an angled edge portion coupled to the lower struts that form the outflow end portion of the frame.

Example 40: The prosthetic heart valve of any example herein, particularly any one of examples 35-39, wherein each cell is an outer cell, each outer cell having an inner cell disposed within the outer perimeter of the outer cell.

Example 41: The prosthetic heart valve of any example herein, particularly example 40, further comprising an outer skirt coupled the inner cell disposed within the outer perimeter of the outer cell.

Example 42: The prosthetic heart valve of any example herein, particularly any one of examples 35-41, wherein one or more cells comprises a pair of axially aligned posts configured to be axially spaced when the frame is in a compressed or partially expanded configuration and to contact each other when the frame is in a fully expanded configuration.

Example 43: The prosthetic heart valve of any example herein, particularly example 42, wherein each pair of axially aligned posts comprises a first axial post extending into the cell from a respective outflow apex and a second axial post extending into the cell from a respective inflow apex.

Example 44: The prosthetic heart valve of any example herein, particularly any one of examples 42-43, wherein each pair of axially aligned posts is configured to prevent an outer skirt mounted to an outer surface of the frame from extending through an opening formed by the cell.

Example 45: The prosthetic heart valve of any example herein, particularly any one of examples 42-44, further comprising an actuator member extending through each pair of axially aligned posts.

Example 46: The prosthetic heart valve of any example herein, particularly example 45, wherein each actuator member further comprises a threaded rod extending through an inner bore of the posts.

Example 47: The prosthetic heart valve of any example herein, particularly example 46, wherein each rod is configured to rotate such that the posts move axially toward one another and radially expand the frame.

Example 48: The prosthetic heart valve of any example herein, particularly any one of examples 27-47, wherein each axial extension comprises at least one opening.

Example 49: The prosthetic heart valve of any example herein, particularly example 48, wherein the inflow edge portion of each leaflet is coupled to a respective axial extension via sutures extending through the at least one opening of the axial extension.

Example 50: The prosthetic heart valve of any example herein, particularly any one of examples 27-41, wherein the movable portion of the inflow edge portion of each leaflet is coupled to the frame only at a respective axial extension.

Example 51: The prosthetic heart valve of any example herein, particularly any one of examples 27-50, the frame further comprising one or more commissure clasps disposed between one or more adjacent pairs of outflow apices, each clasp comprises a first commissure arm, a second commissure arm, and an opening therebetween configured to receive a commissure formed by two adjacent leaflets.

Example 52: The prosthetic heart valve of any example herein, particularly example 51, wherein the first commissure arm has a first indentation and the second commissure arm has a second indentation, the first and second indentations configured to receive a fastener such that the first and second commissure arms and the fastener restrict axial movement of the commissure received therein.

Example 53: The prosthetic heart valve of any example herein, particularly example 52, wherein each fastener comprises a suture tightened around a pair of first and second commissure arms.

Example 54: The prosthetic heart valve of any example herein, particularly any one of examples 27-53, wherein the leaflet inflow edge portions and the axial extensions are configured to move laterally toward adjacent inflow apices when a force is applied to the axial extensions.

Example 55: The prosthetic heart valve of any example herein, particularly example 54, wherein a radial width of the axial extensions is greater than a circumferential width of the axial extensions.

Example 56: The prosthetic heart valve of any example herein, particularly any one of examples 54-55, wherein a radial width of a free end portion of the axial extensions is greater than a radial width of a fixed end portion of the axial extensions such that the axial extensions are configured to move toward the longitudinal axis of the frame.

Example 57: The prosthetic heart valve of any example herein, particularly any one of examples 54-55, wherein a radial width of a free end portion of the axial extensions is equal to a radial width of a fixed end portion of the axial extensions.

Example 58: The prosthetic heart valve of any example herein, particularly any one of examples 56-57, wherein a circumferential width of the free end portions is greater than a circumferential width of the fixed end portions.

Example 59: A prosthetic heart valve delivery assembly, the delivery assembly comprising: a delivery apparatus comprising a handle and a shaft having a proximal end portion coupled to the handle and distal end portion; and an expandable prosthetic heart valve coupled to the distal end portion of the shaft; wherein the prosthetic heart valve comprises a radially expandable frame comprising an outflow end, an inflow end, and a plurality of valve leaflets disposed within and coupled to the frame, each leaflet comprising a main body having an outflow edge portion and an inflow edge portion, wherein the leaflets are configured to move between an open state allowing blood to flow through the frame from the inflow end to the outflow end and a closed state in which the outflow edge portions coapt with each other and block the flow of blood through the frame from the outlet end to the inlet end, wherein the inflow edge portion of each leaflet includes a movable portion that can move radially inwardly when the leaflets move to the closed state to assist with the coaptation of the outflow edge portions of the leaflets and radially outwardly when the leaflets move to the open state.

Example 60: The delivery assembly of any example herein, particularly example 59, the frame of prosthetic heart valve further comprising a plurality of outflow and inflow apices and a plurality of axial extensions, each axial extension being disposed between a pair of adjacent inflow apices and configured to move radially inwardly when the leaflets move to the closed state to assist with the coaptation of the outflow edge portions of the leaflets and radially outwardly when the leaflets move to the open state.

Example 61: The delivery assembly of any example herein, particularly any one of examples 59-60, the prosthetic heart valve further comprising an outer skirt mounted to the outer surface of the frame, an inflow edge of the outer skirt being coupled to the inflow edge portions of the leaflets.

Example 62: The delivery assembly of any example herein, particularly any one of examples 59-61, the frame of the prosthetic heart valve further comprising a commissure support member disposed between one or more adjacent pairs of outflow apices and retaining a commissure formed by two adjacent leaflets.

Example 63: A prosthetic heart valve comprising: a radially expandable frame comprising an inflow end, an outflow end, a circumferentially extending row of cells, a plurality of axially extending first posts having first ends within the cells, a plurality of axially extending second posts having second ends within the cells, wherein each of the first posts is aligned with one of the second posts along a length of the frame to form a pair of first and second posts, and a plurality of actuator members configured to radially expand the frame from a radially compressed state to a radially expanded state; wherein when the frame is in the radially compressed state, the first and second ends are axially spaced from each other and when the frame is in the radially expanded state, the first and second ends contact each other to prevent overexpansion of the frame; and a plurality of leaflets disposed inside the frame and configured to regulate the flow of blood in one direction through the frame.

Example 64: The prosthetic heart valve of any example herein, particularly example 63, wherein each actuator member extends through a pair of first and second posts.

Example 65: The prosthetic heart valve of any example herein, particularly example 64, wherein the actuator members are rotatable relative to the first and second posts to produce radial expansion of the frame.

Example 66: The prosthetic heart valve of any example herein, particularly example 65, wherein each actuator member comprises external threads that engage internal threads of a respective second post.

Example 67: The prosthetic heart valve of any example herein, particularly example 64, wherein the actuators are slidable in an axial direction relative to the first and second posts.

Example 68: The prosthetic heart valve of any example herein, particularly any one of examples 63-67, wherein a first length of the first posts and a second length of the second posts are equal.

Example 69: The prosthetic heart valve of any example herein, particularly any one of examples 63-67, wherein one of the first post and the second post of each pair of first and second posts has a first length different than a second length of the other one of the first post and the second post.

Example 70: The prosthetic heart valve of any example herein, particularly any one of examples 63-69, wherein the first posts extend axially from the outflow end of the frame and the second posts extend axially from the inflow of the frame.

Example 71: The prosthetic heart valve of any example herein, particularly any one of examples 63-70, wherein each cell comprises an outer cell extending from the inflow end to the outflow end of the frame and an inner cell disposed within the outer cell.

Example 72: The prosthetic heart valve of any example herein, particularly example 71, wherein the first ends of the first posts and the second ends of the second posts are within the inner cell.

Example 73: The prosthetic heart valve of any example herein, particularly any one of examples 71-72, wherein the outer and inner cells form an axially-extending elliptical shape, each outer and inner cell having a respective inflow apex and an outflow apex.

Example 74: The prosthetic heart valve of any example herein, particularly example 73, wherein the first posts extend axially between the outflow apices of the outer and inner cells and the second posts extend axially between the inflow apices of the outer and inner cells.

Example 75: The prosthetic heart valve of any example herein, particularly any one of examples 63-74, the frame further comprising a plurality of cantilevered axial struts, each axial strut disposed between adjacent pairs of first and second posts.

Example 76: The prosthetic heart valve of any example herein, particularly example 75, wherein each axial strut is disposed midway between respective adjacent pairs of first and second posts.

Example 77: The prosthetic heart valve of any example herein, particularly any one of examples 75-76, wherein each axial strut is disposed between a pair of adjacent cells within the row of cells.

Example 78: The prosthetic heart valve of any example herein, particularly any one of examples 75-77, the frame further comprising a plurality of inflow apices at the inflow end of the frame, each axial strut disposed between a pair of adjacent inflow apices.

Example 79: The prosthetic heart valve of any example herein, particularly any one of examples 63-77, wherein each leaflet comprises an outflow edge portion and an inflow edge portion, the inflow edge portions including a movable portion that can move radially inwardly when the leaflets move to a closed state in which the outflow edge portions coapt with one another and radially outwardly when the leaflets move to an open state in which blood flow is allowed to flow through the frame from the inflow end to the outflow end.

Example 80: The prosthetic heart valve of any example herein, particularly example 79, when combined with any of examples 75-78, wherein each axial strut comprises a free end portion and a fixed end portion, and the movable portions of the leaflet inflow edge portions are connected to the free end of the axial struts, the axial struts being configured to bend inwardly into the frame when the leaflets close under the back flow of blood.

Example 81: The prosthetic heart valve of any example herein, particularly example 80, wherein the fixed end portions of the axial struts comprise a narrowed section in which the axial struts have increased flexibility to bend inwardly into the frame.

Example 82: The prosthetic heart valve of any example herein, particularly any one of examples 80-81, wherein the axial struts have a length such that the free end portions of the axial struts align with the inflow end of the frame when the frame is in the radially expanded state.

Example 83: The prosthetic heart valve of any example herein, particularly any one of examples 63-82, the frame further comprising a plurality of commissure support members, each support member disposed between adjacent pairs of first and second posts.

Example 84: The prosthetic heart valve of any example herein, particularly example 83, wherein each commissure support member is disposed midway between respective adjacent pairs of first and second posts.

Example 85: The prosthetic heart valve of any example herein, particularly any one of examples 83-84, wherein each commissure support member is disposed between a pair of adjacent cells within the row of cells.

Example 86: The prosthetic heart valve of any example herein, particularly any one of examples 83-85, wherein the commissure support members comprise a first commissure arm, a second commissure arm, and an opening therebetween configured to receive a leaflet commissure formed by two adjacent leaflets.

Example 87: The prosthetic heart valve of any example herein, particularly example 86, each commissure support member further comprising a flexible member wrapped around the first and second commissure arms between the leaflet commissure and the outflow end of the frame to secure the leaflet commissure within the commissure support member opening.

Example 88: The prosthetic heart valve of any example herein, particularly example 87, wherein the first and second commissure arms each comprise an indentation which receives the flexible member.

Example 89: The prosthetic heart valve of any example herein, particularly any one of examples 87-88, wherein the flexible member is a suture.

Example 90: The prosthetic heart valve of any example herein, particularly any one of examples 86-89, wherein each leaflet commissure comprises a first commissure tab of one adjacent leaflet wrapped around the first commissure arm and a second commissure tab of the other adjacent leaflet wrapped around the second commissure arm, wherein the first and second commissure tabs are sutured to one another inside the frame.

Example 91: The prosthetic heart valve of any example herein, particularly example 90, wherein a first end of the first commissure tab is adjacent to the body of its respective leaflet and a second end of the second commissure tab is adjacent to the body of its respective leaflet, wherein one or more sutures are stitched through the first and second commissure tabs and the bodies of the leaflets inside the frame to secure the leaflet commissures.

Example 92: A prosthetic heart valve comprising: a radially expandable frame comprising an outflow end portion, an inflow end portion, a plurality of outflow and inflow apices, and a plurality of cantilevered axial extensions, each axial extension being disposed between a pair of adjacent inflow apices; and a plurality of valve leaflets disposed within and coupled to the frame, each leaflet comprising a main body having an outflow edge portion and an inflow edge portion coupled to a respective axial extension; wherein the leaflet inflow edge portions and the axial extensions are configured to move laterally toward adjacent inflow apices when a force is applied to the axial extensions.

Example 93: The prosthetic heart valve of any example herein, particularly example 92, the frame further comprising an outer skirt mounted to the outer surface of the frame, the outer skirt coupled to one or more axial extensions.

Example 94: The prosthetic heart valve of any example herein, particularly example 93, wherein the outer skirt is coupled to the inflow edge of each leaflet.

Example 95: The prosthetic heart valve of any example herein, particularly any one of examples 93-94, wherein the outer skirt is configured to move laterally with the axial extensions and/or leaflets coupled thereto.

Example 96: The prosthetic heart valve of any example herein, particularly any one of examples 92-95, wherein a radial width of the axial extensions is greater than a circumferential width of the axial extensions.

Example 97: The prosthetic heart valve of any example herein, particularly any one of examples 92-96, wherein each axial extension comprises a free end portion and a fixed end portion.

Example 98: The prosthetic heart valve of any example herein, particularly example 97, wherein a radial width of the free end portions is greater than a radial width of the fixed end portions.

Example 99: The prosthetic heart valve of any example herein, particularly example 97, wherein a radial width of the free end portions is equal to a radial width of the fixed end portions.

Example 100: The prosthetic heart valve of any example herein, particularly any one of examples 97-99, wherein a circumferential width of the free end portions is greater than a circumferential width of the fixed end portions.

Example 101: The prosthetic heart valve of any example herein, particularly any one of examples 97-100, wherein the free end portions taper toward the fixed end portions.

Example 102: The prosthetic heart valve of any example herein, particularly any one of examples 97-101, wherein the free end portions of the axial extensions have a rounded shape and comprise an aperture.

Example 103: The prosthetic heart valve of any example herein, particularly example 102, wherein the aperture is configured to receive a suture therethrough.

Example 104: The prosthetic heart valve of any example herein, particularly any one of examples 92-103, wherein each axial extension is configured to compress axially in a direction toward of the outflow end portion of the frame.

Example 105: The prosthetic heart valve of any example herein, particularly any one of examples 92-104, wherein each axial extension is curved along its length.

Example 106: The prosthetic heart valve of any example herein, particularly any one of examples 92-105, wherein each axial extension is asymmetrical along a central longitudinal axis bisecting the axial extension.

Example 107: The prosthetic heart valve of any example herein, particularly any one of examples 92-106, wherein each axial extension is disposed midway between its respective pair of adjacent inflow apices.

Example 108: The prosthetic heart valve of any example herein, particularly any one of examples 92-107, wherein each axial extension extends from an axially extending support post of the frame.

Example 109: The prosthetic heart valve of any example herein, particularly any one of examples 92-108, wherein each axial extension of the frame is disposed between adjacent pairs of circumferentially disposed cells of the frame.

Example 110: The prosthetic heart valve of any example herein, particularly any one of examples 92-109, the frame further comprising a central longitudinal axis extending from the inflow end portion to the outflow end portion, wherein the inflow edge portion of each leaflet has a moveable portion coupled to a respective axial extension, wherein the movable portions of the leaflet inflow edge portions and the axial extensions are configured to move toward the longitudinal axis when the leaflets close under the back flow of blood and away from the longitudinal axis when the leaflets open under the forward flow of blood.

Examples 111: The prosthetic heart valve of any example herein, particularly any one of examples 92-110, the frame further comprising a central longitudinal axis extending from the inflow end portion to the outflow end portion, wherein each axial extension is configured to resist radial inward movement toward the longitudinal axis of the frame when the leaflets close under the back flow of blood and away from the longitudinal axis when the leaflets open under the forward flow of blood.

Example 112: A prosthetic heart valve comprising: a radially expandable frame comprising an inflow end, an outflow end, and a plurality of struts arranged to form a circumferentially extending row of struts forming the inflow end, wherein one or more selected struts have at least one opening extending therethrough; and a plurality of leaflets disposed inside the frame and configured to regulate the flow of blood in one direction through the frame, each leaflet comprising an outflow edge portion and an inflow edge portion; wherein the inflow edge portions of the leaflets are coupled to the selected struts of the frame with sutures extending through the openings.

Example 113: The prosthetic heart valve of any example herein, particularly example 112, wherein the selected struts are arranged in pairs of adjacent selected struts.

Example 114: The prosthetic heart valve of any example herein, particularly example 113, wherein for each pair of adjacent selected struts, one of the struts is coupled to one of the leaflets and the other strut is coupled to an adjacent leaflet.

Example 115: The prosthetic heart valve of any example herein, particularly any one of examples 113-114, wherein the row of struts comprises a pair of adjacent struts lacking an opening disposed between each pair of adjacent selected struts.

Example 116: The prosthetic heart valve of any example herein, particularly any one of examples 112-115, wherein each selected strut has an opening extending radially therethrough.

Example 117: The prosthetic heart valve of any example herein, particularly any one of examples 112-116, wherein the selected struts have a plurality of openings extending radially therethrough.

Example 118: The prosthetic heart valve of any example herein, particularly any one of examples 110-117, wherein each selected strut comprises an inflow section, a midsection, and an outflow section, wherein the openings radially extend through the midsection of the struts.

Example 119: The prosthetic heart valve of any example herein, particularly example 118, wherein the midsection of the selected struts has a rounded edge.

Example 120: The prosthetic heart valve of any example herein, particularly example 118, wherein the midsection of the selected struts comprises a pair of parallel rounded edges.

Example 121: The prosthetic heart valve of any example herein, particularly any one of examples 118-120, wherein the midsection of the selected struts has a circumferential width greater than a circumferential width of the inflow and outflow sections of the selected struts.

Example 122: The prosthetic heart valve of any example herein, particularly any one of examples 118-121, wherein the inflow section and the outflow section of the selected struts are configured to bend relative to the midsection of the selected struts during radial expansion of the frame.

Example 123: The prosthetic heart valve of any example herein, particularly any one of examples 118-122, the frame further comprising a plurality of axially extending posts, wherein each of the posts comprises an indentation configured to receive the midsection of an adjacent selected strut when the frame is in a radially compressed state.

Example 124: The prosthetic heart valve of any example herein, particularly example 123, wherein the indentation of each post is formed in a longitudinal edge of the post.

Example 125: The prosthetic heart valve of any example herein, particularly any one of examples 112-124, wherein the sutures form whip stitches that extend through the openings and the inflow edge portions of the leaflets.

Example 126: The prosthetic heart valve of any example herein, particularly any one of examples 112-125, wherein the inflow edge portion of each leaflet comprises two angled edge portions, each of which is coupled to an adjacent selected strut via a suture extending through the opening of the selected strut.

Example 127: The prosthetic heart valve of any example herein, particularly any one of examples 112-126, wherein each opening is circular in shape.

Example 128: The prosthetic heart valve of any example herein, particularly any one of examples 112-126, wherein each opening is rectangular in shape.

Example 129: The prosthetic heart valve of any example herein, particularly any one of examples 113-128, the frame further comprising a plurality of cantilevered axial extensions, each of which is disposed between a pair of adjacent selected struts.

Example 130: The prosthetic heart valve of any example herein, particularly example 129, wherein each axial extension disposed between a pair of adjacent selected struts lacks an opening.

Example 131: The prosthetic heart valve of any example herein, particularly any one of examples 115-130, the frame further comprising a plurality of cantilevered axial extensions, each of which is disposed between a pair of adjacent struts lacking an opening.

Example 132: The prosthetic heart valve of any example herein, particularly example 131, wherein each axial extension disposed between a pair of adjacent struts lacking an opening, has an opening extending radially therethrough.

Example 133: The prosthetic heart valve of any example herein, particularly any one of examples 112-128, the frame further comprising a plurality of cantilevered axial extensions, each of which extends from a junction between two adjacent struts of the row of struts.

Example 134: The prosthetic heart valve of any example herein, particularly example 133, wherein at least one axial extension has an opening extending radially therethrough and at least one axial extension lacks an opening.

Example 135: The prosthetic heart valve of any example herein, particularly example 134, wherein each axial extension has a free end and a fixed end coupled to a respective junction between two adjacent struts.

Example 136: The prosthetic heart valve of any example herein, particularly example 135, wherein the free ends of the axial extensions having an opening have a first diameter and the free ends of the axial extensions lacking an opening have a second diameter, wherein the first diameter is greater than the second diameter.

Example 137: The prosthetic heart valve of any example herein, particularly any one of examples 133-136, wherein at least half of the axial extensions have an opening extending radially therethrough.

Example 138: The prosthetic heart valve of any example herein, particularly any one of examples 133-137, wherein at least half of the axial extensions lack an opening.

Example 139: The prosthetic heart valve of any example herein, particularly any one of examples 112-138, wherein the frame comprises three pairs of adjacent struts lacking an opening and three pairs of adjacent selected struts arranged in an alternating pattern circumferentially around the frame.

Example 140: The prosthetic heart valve of any example herein, particularly any one of examples 112-139, the frame further comprising a plurality of outflow apices and inflow apices.

Example 141: The prosthetic heart valve of any example herein, particularly example 140, when combined with any of examples 129-138, the frame further comprising a commissure support member disposed between one or more adjacent pairs of outflow apices.

Example 142: The prosthetic heart valve of any example herein, particularly example 141, wherein at least one axial extension is aligned with a respective commissure support member.

Example 143: The prosthetic heart valve of any example herein, particularly any one of examples 129-142, wherein at least one axial extension is axially aligned with a leaflet commissure formed by two adjacent leaflets and mounted to the frame.

Example 144: The prosthetic heart valve of any example herein, particularly any one of examples 140-143, wherein a circumferential width of each inflow apex is less than a circumferential width of each outflow apex.

Example 145: The prosthetic heart valve of any example herein, particularly any one of examples 140-144, wherein the inflow edge portion of each leaflet extends between a pair of adjacent inflow apices.

Example 146: The prosthetic heart valve of any example herein, particularly any one of examples 112-145, the frame further comprising a central longitudinal axis extending from the inflow end to the outflow end, wherein the inflow edge portion of each leaflet has a movable portion configured to move toward the longitudinal axis when the leaflets close under the back flow of blood and away from the longitudinal axis when the leaflets open under the forward flow of blood.

Example 147: The prosthetic heart valve of any example herein, particularly example 146, when combined with any of examples 129-138, wherein the movable portions of the leaflet inflow edge portions are coupled to respective axial extensions.

Example 148: The prosthetic heart valve of any example herein, particularly example 147, wherein the movable portions of the leaflet inflow edge portions and the axial extensions are configured to move toward the longitudinal axis when the leaflets close under the back flow of blood and away from the longitudinal axis when the leaflets open under the forward flow of blood.

Example 149: The prosthetic heart valve of any example herein, particularly any one of examples 147-148, wherein the movable portions of the leaflet inflow edge portions and the axial extensions are configured to move in a circumferential direction when a force is applied to the axial extensions.

Example 150: The prosthetic heart valve of any example herein, particularly any one of examples 147-149, wherein the inflow edge portion of each leaflet comprises an apex edge portion coupled to a respective axial extension and two angled edge portions on opposite side of the apex portion coupled to selected struts of the frame.

Example 151: The prosthetic heart valve of any example herein, particularly any one of examples 112-150, further comprising an outer skirt mounted to an outer surface of the frame.

Example 152: The prosthetic heart valve of any example herein, particularly example 151, when combined with any of examples 129-138, wherein at least one axial extension is configured to limit radially inwardly movement of the outer skirt into the frame.

Example 153: The prosthetic heart valve of any example herein, particularly any one of examples 151-152, wherein the outer skirt is connected to the frame with sutures.

Example 154: The prosthetic heart valve of any example herein, particularly example 153, wherein the sutures connecting the outer skirt to the frame extend through the openings of the selected struts.

Example 155: The prosthetic heart valve of any example herein, particularly any one of examples 112-156, further comprising a connecting skirt sutured to the inflow edge portions of the leaflets, wherein the sutures that extend through the openings in the selected struts extend through the connecting skirt to couple the inflow edge portions of the leaflets to the selected struts.

Example 156: The prosthetic heart valve of any example herein, particularly example 155, wherein the sutures that extend through the openings of the selected struts also form whip stitches that extend around the selected struts and through the connecting skirt.

Example 157: A prosthetic heart valve comprising: a radially expandable frame comprising an outflow end, an inflow end, and a central longitudinal axis extending from the inflow end portion to the outflow end portion; and a plurality of valve leaflets disposed within and coupled to the frame, each leaflet comprising a main body having an outflow edge and an inflow edge, and two commissure tabs on opposite sides of the main body, wherein the leaflets are configured to move between an opened state allowing blood to flow through the frame from the inflow end to the outflow end and a closed state in which the outflow edges coapt with each other and block the flow of blood through the frame from the outlet end to the inlet end; wherein each commissure tab is paired with a commissure tab of an adjacent leaflet to form a plurality of commissures coupled to respective commissure support portions of the frame, wherein the leaflets define an outflow channel that is tapered toward the outflow edges of the leaflets when the leaflets are in the opened state.

Example 158: The prosthetic heart valve of any example herein, particularly example 157, wherein the outflow channel of the leaflets is tapered 360-degrees around the leaflets.

Example 159: The prosthetic heart valve of any example herein, particularly any one of examples 157-158, wherein the outflow channel of the leaflets tapers from inflow ends of the commissures to the outflow edges of the leaflets.

Example 160: The prosthetic heart valve of any example herein, particularly any one of examples 157-159, wherein when the leaflets are in the opened state the outflow edges are tensioned.

Example 161: The prosthetic heart valve of any example herein, particularly example 160, wherein when the leaflets are in the opened state, tension across the leaflets in a plane intersecting the inflow ends of the commissures is less than the tension across the outflow edges.

Example 162: The prosthetic heart valve of any example herein, particularly example 160, wherein when the leaflets are in the opened state there is no tension across the leaflets in a plane intersection the inflow ends of the commissures.

Example 163: The prosthetic heart valve of any example herein, particularly any one of examples 161-162, wherein tension across the leaflets progressively increases from the plane intersecting the inflow ends of the commissures toward the outflow edges of the leaflets.

Example 164: The prosthetic heart valve of any example herein, particularly any one of examples 157-163, wherein the outflow edges of the leaflets are offset inwardly from an inner surface of the frame when the leaflets are in the opened state.

Example 165: The prosthetic heart valve of any example herein, particularly any one of examples 157-164, wherein when the leaflets are in the opened state, a first cross-sectional area formed by the inflow edges of the leaflets is greater than a second cross-sectional area formed by the outflow edges of the leaflets, the first and second cross-sectional areas being perpendicular to the longitudinal axis of the frame.

Example 166: The prosthetic heart valve of any example herein, particularly any one of examples 157-165, wherein a cross-sectional area of the outflow channel defined by the leaflets decreases from the inflow ends of the commissures to the outflow edges.

Example 167: The prosthetic heart valve of any example herein, particularly any one of examples 157-166, wherein the frame is cylindrical in an expanded state.

Example 168: The prosthetic heart valve of any example herein, particularly any one of examples 157-167, wherein an inner edge of each commissure is angled radially inwardly toward the longitudinal axis of the frame.

Example 169: The prosthetic heart valve of any example herein, particularly example 168, wherein the inner edge of each commissure extends from an inflow end of the commissure to an outflow edge of the leaflets at an angle greater than zero relative to an inner surface of the frame.

Example 170: The prosthetic heart valve of any example herein, particularly any one of examples 157-169, wherein for each leaflet, a width of the leaflet between the commissure tabs at sub-commissure portions of the leaflet is greater than a width of the leaflet between the commissure tabs at the outflow edge of the leaflet.

Example 171: The prosthetic heart valve of any example herein, particularly any one of examples 157-170, wherein for each leaflet, the commissure tabs are angled relative to the outflow edge of the leaflet.

Example 172: The prosthetic heart valve of any example herein, particularly any one of examples 157-171, wherein each pair of commissure tabs forming a leaflet commissure are sutured to one another along a stitch line angled inwardly toward a longitudinal axis of each respective leaflet.

Example 173: The prosthetic heart valve of any example herein, particularly any one of examples 157-172, wherein for each leaflet, the commissure tabs comprise respective lower tabs and respective upper tabs extending from the lower tabs, wherein each upper tab is folded against and sutured to a corresponding lower tab along a stitch line.

Example 174: The prosthetic heart valve of any example herein, particularly example 173, wherein each stitch line is angled inwardly toward a longitudinal axis of the leaflet.

Example 175: The prosthetic heart valve of any example herein, particularly any one of examples 173-174, wherein for each leaflet, the stitch line of each commissure tab is angled relative to the outflow edge of the leaflet.

Example 176: The prosthetic heart valve of any example herein, particularly any one of examples 157-175, wherein each commissure support portion comprises a first post, a second post, and an opening therebetween, wherein each leaflet commissure extends through an opening of an adjacent commissure support portion.

Example 177: A prosthetic heart valve comprising: a radially expandable frame comprising an outflow end and an inflow end; and a plurality of valve leaflets disposed within and coupled to the frame, each leaflet comprising a main body having an outflow edge and an inflow edge, and two commissure tabs on opposite sides of the main body, wherein the leaflets are configured to move between an opened state allowing blood to flow through the frame from the inflow end to the outflow end and a closed state in which the outflow edges coapt with each other and block the flow of blood through the frame from the outlet end to the inlet end; wherein each commissure tab is paired with a commissure tab of an adjacent leaflet to form a plurality of commissures coupled to respective commissure support portions of the frame and having inflow ends and outflow ends, wherein the leaflets are tensioned across the outflow edges of the leaflets when the leaflets are in the opened state.

Example 178: The prosthetic heart valve of any example herein, particularly example 177, wherein tension across the leaflets progressively decreases from the outflow edges to the inflow ends of the commissures.

Example 179: The prosthetic heart valve of any example herein, particularly any one of examples 177-178, wherein when the outflow edges are tensioned, there is no tension at the inflow ends of the commissures.

Example 180: The prosthetic heart valve of any example herein, particularly any one of examples 177-178, wherein the leaflets are tensioned from the inflow ends of the commissures to the outflow edges of the leaflets.

Example 181: The prosthetic heart valve of any example herein, particularly example 180, wherein tension across the leaflets at the outflow edges is greater than tension across the leaflets at the inflow ends of the commissures.

Example 182: The prosthetic heart valve of any example herein, particularly any one of examples 180-181, wherein tension across the leaflets between the outflow edges and the inflow ends of the commissures is less than the tension at the outflow edges and greater than the tension at the inflow ends.

Example 183: The prosthetic heart valve of any example herein, particularly any one of examples 180-182, wherein tension across the leaflets between the outflow edges of the leaflets and the inflow ends of the commissures is greater than the tension across the leaflets between the inflow ends of the commissures and the inflow edges of the leaflets.

Example 184: The prosthetic heart valve of any example herein, particularly any one of examples 177-183, wherein tension across the leaflets defines a tapered outflow channel when the leaflets are in the opened state.

Example 185: The prosthetic heart valve of any example herein, particularly any one of examples 177-184, wherein when the leaflets are tensioned, a radial gap extends between the outflow edges of the leaflets and an inner surface of the frame.

Example 186: The prosthetic heart valve of any example herein, particularly any one of examples 177-185, wherein when the leaflets are tensioned, the outflow edges are at an angle greater than zero relative to an inner surface of the frame.

Example 187: The prosthetic heart valve of any example herein, particularly any one of examples 177-186, wherein the tension across the leaflets extends 360 degrees around the plurality of leaflets.

Example 188: The prosthetic heart valve of any example herein, particularly any one of examples 177-187, wherein an inner diameter of the leaflets at the outflow edges is less than an inner diameter of the leaflets at the inflow ends of the commissures.

Example 189: A leaflet for a prosthetic heart valve comprising: a main body comprising an inflow edge, an outflow edge, a longitudinal axis, and a pair of opposing commissure tabs, each commissure tab having an inflow end and an outflow end, and extending from a respective side of the main body at an angle greater than zero relative to the longitudinal axis of the main body.

Example 190: The leaflet of any example herein, particularly example 189, wherein a width of the main body between the commissure tabs at the inflow ends is greater than a width of the main body between the commissure tabs at the outflow ends.

Example 191: The leaflet of any example herein, particularly any one of examples 189-190, wherein a width of the main body between the commissure tabs progressively decreases from the inflow ends to the outflow ends.

Example 192: The leaflet of any example herein, particularly any one of examples 190-191, wherein a length of the outflow edge is equal to the width of the main body between the commissure tabs at the outflow ends.

Example 193: The leaflet of any example herein, particularly any one of examples 189-192, wherein each commissure tab comprises a lower tab and an upper tab folded against and sutured to the lower tab along a stitch line.

Example 194: The leaflet of any example herein, particularly example 193, wherein each stitch line is angled inwardly toward the longitudinal axis of the main body.

Example 195: The leaflet of any example herein, particularly any one of examples 193-194, wherein each stitch line is parallel to its respective commissure tab.

Example 196: The leaflet of any example herein, particularly any one of examples 193-195, wherein the stitch line of each commissure tab extends from the inflow end to the outflow end of the commissure tab.

Example 197: The leaflet of any example herein, particularly any one of examples 193-195, wherein a distance between the stitch lines of the commissure tabs at the inflow ends is greater than a distance between the stitch lines at the outflow ends.

Example 198: The leaflet of any example herein, particularly any one of examples 193-197, wherein a length of the outflow edge is less than the distance between the stitch lines at the inflow ends of the commissure tabs.

Example 199: The leaflet of any example herein, particularly any one of examples 193-198, wherein each upper tab is folded over a fold line which is perpendicular to the stitch line of its respective commissure tab.

Example 200: The leaflet of any example herein, particularly any one of examples 193-199, wherein each upper tab forms an inner edge of its respective commissure, the inner edge extending from the inflow ends the commissure tab to the outflow edge of the leaflet.

Example 201: The leaflet of any example herein, particularly any one of examples 189-200, wherein each commissure tab is angled relative to the outflow edge.

Example 202: The leaflet of any example herein, particularly any one of examples 189-201, wherein the inflow edge comprises axially extending sub-commissure edges that are parallel to the longitudinal axis, each commissure tab being angled relative to an adjacent sub-commissure edge.

Example 203: The leaflet of any example herein, particularly example 202, wherein each commissure tab extends from an adjacent sub-commissure edge to the outflow edge.

Example 204: A leaflet assembly comprising a plurality of leaflets of any example herein, particularly any one of examples 189-203, wherein each commissure tab is paired with a commissure tab of an adjacent leaflet to form a plurality of leaflet commissures.

Example 205: A method for assembling a prosthetic heart valve comprising: positioning within a radially expandable frame a leaflet assembly comprising a plurality of leaflets, each leaflet having an inflow edge, an outflow edge, and a pair of opposing commissure tabs, each commissure tab being paired with a commissure tab of an adjacent leaflet to form a plurality of leaflet commissures having inflow ends and outflow ends, wherein the frame comprises a plurality of commissure support portions; stretching each leaflet between its respective commissure tabs and along the outflow edge to position each leaflet commissure adjacent a commissure support portion of the frame; and coupling each commissure to its respective commissure support portion of the frame, wherein the leaflets of the leaflet assembly are configured to move between an opened state allowing blood to flow through the frame from the inflow end to the outflow end and a closed state in which the outflow edges coapt with each other and block the flow of blood through the frame from the outlet end to the inlet end.

Example 206: The method of any example herein, particularly example 205, wherein preceding positioning the leaflet assembly within the frame, the method further comprises: suturing the paired commissure tabs of adjacent leaflets to one another along a stitch line to form a respective commissure, each stitch line being angled inwardly toward a longitudinal axis of the leaflet assembly and frame.

Example 207: The method of any example herein, particularly example 206, wherein coupling each commissure to its respective commissure support portion of the frame further comprises coupling each commissure to its respective commissure support portion with the stitch line of the commissure extending in an axial direction and parallel to a longitudinal axis of the frame.

Example 208: The method of any example herein, particularly any one of examples 205-207, wherein preceding stretching each leaflet, for each leaflet, a length of the outflow edge is less than a distance between its respective commissures at the inflow ends.

Example 209: The method of any example herein, particularly example 208, wherein following stretching each leaflet, for each leaflet, the length of the outflow edge is equal to the distance between its respective commissures at the inflow ends.

Example 210: The method of any example herein, particularly any one of examples 205-209, wherein coupling each commissure to its respective commissure support portion comprises suturing each commissure to its respective commissure support portion.

Example 211: The method of any example herein, particularly any one of examples 205-210, wherein when the leaflets are in the opened state, the outflow edges are tensioned and radially offset from an inner surface of the frame.

Example 212: The method of any example herein, particularly example 211, wherein the leaflets are tensioned 360 degrees around the leaflet assembly.

Example 213: The method of any example herein, particularly any one of examples 211-212, wherein tension across the leaflet assembly defines an outflow channel that tapers toward the outflow edges of the leaflets.

Example 214: A prosthetic heart valve comprising: a radially expandable and compressible frame comprising an outflow end portion, an inflow end portion having a plurality of inflow apices, and a plurality of cantilevered axial extensions, each axial extension having a fixed end and free end disposed between a pair of adjacent inflow apices; and a plurality of valve leaflets disposed within and coupled to the frame, each leaflet comprising a main body having an outflow edge portion and an inflow edge portion coupled to a respective axial extension; wherein the leaflet inflow edge portions are secured to the free ends of the axial extensions.

Example 215: The prosthetic heart valve of any example herein, particularly example 214, wherein the free ends of the axial extensions comprise two or more outwardly extending arms sharing a common junction.

Example 216: The prosthetic heart valve of any example herein, particularly example 215, wherein the arms of the free ends are arranged in a V-shape.

Example 217: The prosthetic heart valve of any example herein, particularly example 215, wherein the arms of the free ends are arranged in a U-shape.

Example 218: The prosthetic heart valve of any example herein, particularly example 215, wherein the arms of the free ends are arranged in a X-shape.

Example 219: The prosthetic heart valve of any example herein, particularly any one of examples 215-218, wherein at least one arm comprises a projection along its surface.

Example 220: The prosthetic heart valve of any example herein, particularly any one of examples 215-219, wherein each arm is configured to deflect toward an adjacent arm when a force is applied to the free ends of the axial extensions.

Example 221: The prosthetic heart valve of any example herein, particularly any one of examples 215-220, wherein the leaflet inflow edge portions are secured to the arms of the free ends of the axial extensions by a suture extending through the leaflet edge portions and around the arms.

Example 222: The prosthetic heart valve of any example herein, particularly example 221, when dependent on claim 219, wherein a projection of at least one arm is configured to limit movement of the suture along the surface of the arm.

Example 223: The prosthetic heart valve of any example herein, particularly example 214, wherein the free ends of the axial extensions define a compressible eyelet that is sized and shaped to receive a suture therethrough.

Example 224: The prosthetic heart valve of any example herein, particularly example 223, wherein the eyelet is U-shaped.

Example 225: The prosthetic heart valve of any example herein, particularly example 224, wherein a portion of the eyelet is discontinuous and defines an open segment along the eyelet.

Example 226: The prosthetic heart valve of any example herein, particularly example 223, wherein the eyelet is elliptical in shape.

Example 227: The prosthetic heart valve of any example herein, particularly any one of examples 223-226, wherein the eyelets comprise a pair of lateral portions configured to move toward one another when a force is applied to the free ends of the axial extensions.

Example 228: The prosthetic heart valve of any example herein, particularly example 214, wherein the free ends of the axial extensions comprise a longitudinal edge and at least one cutout along the longitudinal edge.

Example 229: The prosthetic heart valve of any example herein, particularly example 214, wherein the free ends of the axial extensions comprise pairs of longitudinal edges and a plurality of cutouts along the longitudinal edges.

Example 230: The prosthetic heart valve of any example herein, particularly any one of examples 228-229, wherein the cutouts are sized and shaped to receive a suture.

Example 231: The prosthetic heart valve of any example herein, particularly any one of examples 214-230, wherein the axial extensions taper radially inwardly toward a longitudinal axis of the frame.

Example 232: The prosthetic heart valve of any example herein, particularly example 231, wherein the axial extensions are configured move radially outwardly from a longitudinal axis of the frame as the frame is radially compressed.

Example 233: A prosthetic heart valve comprising: a radially expandable frame comprising an inflow end, an outflow end, a plurality of axially extending first posts, and a plurality of axially extending second posts, wherein selected pairs of axially aligned first and second posts form a first set of selected posts and other selected pairs of axially aligned first and second posts form a second set of selected posts; the frame further comprising: a first set of nuts coupled to the second posts of the first set of selected posts and a second set of nuts coupled to the second posts of the second set of selected posts, wherein the first set of nuts differ in at least one dimension from the second set of nuts; a plurality of first actuator members extending through the first set of selected posts and the first set of nuts, and a plurality of second actuator members extending through the second set of selected posts and the second set of nuts, wherein the first actuator members are configured to rotate in a first direction and the second actuator members are configured to rotate in a second direction, the first and second actuator members being configured to radially expand the frame from a radially compressed state to a radially expanded state; and a plurality of leaflets disposed inside the frame and configured to regulate the flow of blood in one direction through the frame.

Example 234: The prosthetic heart valve of any example herein, particularly example 233, wherein each second post of the first set of selected posts includes a window configured to receive a respective nut of the first set of nuts and each second post of the second set of selected posts includes a window configured to receive a respective nut of the second set of nuts.

Example 235: The prosthetic heart valve of any example herein, particularly any one of examples 233-234, wherein the first set of nuts has a first axial length and the second set of nuts has a second axial length less than the first axial length.

Example 236: The prosthetic heart valve of any example herein, particularly any one of examples 233-235, wherein the first set of nuts has a first width and the second set of nuts has a second width less than the first width of the first set of nuts.

Example 237: The prosthetic heart valve of any example herein, particularly any one of examples 233-236, wherein the first set of selected posts and the second set of selected posts are arranged circumferentially around the frame in an alternating pattern.

Example 238: The prosthetic heart valve of any example herein, particularly any one of examples 233-237, wherein the first set of nuts and the second set of nuts are arranged circumferentially around the frame in an alternating pattern.

Example 239: The prosthetic heart valve of any example herein, particularly any one of examples 233-238, wherein the first and second sets of nuts comprise a first radiopaque material and the second posts comprise a second radiopaque material different from the first radiopaque material of first and second sets of nuts.

Example 240: The prosthetic heart valve of any example herein, particularly any one of examples 233-239, wherein at least one pair of first and second posts lack a respective nut and actuator member extending therethrough.

Example 241: The prosthetic heart valve of any example herein, particularly any one of examples 233-240, further comprising at least one additional set of nuts that differs in at least one dimension from the first and second sets of nuts.

Example 242: A prosthetic heart valve comprising: a radially expandable frame comprising an inflow end, an outflow end, and a plurality of axially extending posts, at least one post including an inner bore extending therethrough and an aperture extending from an external surface of the frame to the inner bore of the post; and a plurality of leaflets disposed inside the frame and configured to regulate the flow of blood in one direction through the frame.

Example 243: The prosthetic heart valve of any example herein, particularly example 242, wherein at least one post includes a plurality of apertures extending from an external surface of the frame to the inner bore of the post.

Example 244: The prosthetic heart valve of any example herein, particularly example 243, wherein the apertures are axially spaced from one another.

Example 245: The prosthetic heart valve of any example herein, particularly any one of examples 242-244, wherein at least one aperture extends from an inner surface of the frame to the inner bore of the post.

Example 246: The prosthetic heart valve of any example herein, particularly any one of examples 242-245, wherein at least one aperture extends from an outer surface of the frame to the inner bore of the post.

Example 247: The prosthetic heart valve of any example herein, particularly any one of examples 242-246, wherein one or more posts comprise at least one aperture extending from an inner surface of the frame to the inner bore of the post and at least one aperture extending from an outer surface of the frame to the inner bore of the post.

Example 248: The prosthetic heart valve of any example herein, particularly any one of examples 242-247, further comprising a plurality of actuator members extending through the inner bores of the posts.

Example 249: The prosthetic heart valve of any example herein, particularly any one of examples 242-248, wherein each axially extending post extends from a respective outflow apex of the frame.

Example 250: The prosthetic heart valve of any example herein, particularly any one of examples 242-249, wherein each axially extending post extends from a respective inflow apex of the frame.

Example 251: A prosthetic heart valve of any example herein, particularly any one of examples 1-250, wherein the prosthetic heart valve is sterilized.

Example 252: A prosthetic heart valve comprising: a radially expandable and compressible frame comprising an outflow end portion, an inflow end portion having a plurality of inflow apices, and a plurality of cantilevered axial extensions, each axial extension having a fixed end and free end disposed between a pair of adjacent inflow apices;

and a plurality of valve leaflets disposed within and coupled to the frame, each leaflet comprising a main body having an outflow edge portion and an inflow edge portion coupled to a respective axial extension; wherein the leaflet inflow edge portions are secured to the free ends of the axial extensions.

Example 253: The prosthetic heart valve of any example herein, particularly example 252, wherein the leaflet inflow edge portions and the free ends of the axial extensions are configured to move radially inwardly when the leaflets close under the back flow of blood and radially outwardly when the leaflets open under the forward flow of blood.

Example 254: The prosthetic heart valve of any example herein, particularly any one of examples 252-253, wherein the leaflet inflow edge portions and the free ends of the axial extensions are configured to move laterally toward adjacent inflow apices when a force is applied to the axial extensions.

Example 255: The prosthetic heart valve of any example herein, particularly any one of examples 252-254, the frame further comprising a central longitudinal axis extending from the inflow end portion to the outflow end portion, wherein the axial extensions are configured to resist radial movement toward the longitudinal axis of the frame when the leaflets close under the back flow of blood and away from the longitudinal axis when the leaflets open under the forward flow of blood.

Example 256: The prosthetic heart valve of any example herein, particularly any one of examples 252-255, the frame further comprising an outer skirt mounted to an outer surface of the frame, the outer skirt coupled to one or more axial extensions.

Example 257: The prosthetic heart valve of any example herein, particularly any one of examples 252-256, wherein the free ends of the axial extensions comprise an aperture.

Example 258: The prosthetic heart valve of any example herein, particularly any one of examples 252-257, wherein the free ends of the axial extensions are configured to compress in a circumferential direction of the frame when the frame is radially compressed.

Example 259: The prosthetic heart valve of any example herein, particularly any one of examples 252-258, the frame further comprising one or more commissure clasps, each clasp comprising a first commissure arm having a first indentation, a second commissure arm having a second indentation, and an opening therebetween configured to receive a commissure formed by two adjacent leaflets, wherein the first and second indentations are configured to receive a fastener such that the first and second commissure arms and the fastener restrict axial movement of the commissure received therein.

Example 260: A prosthetic heart valve comprising: a radially expandable frame comprising an inflow end, an outflow end, and a plurality of struts arranged to form a circumferentially extending row of struts forming the inflow end, wherein one or more selected struts have at least one opening extending therethrough; and a plurality of leaflets disposed inside the frame and configured to regulate the flow of blood in one direction through the frame, each leaflet comprising an outflow edge portion and an inflow edge portion; wherein the inflow edge portions of the leaflets are coupled to the selected struts of the frame with sutures extending through the openings.

Example 261: The prosthetic heart valve of any example herein, particularly example 260, wherein the selected struts are arranged in pairs of adjacent selected struts, wherein for a pair of adjacent selected struts, one of the selected struts is coupled to one of the leaflets and the other selected strut is coupled to an adjacent leaflet.

Example 262: The prosthetic heart valve of any example herein, particularly any one of examples 260-261, wherein each selected strut has one or more openings extending radially therethrough.

Example 263: The prosthetic heart valve of any example herein, particularly any one of examples 260-262, wherein each selected strut comprises an inflow section, a midsection, and an outflow section, wherein the openings radially extend through the midsection of the struts.

Example 264: The prosthetic heart valve of any example herein, particularly example 263, wherein the midsection of the selected struts has a circumferential width greater than a circumferential width of the inflow and outflow sections of the selected struts.

Example 265: The prosthetic heart valve of any example herein, particularly example 264, the frame further comprising a plurality of axially extending posts, wherein each of the posts comprises an indentation configured to receive the midsection of an adjacent selected strut when the frame is in a radially compressed state.

Example 266: The prosthetic heart valve of any example herein, particularly any one of examples 260-265, further comprising an outer skirt mounted to an outer surface of the frame and connected to the frame with sutures, wherein the sutures extend through the openings of the selected struts.

Example 267: The prosthetic heart valve of any example herein, particularly any one of examples 260-266, the frame further comprising a plurality of axially extending first posts and a plurality of axially extending second posts, wherein selected pairs of axially aligned first and second posts form a first set of selected posts and other selected pairs of axially aligned first and second posts form a second set of selected posts; wherein the frame further comprises: a first set of nuts coupled to the second posts of the first set of selected posts and a second set of nuts coupled to the second posts of the second set of selected posts, wherein the first set of nuts differ in at least one dimension from the second set of nuts; and a plurality of first actuator members extending through the first set of selected posts and the first set of nuts, and a plurality of second actuator members extending through the second set of selected posts and the second set of nuts, wherein the first actuator members are configured to rotate in a first direction and the second actuator members are configured to rotate in a second direction, the first and second actuator members being configured to radially expand the frame from a radially compressed state to a radially expanded state.

Example 268: The prosthetic heart valve of any example herein, particularly any one of examples 260-267, the frame further comprising a plurality of axially extending posts, at least one post comprising an inner bore extending therethrough and an aperture extending from an external surface of the frame to the inner bore of the post.

Example 269: A prosthetic heart valve comprising: a radially expandable frame comprising an outflow end and an inflow end; and a plurality of valve leaflets disposed within and coupled to the frame, each leaflet comprising a main body having an outflow edge and an inflow edge, and two commissure tabs on opposite sides of the main body, wherein the leaflets are configured to move between an opened state allowing blood to flow through the frame from the inflow end to the outflow end and a closed state in which the outflow edges coapt with each other and block the flow of blood through the frame from the outflow end to the inflow end; wherein each commissure tab is paired with a commissure tab of an adjacent leaflet to form a plurality of commissures coupled to respective commissure support portions of the frame and having inflow ends and outflow ends, wherein the leaflets are tensioned across the outflow edges of the leaflets when the leaflets are in the opened state.

Example 270: The prosthetic heart valve of any example herein, particularly example 269, wherein the leaflets define an outflow channel that is tapered toward the outflow edges of the leaflets when the leaflets are in the opened state.

Example 271: The prosthetic heart valve of any example herein, particularly any one of examples 269-270, wherein when the leaflets are tensioned, a radial gap extends between the outflow edges of the leaflets and an inner surface of the frame.

In view of the many possible examples to which the principles of the present disclosure may be applied, it should be recognized that the illustrated examples are only preferred examples and should not be taken as limiting the scope of the present disclosure. Rather, the scope is defined by the following claims. We therefore claim all that comes within the scope and spirit of these claims.

We claim:

1. A prosthetic heart valve comprising:
a radially expandable and compressible frame comprising an outflow end portion, an inflow end portion having a plurality of inflow apices, and a plurality of cantilevered axial extensions, each axial extension having a fixed end and free end disposed between a pair of adjacent inflow apices; and
a plurality of valve leaflets disposed within and coupled to the frame, each leaflet comprising a main body having an outflow edge portion and an inflow edge portion coupled to a respective axial extension;
wherein the leaflet inflow edge portions are secured to the free ends of the axial extensions.

2. The prosthetic heart valve of claim 1, wherein the leaflet inflow edge portions and the free ends of the axial extensions are configured to move radially inwardly when the leaflets close under the back flow of blood and radially outwardly when the leaflets open under the forward flow of blood.

3. The prosthetic heart valve of claim 1, wherein the leaflet inflow edge portions and the free ends of the axial extensions are configured to move laterally toward adjacent inflow apices when a force is applied to the axial extensions.

4. The prosthetic heart valve of claim 1, the frame further comprising a central longitudinal axis extending from the inflow end portion to the outflow end portion, wherein the axial extensions are configured to resist radial movement toward the longitudinal axis of the frame when the leaflets close under the back flow of blood and away from the longitudinal axis when the leaflets open under the forward flow of blood.

5. The prosthetic heart valve of claim 1, the frame further comprising an outer skirt mounted to an outer surface of the frame, the outer skirt coupled to one or more axial extensions.

6. The prosthetic heart valve of claim 1, wherein the free ends of the axial extensions comprise an aperture.

7. The prosthetic heart valve of claim 1, wherein the free ends of the axial extensions are configured to compress in a circumferential direction of the frame when the frame is radially compressed.

8. The prosthetic heart valve of claim 1, the frame further comprising one or more commissure clasps, each clasp comprising a first commissure arm having a first indentation, a second commissure arm having a second indentation, and an opening therebetween configured to receive a commissure formed by two adjacent leaflets, wherein the first and second indentations are configured to receive a fastener such that the first and second commissure arms and the fastener restrict axial movement of the commissure received therein.

9. A prosthetic heart valve comprising:
a radially expandable frame comprising an inflow end, an outflow end, and a plurality of struts arranged to form a circumferentially extending row of struts forming the inflow end, wherein one or more selected struts have at least one opening extending therethrough; and
a plurality of leaflets disposed inside the frame and configured to regulate the flow of blood in one direction through the frame, each leaflet comprising an outflow edge portion and an inflow edge portion;
wherein the inflow edge portions of the leaflets are coupled to the selected struts of the frame with sutures extending through the openings.

10. The prosthetic heart valve of claim 9, wherein the selected struts are arranged in pairs of adjacent selected struts, wherein for a pair of adjacent selected struts, one of the selected struts is coupled to one of the leaflets and the other selected strut is coupled to an adjacent leaflet.

11. The prosthetic heart valve of claim 9, wherein each selected strut has one or more openings extending radially therethrough.

12. The prosthetic heart valve of claim 9, wherein each selected strut comprises an inflow section, a midsection, and an outflow section, wherein the openings radially extend through the midsection of the struts.

13. The prosthetic heart valve of claim 12, wherein the midsection of the selected struts has a circumferential width greater than a circumferential width of the inflow and outflow sections of the selected struts.

14. The prosthetic heart valve of claim 13, the frame further comprising a plurality of axially extending posts, wherein each of the posts comprises an indentation configured to receive the midsection of an adjacent selected strut when the frame is in a radially compressed state.

15. The prosthetic heart valve of claim 9, further comprising an outer skirt mounted to an outer surface of the frame and connected to the frame with sutures, wherein the sutures extend through the openings of the selected struts.

16. The prosthetic heart valve of claim 9, the frame further comprising a plurality of axially extending first posts and a plurality of axially extending second posts, wherein selected pairs of axially aligned first and second posts form a first set of selected posts and other selected pairs of axially aligned first and second posts form a second set of selected posts;
wherein the frame further comprises:
a first set of nuts coupled to the second posts of the first set of selected posts and a second set of nuts coupled to the second posts of the second set of selected posts, wherein the first set of nuts differ in at least one dimension from the second set of nuts; and
a plurality of first actuator members extending through the first set of selected posts and the first set of nuts, and a plurality of second actuator members extending through the second set of selected posts and the second set of nuts, wherein the first actuator members are configured to rotate in a first direction and the second actuator members are configured to rotate in a second direction, the first and second actuator members being configured to radially expand the frame from a radially compressed state to a radially expanded state.

17. The prosthetic heart valve of claim 9, the frame further comprising a plurality of axially extending posts, at least one post comprising an inner bore extending therethrough and an aperture extending from an external surface of the frame to the inner bore of the post.

18. A prosthetic heart valve comprising:
a radially expandable frame comprising an outflow end and an inflow end; and
a plurality of valve leaflets disposed within and coupled to the frame, each leaflet comprising a main body having an outflow edge and an inflow edge, and two commissure tabs on opposite sides of the main body, wherein the leaflets are configured to move between an opened state allowing blood to flow through the frame from the inflow end to the outflow end and a closed state in which the outflow edges coapt with each other and block the flow of blood through the frame from the outflow end to the inflow end;
wherein each commissure tab is paired with a commissure tab of an adjacent leaflet to form a plurality of commissures coupled to respective commissure support portions of the frame and having inflow ends and outflow ends, wherein the leaflets are tensioned across the outflow edges of the leaflets when the leaflets are in the opened state.

19. The prosthetic heart valve of 18, wherein the leaflets define an outflow channel that is tapered toward the outflow edges of the leaflets when the leaflets are in the opened state.

20. The prosthetic heart valve of 18, wherein when the leaflets are tensioned, a radial gap extends between the outflow edges of the leaflets and an inner surface of the frame.
